(19) United States
(12) Patent Application Publication
(10) Pub. No.: US 2026/0024623 A1
(43) Pub. Date: Jan. 22, 2026

(54) AUTOMATED DATA STORAGE SYSTEM

(71) Applicant: Atlas Data Storage, Inc., South San Francisco, CA (US)

(72) Inventors: Roger RUDOFF, South San Francisco, CA (US); Dominique TOPPANI, South San Francisco, CA (US); James DIGGANS, South San Francisco, CA (US); Mark WATSON, South San Francisco, CA (US); William BANYAI, South San Francisco, CA (US)

(21) Appl. No.: 19/339,450

(22) Filed: Sep. 25, 2025

Related U.S. Application Data
(63) Continuation of application No. PCT/US2024/021863, filed on Mar. 28, 2024.
(60) Provisional application No. 63/492,593, filed on Mar. 28, 2023.
(60) Provisional application No. 63/514,227, filed on Jul. 18, 2023.

(51) Int. Cl. G16B 50/30; B01J 19/00

(52) U.S. Cl. CPC G16B 50/30; B01J 19/0046; B01J 2219/00608

(57) ABSTRACT

Described herein are systems for data storage, including components for receiving, encoding, synthesizing, storing, reading, sequencing, and decoding digital information stored in polynucleotides and libraries of polynucleotides. Further described herein are assemblies for storing information, systems comprising the assemblies, and methods of using the assemblies.

### CROSS-REFERENCE TO RELATED APPLICATIONS

This application claims the benefits of priority to U.S. Provisional Patent Application No. 63/492,593, filed Mar. 28, 2023, and U.S. Provisional Patent Application No. 63/514,227, filed Jul. 18, 2023, the entireties of which are incorporated herein by reference. All publications, patents, and patent applications mentioned in this specification are herein incorporated by reference to the same extent as if each individual publication, patent, or patent application was specifically and individually indicated to be incorporated by reference.

### BACKGROUND

DNA is a compelling data storage medium given its superior density, stability, energy-efficiency, and longevity compared to current storage methods. However, automated systems may contain a number of different components execute steps such as encoding, writing/synthesizing, storing, and reading/sequencing. Therefore, there is a need to develop systems which can perform these functions.

### SUMMARY

Provided herein are systems for data storage comprising a computing system comprising at least one processor and instructions executable by the at least one processor to perform one or more operations, and a modular rack-mount synthesis. In some aspects, the one or more operations comprises: receiving digital information; encoding digital information in one or more nucleic acid sequences; and synthesizing a library of polynucleotides corresponding to the nucleic acid sequences. In some aspects, the modular rack-mount synthesis unit comprises: a computer controller, one or more reservoirs; at least one flow cell block comprising one or more flow cells; an extraction stage; a post-processing unit; a storage unit; a robotic interference; and/or a rack interface. In some aspects, the at least one flow cell block comprises at least 12 flow cells. In some aspects, the one or more flow cells comprise: one or more solid supports configured for synthesizing the library of polynucleotides; and one or more ports for exchanging gases, synthesis reagents, and/or extracted polynucleotides.

In some aspects, the modular rack-mount synthesis unit comprises the extraction stage and/or the post-processing unit, and the extraction stage and/or the post-processing unit comprises: an energy supply chain; and one or more ports for exchanging synthesis reagents and/or extracted polynucleotides. In some aspects, the modular rack-mount synthesis unit comprises the storage unit, and the storage unit comprises: a storage plate; a robotic interface configured to move storage plates; and an access port. In some aspects, the modular rack-mount synthesis unit comprises the rack interface, and the rack interface comprises: a power supply; a rack reagent bulkhead; a synthesis unit reagent bulkhead; one or more reservoirs; an energy supply chain; and a pilot valve bank. In some aspects, the storage unit provides the library of polynucleotides to a sequencing unit configured to sequence the library of polynucleotides.

In some aspects, the systems for data storage further comprise one or more sensors configured to: detect a status, wherein the status comprises a temperature, a pressure, a humidity, a change in voltage, a change in current, a capacitance, a conductivity, a storage plate position, a liquid volume, a flow rate, and/or a presence of a liquid; and report the status to the computing system. In some aspects, the modular rack-mount synthesis unit comprises the at least one flow cell block, and the one or more sensors are further configured to pause synthesis at the one or more flow cells or the at least one flow cell block. In some aspects, the system is a first system for data storage, and wherein the computing system is configured to divert encoded data from (i) a paused flow cell or (ii) a paused flow cell block to an active flow cell, an active flow cell block, or to a second system for data storage.

In some aspects, the computing system further comprises a cache, wherein digital information is retained in the cache until polynucleotides encoding the digital information are stored.

Further provided herein are assemblies for storing information comprising: a plurality of compartments comprising a first material, wherein the plurality of compartments is configured to receive a plurality of polynucleotides encoding information; a base plate comprising a second material; and a cover plate comprising a third material, wherein a coefficient of thermal expansion (CTE) of the first material, the second material, or both, is less than the third material. In some aspects, the plurality of compartments are positioned in an array on the base plate. In some aspects, the base plate, the cover plate, or both, comprise a plurality of recessed features, and wherein each compartment is located at least partially within a recessed feature. In some aspects, the first material comprises borosilicate. In some aspects, the second material has a specific heat capacity of about 0.5 J/k-° C. to 2.5 J/k-° C. In some aspects, the third material includes: a CTE of about 15 μm/m-° C. to 20 μm/m-° C.; a thermal conductivity of about 15 W/m-K to about 20 W/m-K; and/or a specific heat capacity of about 0.5 J/g-° C. In some aspects, the third material comprises stainless steel.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1A is a flowchart depicting a workflow for an exemplary automated data storage system using nucleic acids, including synthesis modules, according to aspects of the present disclosure.

FIG. 1B is a flowchart depicting a workflow for automated data storage system using nucleic acids, including storage modules. In some instances, these steps are performed by a robotics stage, according to aspects of the present disclosure.

FIG. 1C is a flowchart depicting a workflow of an exemplary system architecture at the level of a server rack (as described herein) for automated data storage system using nucleic acids including firmware, control systems (such as a network interface controller) and virtual data path, according to aspects of the present disclosure. In some aspects, data may move to the server rack for storage and validation, and the server rack may send data to an external interface for database management.

FIG. 2A is an illustration depicting an exemplary standardized DNA synthesis unit (DSU) with data/addressing, power, and reagent/DNA input/removal (a flow cell, flow cell block, or entire data storage unit), including a table describing dimensions and chip density for exemplary 6U module, according to aspects of the present disclosure.

FIG. 2B is a system level diagram depicting an exemplary automated data storage system using nucleic acids. Shown are modules for reagents, rack mount, synthesis, post-processing (e.g., deprotection, drying. PCR, purification), control, power distribution, and status indicators, according to aspects of the present disclosure.

FIG. 3A is an illustration depicting an exemplary rack unit for data storage, displaying a DNA data storage system unit extended from the rack unit, according to aspects of the present disclosure.

FIG. 3B is an illustration depicting a mechanical layout of the DNA data storage system unit shown in FIG. 3A, according to aspects of the present disclosure.

FIG. 3C is a diagram depicting a front/transparent view schematic of an exemplary DNA data storage system unit configured for use in a rack unit as shown in FIG. 3A, according to aspects of the present disclosure.

FIG. 3D is a diagram depicting a rear panel schematic for an exemplary DNA data storage system unit, according to aspects of the present disclosure.

FIG. 4A is a two-diagram figure depicting a mechanical layout of the extraction stage of an exemplary DNA data storage system unit, according to aspects of the present disclosure. The top diagram illustrates an extraction stage wherein the storage chamber is present in a first location for dispensing at the front of the stage. The bottom diagram illustrates an extraction stage wherein the storage chamber is moved to a second location at the rear of the stage for waste/washing. These two locations are shown for example purposes only; in some instances the chamber moves to any location associated with a flow cell block.

FIG. 4B is a diagram depicting a side-view fluidic path of an exemplary DNA data storage system unit, including reagent input, according to aspects of the present disclosure.

FIG. 4C is a diagram depicting a top-view fluidic path of an exemplary DNA data storage system unit, including reagent input, according to aspects of the present disclosure.

FIG. 4D is a diagram depicting a fluidic path schematic for an exemplary flow cell block, including control valves.

FIG. 4E is an illustration depicting an exemplary flow cell block device showing fluid inputs. 12 flow cell units are shown as an example only.

FIG. 5A is a three-diagram figure depicting schematics of an exemplary flow cell block for synthesis including fill, wash, and dry steps using Burkert valves (6712 series, 24V), according to aspects of the present disclosure.

FIG. 5B is a three-diagram figure depicting schematics of an exemplary flow cell block for extraction including clean, fill, and extract steps using Burkert valves (6712 series, 24V), according to aspects of the present disclosure.

FIG. 5C is a diagram depicting a schematic of an exemplary flow cell block for extraction with angles alpha (α) and beta (β) labeled, and x-axis, y-axis, and z-axis labeled, according to aspects of the present disclosure.

FIG. 6 is a piping and instrumentation diagram depicting an exemplary 12 flow cell unit design, according to aspects of the present disclosure. Each flow cell is depicted with an inlet and an outlet. Inlets supply reagents for biomolecule synthesis. Outlets provide a fluidic path for waste products and can also be put under positive pressure from gas sources (e.g., nitrogen). Inlets can also be bypassed for biomolecule extraction.

FIG. 7 is a diagram depicting a process schematic for liquid phase deprotection of nucleic acids in a device described herein, according to aspects of the present disclosure. Steps include one or more of loading the chip into the flow cell (FC), filling the flow cell with deprotection solution, closing the valve and incubating for a period of time, opening the valve to collect liquids comprising polynucleotides, incubating in AMA (ammonium hydroxide/40% aqueous methylamine 1:1 v/v) at 65° C. for 20 minutes or incubating with TBA (tert-butylamine), and drying down the tubes.

FIG. 8 is a diagram depicting a schematic for a nucleic acid data storage system with modules for input, synthesis, storage, amplification/processing, and sequencing manipulated by a controller and robotic system, according to aspects of the present disclosure. In some aspects, systems described herein comprise one or more of these types of units.

FIGS. 9A-9I are illustrations depicting exemplary structures for storing a plurality of polynucleotides, according to aspects of the present disclosure. Structures used for nucleic acid storage may be stored in on a plate or other device described herein. FIG. 9A depicts a structure that is substantially tubular. FIG. 9B depicts a structure comprising a cap and a body that are flush-welded together. FIG. 9C depicts a structure comprising a removable screw-cap. FIG. 9D depicts a structure comprising a septum. FIG. 9E depicts a structure comprising two rounded, pill-shaped halves that form a seal when one half is inserted into the other. FIG. 9F depicts a structure comprising a substantially flat, disc-shaped chamber with sealable lid. FIG. 9G depicts a structure comprising a box, optionally with an attached lid. FIG. 9H depicts a structure comprising a superficial radio frequency identification (RFID) tag. FIG. 9I depicts a structure comprising an embedded RFID tag.

FIG. 10 is a diagram depicting an exemplary computing device with one or more processors, memory, storage, and a network interface, according to aspects of the present disclosure.

FIG. 11 is a flowchart depicting an exemplary method for storing digital information in a plurality of polynucleotides, according to aspects of the present disclosure.

FIG. 12 is a flowchart depicting an exemplary method for retrieving digital information in a plurality of polynucleotides, according to aspects of the present disclosure.

FIG. 13 is a diagram depicting an exemplary passive RFID system, according to aspects of the present disclosure.

FIG. 14 is a diagram depicting a non-limiting example of digital information divided into a plurality of sub-items for storage in structures, according to aspects of the present disclosure.

FIGS. 15A-15B are illustrations depicting side views of an exemplary compartment for storing polynucleotides, according to aspects of the present disclosure. FIG. 15A depicts a first set of dimensions of the compartment with a pipette illustrating filling or retrieving the polynucleotides in solution. FIG. 15B shows the same side view as FIG. 15A, with further dimensions that may not be shown in FIG. 15A.

FIG. 16 is an illustration of an exemplary cover plate or a base plate of a system for storing polynucleotides, according to aspects of the present disclosure.

FIG. 17 is an illustration of an exemplary cover plate before (solid lines) and after (dotted lines) exposure to heat, according to aspects of the present disclosure. In some aspects, the expansion of the cover plate is exaggerated for the purposes of illustration.

FIG. 18 a schematic diagram depicting a cross-section of an exemplary assembly for storing polynucleotides, according to aspects of the present disclosure. The assembly comprises a base plate, compartments for holding the polynucleotides, and a cover plate. In some aspects, the wall angle of the cover plate wall and/or the base plate wall is exaggerated for purposes of illustration, and the wall angle of the cover plate wall and/or the base plate wall may be less than 0.5°.

FIG. 19 is a table reporting properties of various types of glass (GA-1, GA-4, GA-9. GA-12, GA-13, GA-21, GA-34, GA-44, and GA-47) that may be selected for a compartment for storing polynucleotides, according to aspects of the present disclosure.

FIG. 20 is a flowchart depicting an exemplary method for scaling the plurality of polynucleotides in a storage device, according to aspects of the present disclosure.

### DETAILED DESCRIPTION

### Data Storage Systems

Currently available data storage technologies—based on hard disk or tape drives—are facing significant physical, technological and investment challenges. This has greatly slowed the rate of growth in storage capacity which, together with the need to migrate data every 5-10 years (depending on the storage medium), significantly impacts the costs of long term (>10 years) storage. In addition, the proportion of long term (“cold”) data, relative to active (“warm”/“hot”) data is increasing dramatically which, when combined with the slow-down in data density growth, puts pressure on data center infrastructure utilization, footprint and storage capacity. In contrast, systems, devices, and methods provided herein, which utilize DNA molecules as the storage medium, offer increases in both data longevity/retention times and volumetric storage density.

Additionally, to ensure the stability and viability of DNA used to store data for extended periods (e.g., greater than 30 years) it can be important that the encapsulation method protects the encapsulated DNA from external contamination. This can be especially true for ingress of water, but also includes other deleterious gases and liquids. Current method may generally be directed to long-term DNA storage on the scale of thousands of years, for example, encapsulating desiccated DNA in a borosilicate glass insert sealed in small metallic shells and laser welding them shut in an inert atmosphere. Accelerated aging experiments have indicated that the DNA half-life, when stored in ambient conditions, may be as high as 38,000 years.

However, while currently available methods and systems may be used in archival long term DNA data storage due to the projected longevity of stored DNA, they have numerous disadvantages for use in long term enterprise or hyperscale archive storage applications, for example, for data storage applications including data center/rack scale applications. Specifically, not only can the materials themselves expensive, but the sealing techniques can involve a high power (YAG) laser to laser weld the tops of the capsules, and an associated robotic automation system to place and locate individual capsules to facilitate the welding process. These processes can be cumbersome, expensive, and unreliable, can require high maintenance, can have slow throughputs, and/or can take up a very large amount of space.

Further, the capsules storing the DNA themselves can be relatively large, and importantly, are considerably larger than the mass of the DNA it contains, thereby significantly reducing the volumetric storage capacity achievable. These constraints make it such that these systems cannot compete with the volumetric storage capacity of conventional data tapes (e.g., LTO 10 cartridges). The systems and methods described herein may overcome such limitations, while retaining the long-term storage advantages.

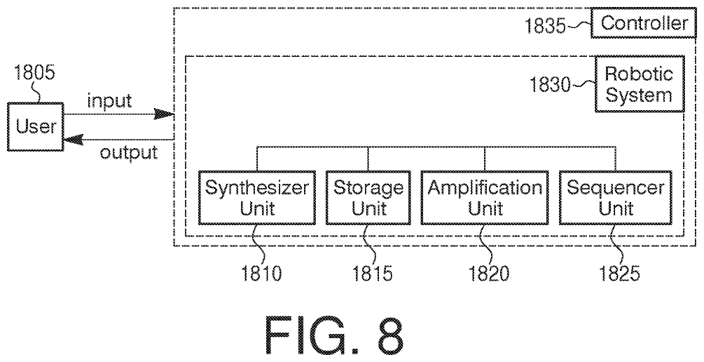

Provided herein are data storage systems that utilizes highly modular synthesis, processing and storage framework. In some instances, such systems are based on a standard size. In some instances, such systems are based on an OCP (open compute) data center rack form factor which simplifies data center utilization and adoption. In some instances, such systems are based on a standard OCP (21″/48OU) data center rack form factor which simplifies data center utilization and adoption. In some instances, such systems are based on a standard (19″/48U) data center rack form factor which simplifies data center utilization and adoption. Further provided herein are systems incorporating many tens to hundreds of individual synthesis/flow cells which are integrated into a single synthesis module, which is serviced remotely. In some instances, to access the DNA synthesized by each individual flow cell, a central robotic system is used to position a storage plate, containing many tens to hundreds of storage chamber (or containers. e.g., wells or capsules) to be filled automatically. In some instances, the robotic system then moves the filled plate to additional modules, located within the storage rack, for subsequent post processing steps including but not limited to intermediate dry-down, PCR, aliquoting for quality control and monitoring, final dry down for storage and plate sealing. In some instances, a robotic system moves the processed and sealed plate to a storage module for long term (e.g., up to decades) storage. In some instances, systems provided herein interface with magnetic or solid-state memory systems. In some instances, a data storage system comprises one or more components shown in FIG. 8. Provided herein are systems for data storage comprising one or more of: a computing system comprising at least one processor and instructions executable by the at least one processor to perform operations comprising: receiving digital information; encoding digital information in one or more nucleic acid sequences; and synthesizing a library of polynucleotides corresponding to the nucleic acid sequences; and one or more modular rack-mount synthesis units. In some instances, a data storage system comprises one or more of a computer controller, a flow cell block; an extraction stage; a post-processing unit; a storage unit; a robotic interface; and a rack interface.

Further provided herein are systems and methods for encapsulation. In some instances, the systems and methods described herein utilize a hermetic sealing method, and in some examples, specifically applied to DNA data storage. In some instances, the systems and methods described herein reduce the complexity of the sealing mechanism compared to those currently available. In some instances, the volume of the encapsulation is also reduced, for example, in some embodiments, by using small compartments, such as vials, enclosed in a thin metallic cover plate. Further, in some instances, adding a metallic base plate made of the same or a similar material as the cover plate provides high mechanical rigidity and/or a very thin profile/form factor for the final assembly. In some instances, the method comprises hermetic sealing under an inert atmosphere. In some instances, the hermetic sealing method comprises a glass-to-metal hermetic sealing method. Glass-to-metal sealing may generally be used to create robust feedthroughs into a vacuum-tight package. Glass-to-metal sealing may generally be accomplished with two different mechanisms; compression sealing or matched sealing. Compression seals can employ a coefficient of thermal expansion (CTE) mismatch between a glass insert and an outer housing. In some instances, hermetic seal can be established because the thermal compression of the outer housing acts on the glass insert as the parts cool down after heat treatment. Housing materials with a high CTE, such as stainless steels or titanium, may be suited for compression seals. Depending on design, a compression seal can withstand a very high level of pressure (e.g., up to 3000 bars) and this type of glass-to-metal seal can be, among other uses, suited for ultra-high vacuum windows, components utilized in space vehicles, high-pressure sensors for use in industrial and oil and gas application and in microelectronics connectors, including defense applications.

Provided herein are devices or assemblies for storing information. In some instances, an assembly for storing information can comprise one or more of: a plurality of compartments, a base plate, and at least one cover plate. In some instances, the plurality of compartments comprise a first material. In some instances, the base plate comprises a second material. In some instances, the at least one cover plate comprises a third material. In some instances, provided herein are methods for storing information. In some instances, a method for storing information comprises one or more of: providing an assembly comprising a plurality of compartments, a base plate, and a cover plate; and generating a temperature gradient between the base plate and the cover plate. In some instances, the plurality of compartments comprise a plurality of polynucleotides. In some instances, the temperature gradient causes the base plate, the cover plate, or both to expand or contract to seal the plurality of compartments. In some instances, provided herein are systems for storing information. In some instances, a system for storing information comprises one or more of: an assembly for storing information; a material deposition system; one or more temperature control systems; and a computing system. In some instances, the assembly comprises one or more of: a plurality of compartments; a base plate; and at least one cover plate. In some instances, a material deposition system comprises a dispenser. In some instances, the dispenser deposits a plurality of polynucleotides to a compartment of the plurality of compartments. In some instances, the one or more temperature control systems is used for heating or cooling the base plate, the cover plate, or both. In some instances, a computing system comprising at least one processor and instructions executable by the at least one processor to perform one or more operations. In some instances, the one or more operations comprises orchestrating movement of one or more components of the system; monitoring a property of the one or more components of the system, or both.

Further provided herein are methods and systems for tracking content in a DNA data storage system. Digital information can be encoded in a plurality of polynucleotides stored in structures of a DNA data storage system. The structures can include a tag for identifying the structure, providing metadata relating to the content in the structure, or a combination thereof. The tag can be used as a label, a file system, or a combination thereof. For example, the tag can be used to remotely catalogue the structures within the data storage system, and allow individual identification and selection of the structure from the data storage system. The tag can also provide fixity of the data as the tag can provide direct physically associated information regarding the content of the structure.

In some instances, the systems store digital information. In some instances, the system comprises a plurality of polynucleotides collectively encoding digital information. In some instances, the systems further comprise a structure for storing the plurality of polynucleotides. In some instances, the structure comprises a radio frequency identification (RFID) tag. In some instances, the RFID tag comprises metadata relating to the plurality of polynucleotides. In some instances, the methods provide for storing digital information. In some instances, the methods comprise synthesizing a plurality of polynucleotides. In some instances, the plurality of polynucleotides collectively encode digital information. In some instances, the methods comprise writing metadata relating to the plurality of polynucleotides in a radio frequency identification (RFID) tag. In some instances, the methods comprise storing the plurality of polynucleotides in a structure. In some instances, the structure comprises the RFID tag. In some instances, the methods determine data integrity of stored digital information. In some instances, the methods comprise writing metadata relating to the plurality of polynucleotides to a radio frequency identification (RFID) tag. In some instances, the methods comprise scanning the RFID tag after a duration of time to determine data integrity. In some instances, the RFID tag is valid or invalid. In some instances, provided herein are articles of manufacture. In some instances, an article of manufacture comprises a structure for storing a plurality of polynucleotides encoding digital information. In some instances, the structure comprises a radio frequency identification (RFID) tag. In some instances, the RFID tag comprises metadata relating to the plurality of polynucleotides.

### Data Storage Unit

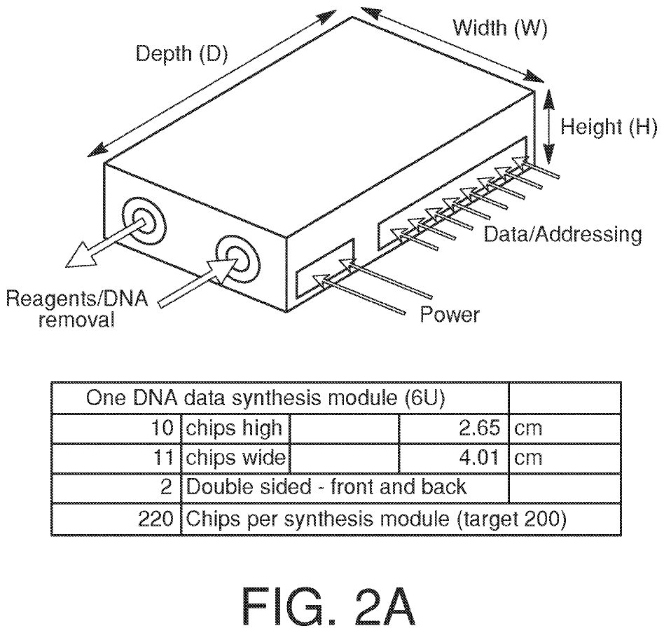

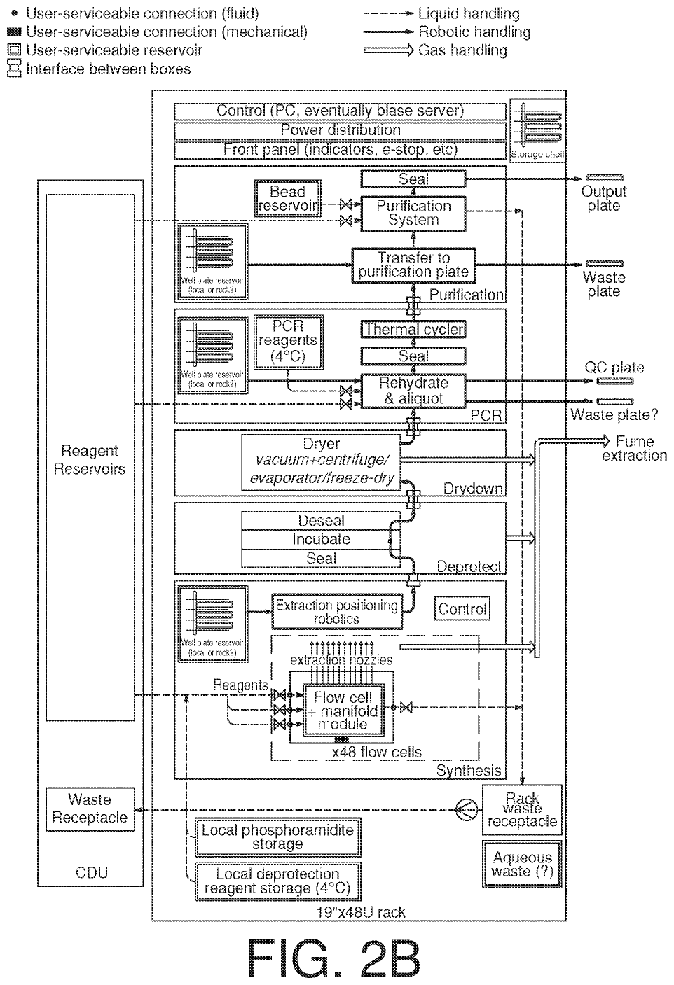

Provided herein are data storage systems. In some instances, a data storage system comprises the system of FIG. 2A. In some instances, systems comprise one or more ports for reagent/gas/waste exchange, interfaces for data processing/communication, power, and a plurality of solid supports for synthesis of biomolecules. A data storage system in some instances comprises one or more modules of the system shown in FIG. 2B. An exemplary system provided herein in some instances comprises one or more of a reagent reservoir, well plate reservoir, waste receptacle (or reservoir), and a rack unit, and one or more modules. In some instances modules include but are not limited to synthesis, deprotection, storage, post-processing (after synthesis or after cleavage from a solid support, e.g., deprotection, dry down, amplification, purification, or other process), control module, power distribution module, and status indicator (e.g., a panel). In some instances, a rack interface comprises one or more (local) reservoirs. In some instances, reservoirs are configured to hold waste (organic or aqueous), phosphoramidites, amplification reagents (e.g., PCR or other amplification technique), and beads.

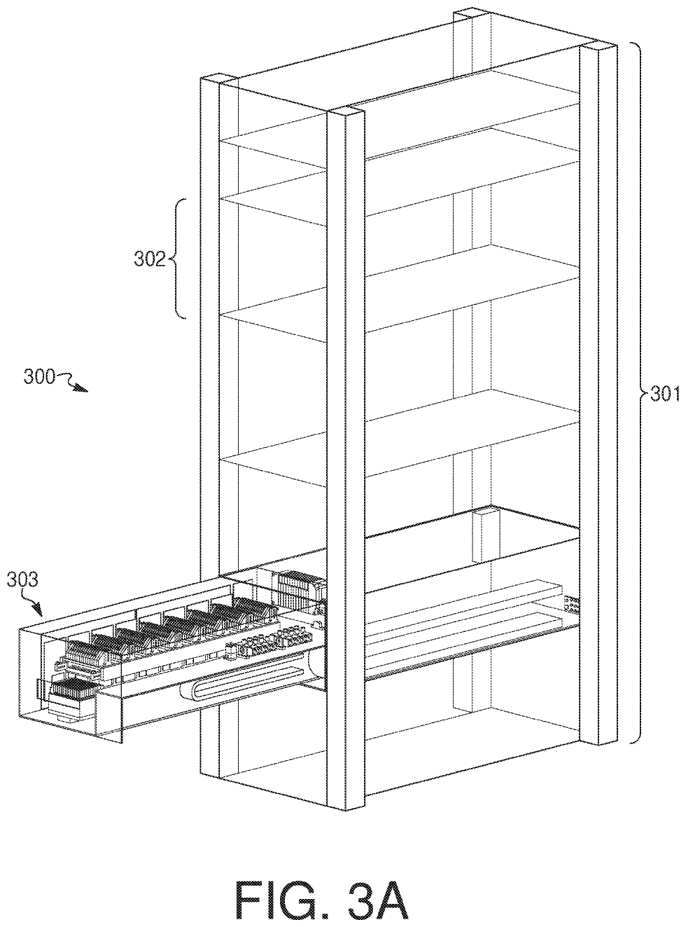

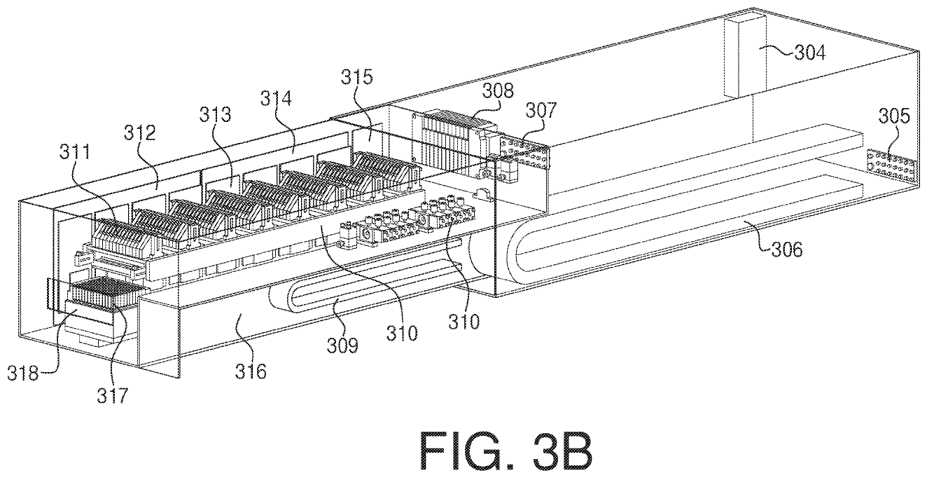

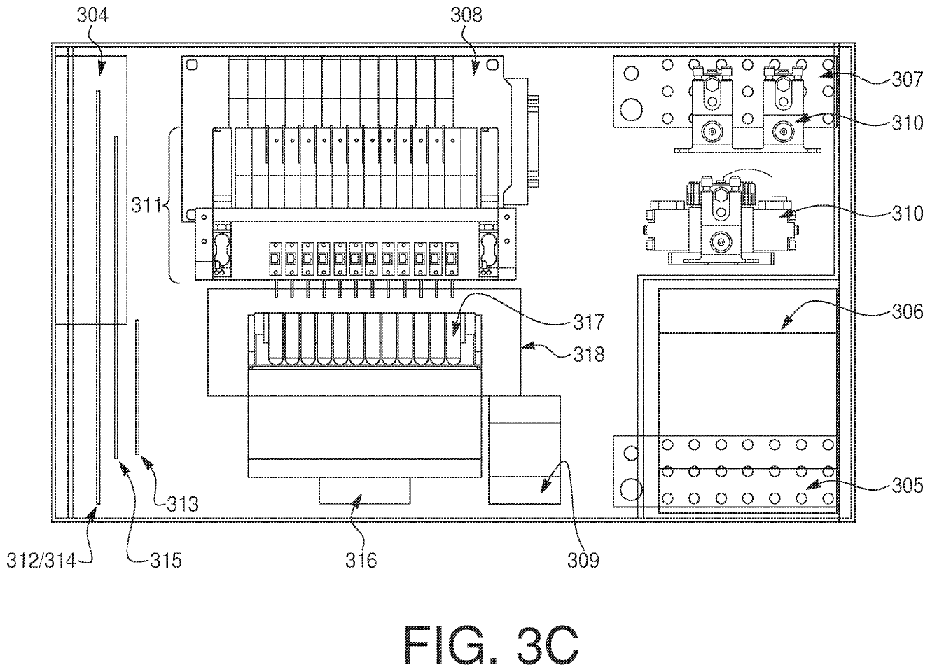

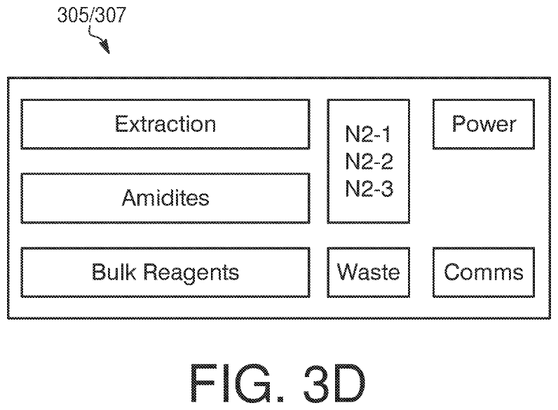

A data storage may comprise the system of 300 (FIG. 3A). In some instances, biomolecules comprise nucleic acids (e.g., DNA. RNA), peptides, sugars, or other biopolymer. In some instances, data storage systems comprise a data storage unit 303. In some instances, data storage units are configured to mount into a rack unit 301 (FIGS. 3A-3C). In some instances a rack unit comprises a plurality of “slots” 302 where a data storage unit is inserted. In some instances, a data storage unit fills one or more slots in a rack unit. Rack units 301 in some instances are used as part of large data storage centers comprising hundreds or even thousands of rack units in order to store large amounts of data. In some instances, a plurality of data storage units enables data redundancy. Rack units in some instances comprise a plurality of data storage units, such as 1, 2, 3, 4, 5, 6, 7, 8, 9, 10, or more than 10 data storage units. In some instances, data storage units provided herein are highly modular, allowing facile exchange of an entire data storage unit, or components such as flow cell blocks, flow cells, storage media, or solid supports for biomolecule synthesis. In some instances a rack-mount data storage unit provided herein is compatible with a standard (19″/48U) rack unit. In some instances a rack-mount data storage unit provided herein is compatible with a standard OCP (21″/48OU) rack unit.

### Rack Interface

Data storage units may be configured for interface with a rack unit. In some instances, data storage units are configured to be movable within the rack unit (example of an “open” data storage unit is shown in FIGS. 3A-3C. In some instances, chains, guides, slides, tracks, rollers, cables, magnets, or other mechanical method is used to enable the data storage system to slide laterally away from the rack unit (e.g., “open”) and slide laterally back into the rack unit (e.g., “closed”). In some instances, this enables user access to one or more components of the data storage system (e.g., manually user access or robotic system access). In some instances, this enables complete removal of the data storage unit from the rack. In some instances, removal of a data storage unit from a rack unit does not require use of additional tools or removal of rivets, screws, or bolts. In some instances, a user or robotic system removes a faulty storage device from the rack and replaces it with a functional storage device. In some instances, a rack unit comprises a fan, filter, or other means for extracting fumes generated in the system.

A rack interface in some instances provides power, communications, input of gases, reagents and removal of waste, access to certain components of the data storage unit, and performs other functions needed for data storage. In some instances, the interface comprises one or more connectors (or ports). In some instances, connectors or ports are configured for modular removal of the entire synthesis unit or various components of the synthesis unit from the rack. In some instances, the rack interface delivers power, delivers communications, inputs gases and reagents, and removes waste from a main reservoir outside the rack unit (e.g., reagent bulkhead 305/307). In some instances, an interface comprises one or more e-chains (e.g., 306/309) which are configured to deliver power, reagents, gases, or remove waste to various components of the data storage unit. In some instances, a rack interface comprises one or more panels. Panels are in some instances located on any part of the system, including a top, bottom, sides, front or rear. In some instances, a rack interface comprises a front panel and a rear panel. In some instances, a rear panel interface comprises one or more connectors and ports such as those shown in FIG. 3D (305/307). In some instances one or more panels are connected via wiring or tubing (e.g., two bulkheads, such as 305 and 307). In some instances, a panel comprises an access window 318. In some instances, an access window is configured to remove one or more storage plates or chambers from a storage unit. In some instances, a panel comprises one or more connectors for running diagnostics on a storage unit. In some instances, a front panel comprises one or more gauges, dials, indicator lights, LCD displays, or other indicator of a data storage unit's status or performance. In some instances, a panel indicates the status of one or more sensors. The one or more sensors can comprise, by way of non-limiting example, liquid volume sensor, capacitance, a temperature sensor, humidity sensor, a pressure sensor, a salinity sensor, a light sensor, a UV sensor, conductivity, or a gas sensor (e.g., an O₂ or N₂ sensor). In some instances, a sensor is integrated into one or more flow cells. In some instances a sensor is configured to identify a reagent or liquid in a flow cell. In some instances, a sensor comprises an out of plane cathode in the flow cell. In some instances, a panel indicates the presence of fault states such as one or more of loss of power, low gas pressures, and leaking fluid. In some instances, a panel comprises a control panel for a module or unit of a system described herein. In some instances, a panel comprises an interface comprising a visual indicator of status and/or fault indication. In some instances, the fault states are associated with a fault detected by the one or more sensors. In some instances a panel comprises connectors for one or more of extraction, amidites, bulk reagents, waste, power, communications, and gases. In some instances, gases comprise nitrogen or argon. In some instances, at least 2, 3, 4, or at least 5 different gas pressures are delivered through one or more connectors on a panel. In some instance, a rack interface comprises one or more bulkheads. In some instances, a communications interface comprises a wireless communications interface.

In some instances, an interface comprises a connector for power. In some instances, the power connector delivers approximately 110 or 220 volts to the rack unit. In some instances, the rack unit further comprises a transformer 304 (or power supply) to deliver 24V to the synthesis unit. In some instances, a rack interface comprises one or more wiring harnesses. In some instances, wiring harnesses are configured as an e-chain. In some instances, wiring harnesses provide communications and/or power to various components of the data storage unit, such as circuit boards, valves, sensors, robotics, pumps, or other component. In some instances, an interface further comprises a battery backup system. In some instances, the backup system is enabled when the entire rack or one or more data storage units in the rack loses power. In some instances, loss of power is reported to a central controller, processor, display panel, or remote workstation. In some instances, communication comprises information for operation of the data storage unit. In some instances, communication comprises digital information to be encoded in biomolecules.

A rack interface may comprise one or more e-chains for distribution of electricity, liquids, and/or gases. In some instances, an e-chain is configured for delivery of reagents and/or removal of waste 306. In some instances, an e-chain is configured for extraction of biomolecules from a synthesis unit or storage stage 309. In some instances, an e-chain is attached to rear panel ports or connectors. In some instances, an e-chain is connected to one or more reservoirs. In some instances, a reservoir comprises a reagent reservoir. In some instances, a reservoir comprises an extracted biomolecule reservoir. In some instances, a reservoir comprises a waste reservoir. In some instances, the e-chain comprises an extraction stage e-chain 309. In some instances, an e-chain is shielded or protected from electrical shorts, interference, or contact with solvents used during biomolecule synthesis. In some instances, an e-chain allows extension of the data storage unit laterally or away from the rack unit without disconnecting electricity, reagents, waste, or gas. In some instances, fluids and/or gases are routed throughout a data storage unit using tubing. In some instances, tubing comprises about 1/64″, 1/32″, 1/16″, or about ⅛″ ID tubing.

A rack interface may comprise a valve bank 308. In some instances, the valve bank comprises a pilot valve bank. In some instances, a valve bank comprises at least 4, 8, 12, 16, 24, 36, 48, or at least 64 channels. In some instances, a valve bank comprises no more than 4, 8, 12, 16, 24, 36, 48, or no more than 64 channels. In some instances, a valve bank comprises 4-64, 4-48, 4-32, 8-64, 8-48, 8-48, 8-32, 8-24, 12-36, 12-48, 12-64, 16-36, 16-64, 16-48, 20-30, 24-36, 24-48, 24-64, or 48-64 channels.

A rack interface may comprise an open compute (OCP) rack design. In some instances, a system comprises an OCP specification V1.2 or V2.0. In some instances, an open compute design comprises a 21″ standard with 600 mm width. In some instances, OCP comprises a 12V/48V DC electrical power supply. In some instances, OCP comprises one or more DC busbars. In some instances, busbars are rear-mounted DC busbars. In some instances, OCP comprises up to three 12 V DC busbars, each of which can additionally be divided horizontally into 2 power zones. In some instances, outputs of 6.6 kW and 13.2 kW all the way up to 40 kW are provided. In some instances OCP comprises a 48V DC electrical power supply, providing up to 36 kW of electrical output is available for each rack. In some instances, OCP server hardware is supplied via an end-to-end DC busbar for each rack. In some instances, a rack comprises an OCP power packs. In some instances, a power pack is configured to generate the direct current required from a 3-phase alternating current source. In some instances, power packs are installed in the racks to power the data storage system. These power packs in some instances support modular rack mounting and monitoring via the network (via SNMP). In some instances, individual power pack blocks generate typical outputs of 3 kW and are connected in parallel. In some instances, an OCP comprises a battery backup units integrated into the OCP rack to supply backup power.

### System Operations

Provided herein are data storage systems configured to perform one or more methods (or operations) described herein. In some instances, data storage systems comprise one or more modules or components configured to perform methods described herein. In some instances, a system comprises one or more modules provided in FIG. 8.

A computer interface may be used to control input for a data storage system provided herein. In some instances, a computer interface is configured to receive data (e.g., from a user, customer, server, computer, another data storage system, or other source). In some instances, a computer interface is controlled by a user. In some instances, a computer interface is fully automated without substantial human operation. In some instances, a computer interface comprises formatting or converting input data. In some instances, a computer interface comprises encoding, encrypting, decoding, or decrypting data. In some instances a computer interface provides an output comprising instructions (e.g., formatted data) for synthesis of biomolecules (e.g., nucleic acids). In some instances, a computer interface provides synthesis instructions for one or more synthesis units.

A data storage system provided herein in some instances comprises an interface for receiving reagent information. In some instances, reagent information comprises reagent age, storage conditions, amount (e.g., volume), or other data. In some instances the interface is configured to receive information from one or more sensors located at or near reservoirs. In some instances one or more reservoirs are configured to store reagents or waste. In some instances, an interface receives information on reagent quality for recycled/recovered reagents. In some instances, the interface provides an output to a server, cloud, user, or display panel on the system.

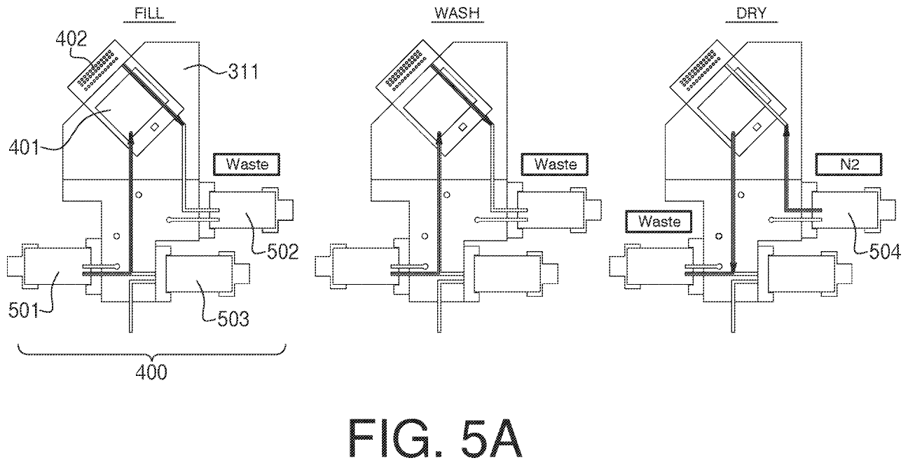

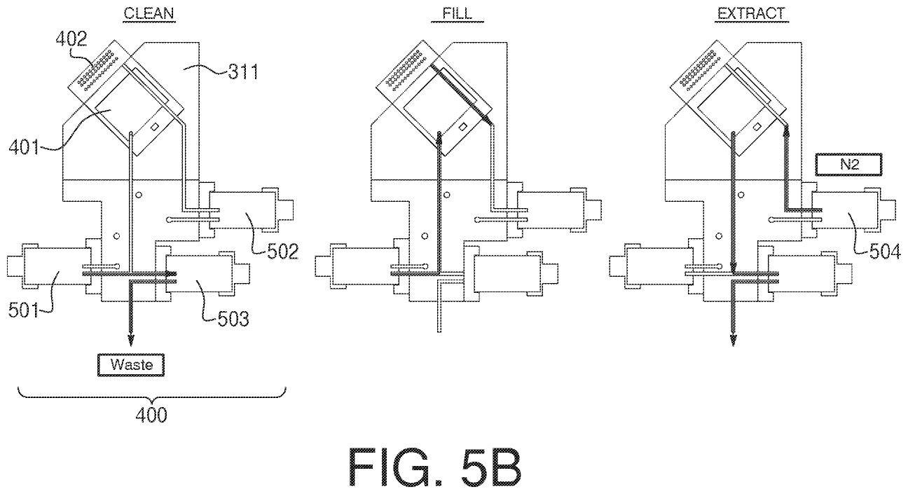

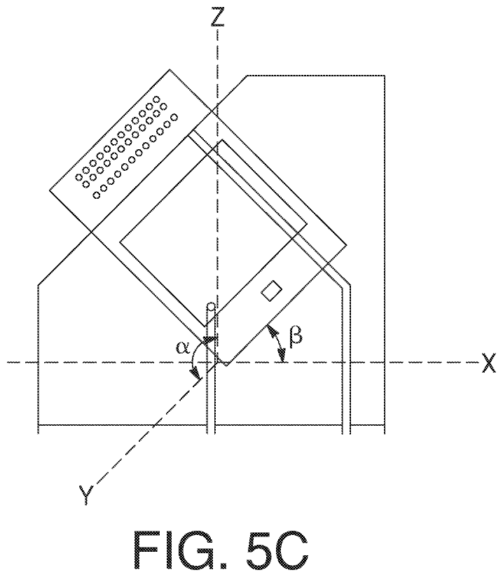

A synthesis unit may convert formatted data into one or more biomolecules. In some instances, formatted data comprises an in silico biopolymer sequence. In some instances the biopolymer sequence comprises a nucleic acid sequence. In some instances, the synthesis unit is configured to receive instructions for synthesis through a communication port. In some instances, instructions comprise programs for controlling valves, temperature, humidity, pumps, monitoring sensors, apply power to one or more components of the synthesis unit, or other instructions related to the synthesis of biomolecules. In some instances, a synthesis provides as an output quality control information relating to the synthesis process. In some instances, a synthesis provides as an output storage information regarding the synthesized molecules such as memory structure, flow cell identification information, flow cell block identification, total operation time, or other information related to biomolecules synthesized in a synthesis unit. In some instances, a synthesis unit receives instructions to cleave or remove biomolecules from one or more solid supports. In some instances, a synthesis unit receives instructions to transfer cleaved biomolecules to an extraction unit. In some instances, all biomolecules are cleaved from one or more solid supports. In some instances, a percentage of biomolecules are cleaved from one or more solid supports. In some instances, at least 5%, 10%, 15%, 20%, 25%, 50%, 75%, 90%, 95%, or at least 99% of biomolecules cleaved. Exemplary operations for synthesis is shown in FIG. 5A. In some instances, operations control fluid communication between solvents, reagents, and gases and the synthesis surface on a solid support 402. In a fill step, a flow cell 401 inside a flow cell block 311 is filled with a reagent by opening a first valve 501. Any liquid or gas present in the flow cell from a previous step is moved to liquid waste (e.g., reservoir) by opening a second valve 502 (FIG. 5A, left). A third valve 503 remains closed. In a wash step, similar valves are opened and closed but instead a wash solvent flows through the system (FIG. 5A, middle). In a dry step, a fourth valve 504 connected to a gas is used to push out any fluid from the flow cell 401 into liquid waste via a first valve 501 (FIG. 5A, right). The third valve 503 remains closed. In a clean step, liquid is pumped from a first valve 501 to a waste via a third valve 503 (FIG. 5B, left). A second valve 502 remains closed. During an extract step, a fourth valve 504 connected to a gas is used to push out any fluid from the flow cell 401 into an extraction chamber via a third valve 503 (FIG. 5A, right). A first valve 501 remains closed.

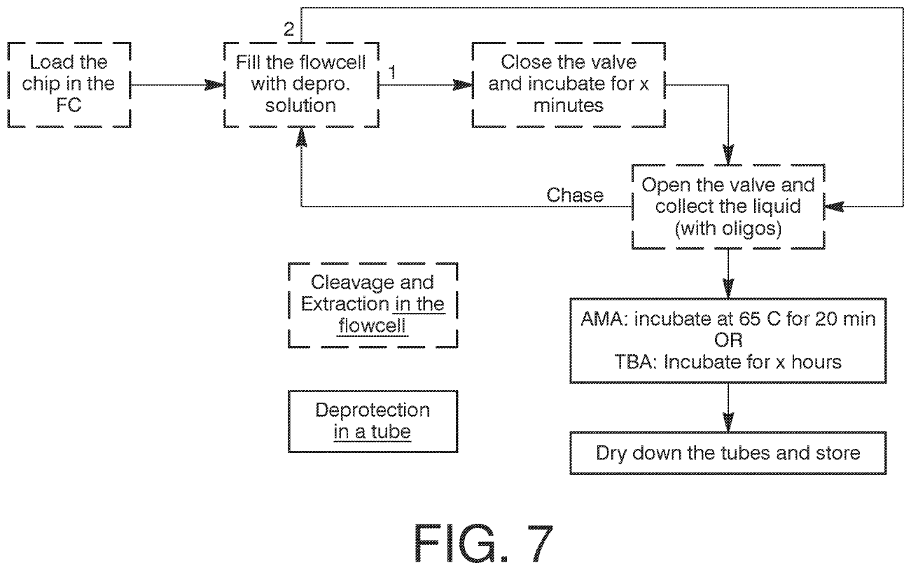

An extraction unit 316 may receive synthesized biomolecules from a synthesis unit. In some instances, an extraction unit is configured to perform one or more post processing steps after synthesis. Post processing steps may include but are not limited to concentrating, drying, amplification, purification, quality control/analysis, cleavage, ligation, selection/capture, deprotection, or other post-processing step. In some instances, an extraction unit receives instructions to perform a drying step on biomolecules after synthesis. In some instances, an extraction unit receives instructions to perform an amplification step on biomolecules after synthesis. In some instances, an extraction unit receives instructions to store biomolecules in a storage unit. In some instances, an extraction unit receives instructions to test synthesized molecules for quality or quantity. In some instances, an extraction unit is configured to perform one or more steps of FIG. 7. In some instances, an extraction unit is configured to do one or more of: fill a flow cell comprising a solid support for synthesis with a deprotection solution; close one or more valves and incubate the flow cell; open one or more valves and collect extracted biomolecules; and optionally deprotect biomolecules after extraction.

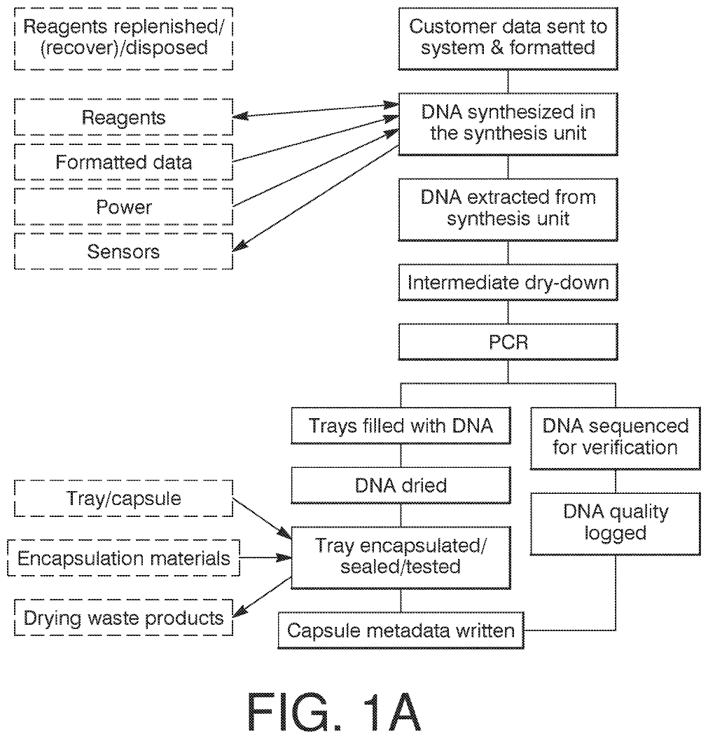

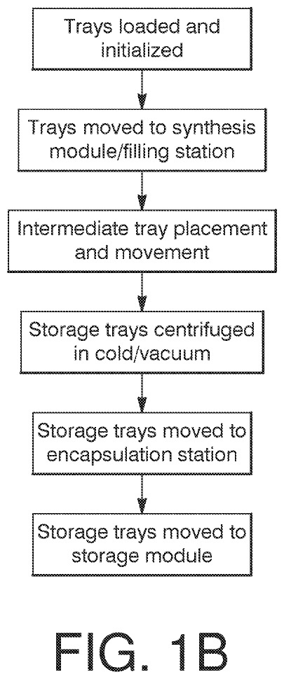

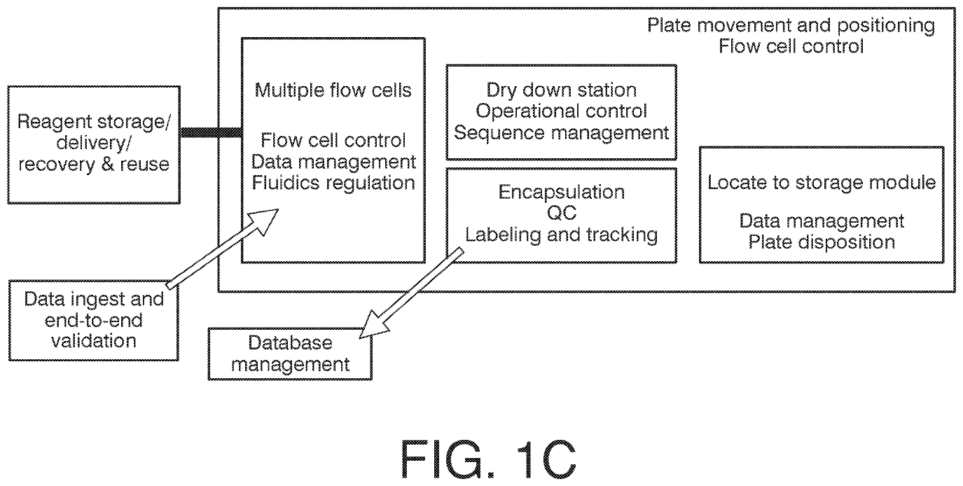

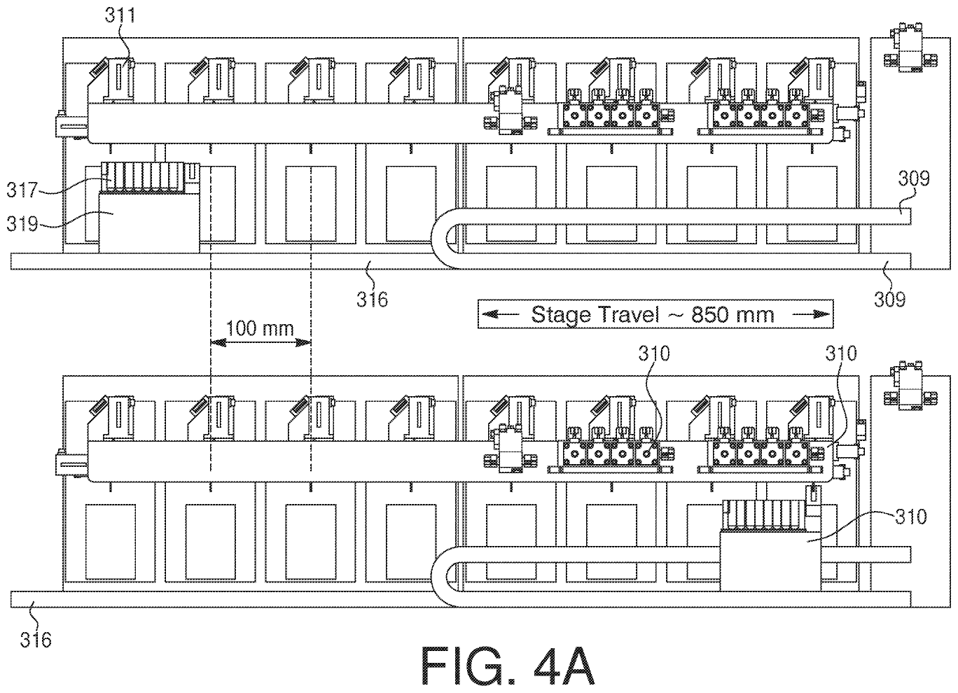

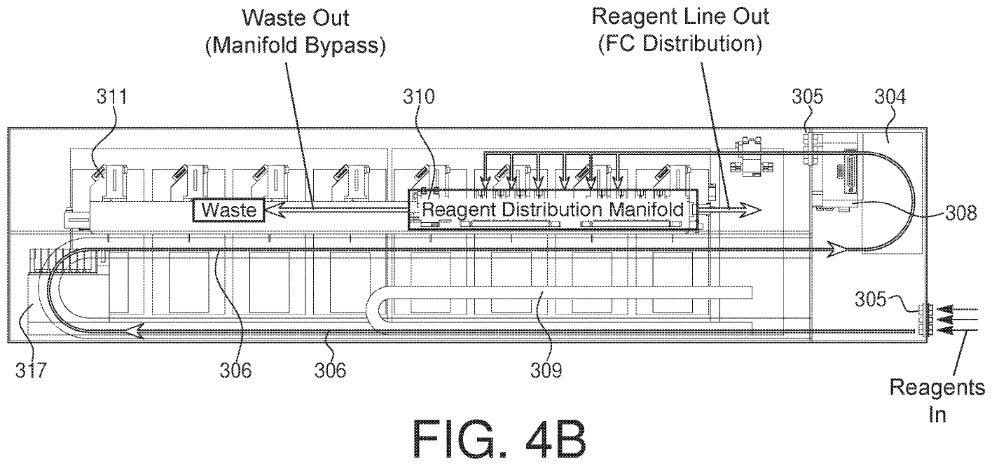

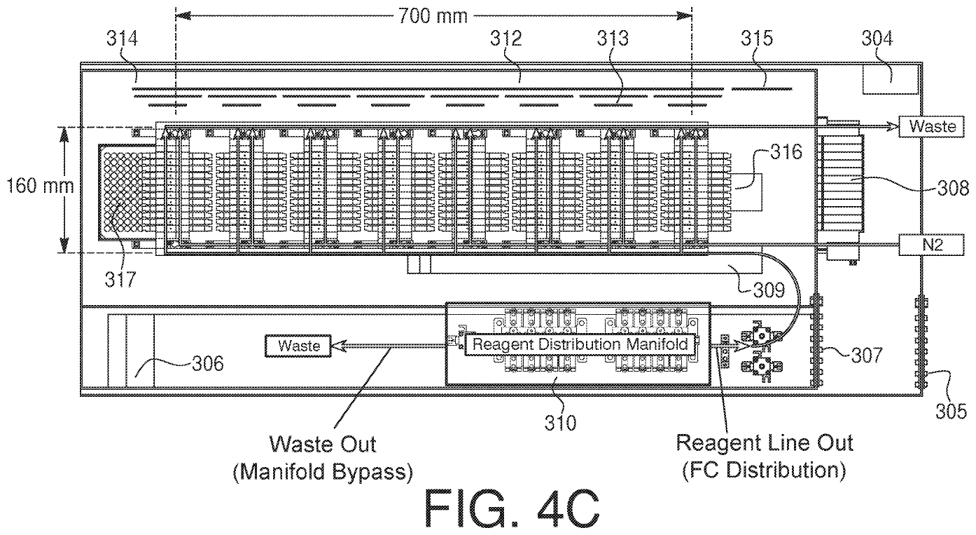

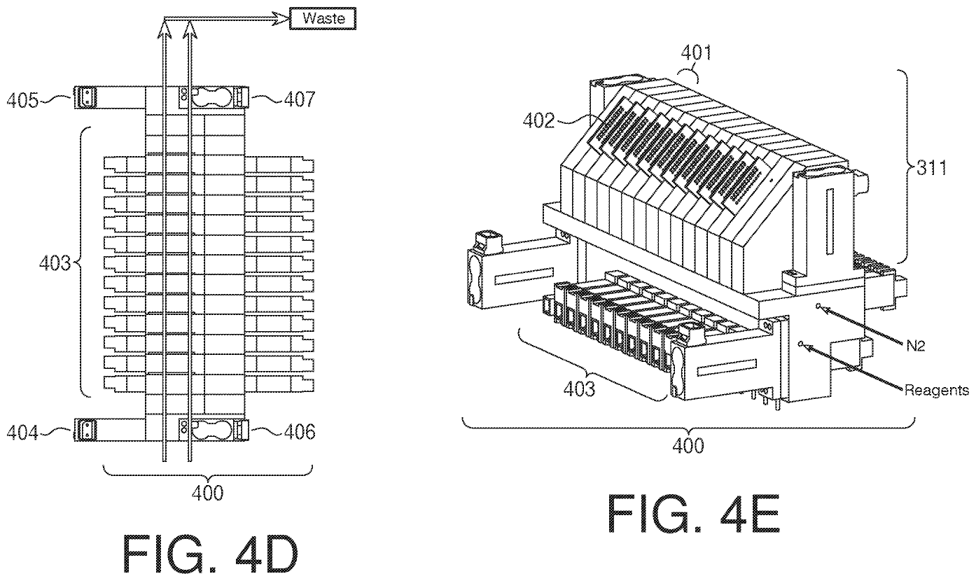

A storage module (or unit) may be configured to receive extracted biomolecules. In some instances, a storage unit receives instructions for storage location of biomolecules from a specific synthesis unit 303, flow cell block 311, or flow cell 401. A storage unit in some instances comprises a tray or surface for biomolecule storage 317. In some instances, a storage unit receives instructions for operating a drying unit. As output, a storage unit in some instances provides stored biomolecules in the form of a chamber (or container) such as a tray, plate, tape, capsules, or any combination thereof. A storage in some instances receives instructions for sealing biomolecules in a tray, plate, tape, or capsule. In some instances a storage unit provides as output metadata specific to one or more chambers. In some instances a storage unit provides as output metadata specific to one or more capsules. In some instances a storage unit provides as output metadata specific to one or more trays. A storage unit in some instances is configured to perform steps of FIG. 1B. A storage unit in some instances comprises one or more robotic stages 316 to perform steps of one or more of loading/initializing trays, moving trays to a synthesis unit/extraction unit, intermediate tray placement and movement, centrifuging trays in cold or vacuum conditions, moving trays to an encapsulation station, and moving trays to a storage module (e.g., FIG. 4A). In some instances a storage unit comprises a stage 316 configured to move one or more storage chambers to different flow cell positions at the synthesis unit. In some instances a storage unit comprises multiple redundant data copies stored in the same plate across multiple locations or in the same location (e.g., a well). In some instances one or more storage units are configured in a RAID-type storage configuration. In some instances, instances a RAID 0, RAID 1, RAID 5, RAID 6, or RAID 10 configuration is used. In some instances, configurations comprise one or more of striping, mirroring, and parity (distributed or dual parity), including any combination thereof. In some instances, a storage unit comprises a plate. In some instances, a plate comprises a plurality of wells. In some instances, the number of wells is proportional based on the number of flow cell blocks or flow cells. In some instances, a storage device comprises a well corresponding to each flow cell in a flow cell block. In some instances, a storage module comprises a deprotection module. In some instances, a deprotection module is configured to perform the operations shown in FIG. 2B, including de-sealing, incubating, and sealing. In some instances, a storage module comprises a dry down module. In some instances, a dry down module comprises a vacuum/centrifuge, evaporator, or other system for drying biomolecules.

A quality control unit may be used to evaluate synthesized biomolecules. In some instances, a quality control unit comprises one or more spectroscopic measurement devices, sequencers, or other system for evaluating biomolecules. In some instances, a quality control unit receives as input one or more extracted biomolecules. In some instances, the quality control unit provides as output a log file comprising biomolecule information. In some instances, biomolecule information comprises sequence, concentration, amount, or other information.

### Controllers

The devices, systems, or platforms provided herein for biomolecule extraction may be integrated in a data storage system. A system for data storage can comprise one or more modules. In some instances, the some or all of the one or more modules are in communication. In some examples, some or all of the one or more modules are in communication to allow transferring of polynucleotides between them. In some examples, some or all of the one or more modules are fluidically coupled. In some examples, some or all of the one or more modules are fluidically coupled with one or more tubes. A fluid may generally refer to one or more liquids used in various processes involved in handling polynucleotides, including, without limitation, synthesis, amplification, preparation for sequencing, and sequencing. In some examples, some or all of the modules are in communication to allow transferring of control commands between modules of the system. In some examples, some or all of the one or more modules are electronically coupled. A module in the system can comprise, without limitation, a synthesizer unit, an amplification chamber, a sequencer unit, a storage unit, a controller, a robotic system, or any combination thereof. In some examples, a module can further comprise a fluid source, a database or a file system, or both. In some examples, the database or file system keeps track of the storage capacity of the system. For example, the database or file system can keep track of available racks (or trays), slots (for capsules), or both. In some examples, the database or the file system is used to determine the disposition of the rack within the storage system. In some instances, movement of polynucleotides between one or more modules of a system is accomplished by one or more tubes or a robotic system. In some examples, the database or the file system is used to direct the robotic system to the correct position in the storage system. In some instances, the system is autonomous. A controller system in some instances is configured to perform the operations between modules is described in FIG. 1C.

A system for storing information can comprise a device or assembly for storing information, such as a device comprising a plurality of compartments, a base, plate, and/or a cover plate, as described further herein. The system can further comprise a material deposition system comprising a dispenser as described herein. The dispenser may be used to deposit a plurality of polynucleotides to a compartment of the plurality of compartments. The dispenser may be used to deposit a reagent for processed described herein, such as synthesis, amplification, and/or sequencing of polynucleotides. The system can further comprise one or more temperature control systems. The one or more temperature control systems may be used to heat or cool one or more components of the device or system, for example, the base plate, the cover plate, or a plurality of compartments or a portion thereof. The system can further comprise a computing system described herein. The computing system may comprise at least one processor and instructions executable by the at least one processor to perform one or more operations. In some instances, the one or more operations comprises: orchestrating movement of one or more components of the system and/or monitoring a property of the one or more components of the system. In some examples, orchestrating the movement of one or more components of the system comprises positioning the cover plate over the base plate, placing the cover plate on the base plate, or both. In some examples, orchestrating the movement of one or more components of the system comprises aligning the dispenser of the material deposition system to a compartment of the plurality of compartments. In some examples, the property comprises temperature, humidity, pressure, salinity, light sensor, UV, O₂, or any combination thereof. In some examples, monitoring the property of one or more components comprises monitoring the temperature of a component of the device in the system, such as the polynucleotide storage device comprising the base plate, the cover plate, or the plurality of components containing the polynucleotides.

A data storage system may comprise a control system. A control system may generally refer to a framework to coordinate operations between protocols, connections, modules, and devices, so they may be executed properly and on schedule. In some embodiments, the operations may be executed with one or more logic elements comprising a programmable logic controller (PLC), programable logic array (PLA), programmable array logic (PAL), generic logic array (GLA), complex programmable logic decide (CPLD), field programable gate array (FPGA), or application-specific integrated circuit (ASIC). The control system may comprise one or more network communication protocols that may be standard network communication protocols, non-standard network communication protocols, or any combination thereof. In some embodiments, the standard network communication protocols are process field bus (Profibus), process field net (Profinet), highway addressable remote transducer (HART), distributed network protocol (DNP3). Modbus, open platform communication (OPC), building automation and control networks (BACnet), common industrial protocol (CIP), or ethernet for control automation technology (EtherCAT), PCI, NVME, SAS, SATA, fiber channel, or USB. A data storage system may include industrial, manufacturing, or processing facilities. Such facilities may support objectives on a mass-scale, such as synthesizing, storing, or retrieving information stored in biomolecules. A data storage system may comprise one or more of PLCs, remote terminal units, intelligent electronic devices, engineering workstations, human machine interfaces (HMIs), data historians, communication gateways, and front-end processors. In some embodiments, a data storage system may have different controllable states as steps of a process. In some embodiments, a data storage system may use an open communication protocol. In some instances logic elements comprise one or more of a Mother1 312, Mother2 314, FPGA 313, and control 315 (FIGS. 3B-3C).

Logic elements may be used to control the operation of a data storage unit, including synthesis unit, storage unit, rack interface, or other component of a data storage unit. In some instances, logic elements are used to control the function of one or more flow cell blocks. In some instances, logic elements are used to control the function of one or more flow cells. In some instances, at least 2, 4, 6, 8, 10, 12, 16, or at least 24 flow cells are controlled per logic element. In some instances, about 2, 4, 6, 8, 10, 12, 16, or about 24 flow cells are controlled per logic element.

A non-limiting example of a system for data storage is illustrated in FIG. 8, with a feedback loop. A feedback loop may generally comprise a user 1805 that can interact with a system via a controller 1835 (e.g., PLC), for example, through a human-machine interface (HMI). The HMI may be a user interface (e.g., GUI) that connects a person or a user to one or more components (e.g., equipment, network, etc.) in the system. In some instances, a user may send an input, for example, as a query, to the controller 1835, regarding the state or function of components of the system. In some instances, the query is related to an item of information, whole or in part, that is stored in the system, such as the location, duration, or metadata of the information stored in the system.

The controller 1835 may send an output to a user 1805. The output can comprise a response to the query, which can be provided through a HMI and may be displayed on the user interface. In some instances, an interface comprises S3/OCI or cloud attach. In some instances, the controller 1835 sends status information regarding components of the ICS to the HMI and it is provided to the user 1805. In some instances, the controller 1835 implements control strategies using a system comprising a microprocessor for managing components in the system.

In some cases, the components may be a physical device, such as equipment in the system. The physical devices can be a device employed for storage or retrieval information in biomolecules. For example, physical devices can be part of a synthesizer unit 1810, storage unit 1815, amplification unit 1820, or sequencer unit 1825. In some examples, physical devices comprise one or more components illustrated in FIGS. 2-6. In some examples, physical devices comprise a robotic system 1830, which can be used for transferring or handling biomolecules in the system. In some cases, the equipment may be on-site or remote. In some examples, the controller 1835 controls a physical device or a plurality thereof, such as control motors, valves, switches, etc., in the system.

A controller 1835 may control a physical device based on one or more measurements obtained from sensors in the system. In some instances, sensors are integrated into one or more modules (e.g., a synthesizer unit 1810, storage unit 1815, amplification unit 1820, or sequencer unit 1825, robotic system 1835, flow cell, or biomolecule extraction systems, etc.). In some instances, sensors determine when and how the physical device should operate. For example, the sensor may be an integrated sensor as part of a control device comprising an actuator. In some cases, the measurements may be physical measurements obtained from sensors, such as pressure, volume, temperature, humidity, torque, vacuum, motion, flow rate (e.g., fill rate or evacuation rate), angles of orientation of devices (e.g., flow cells), etc. In some cases, the sensor is a standalone sensor. In further instances, the controller 1835 receives commands for the physical device to perform functions (e.g., pump actuation, stirrer operation, conveyor belt operation, etc.) from a user 1805, for example through a HMI.

### Sensors

The data from operations or sensors in the system, as described herein, may be fed into one or more software modules for analyzing data in the storage system. For example, the data may be sensor data from one or more compartments or modules in the system, and an algorithm may be used to monitor one or more parameters. In some examples, the algorithm monitors patterns in the sensor data and can be used to detect anomalies, for example, irregular sensor data from one or more compartments and optionally, alert a user through a HMI. As another example, the data may be an item of information or sequencing data and an algorithm may be used to convert the data to another format (e.g., convert an item of information to a nucleic acid sequence, or vice versa). In some examples, the algorithm comprises an error correction scheme that can be used to correct errors that have occurred during processes in the data storage system. In some in instances, sensors detect one or more of pressure, temperature, humidity, salinity, light, UV, O₂, flow rate, temperature gradient, electrical connections and status, and synthesis feedback data.

### Synthesis Unit

A system for data storage may comprise a synthesizer unit 1810 of FIG. 8. A synthesizer unit can be used to synthesize biomolecules, such as a plurality of polynucleotides, encoding digital information. In some instances, the system comprises more than one synthesizer units 1810. Polynucleotides may be synthesized using a method provided herein or any other suitable synthesis method known in the art. The fluidic and/or electronic control of polynucleotide synthesis in the synthesizer unit 1810 may be performed by a controller 1835. In some instances, the electronics in the synthesizer unit 1810 are in communication with the controller 1835. In some instances, the synthesizer unit 1810 has an input for receiving DNA sequences. In some instances, the synthesizer unit 1810 has an input for receiving fluids for polynucleotide synthesis. In some instances, the synthesizer unit 1810 has an output for eluting synthesized polynucleotides. In some instances, the synthesized polynucleotides are transferred to another component of the system, such as, by way of non-limiting example, a storage unit, an amplification chamber, or a sequencing unit. Synthesis units in some instances comprises one or more of a computer controller (312-315); a flow cell block 311; an extraction stage 316; a storage unit 317; a robotic interface; and a rack interface.

### Reservoirs

A data storage unit can comprise a reservoir or one or more reservoirs. In some instances, the reservoir is connected or connectable to a flow cell (via a flow cell block). In some instances, the flow cell is oriented at one or more angles (e.g., FIG. 5C). In some examples, the flow cell is at an orientation such that α>0°, β>0°, or both. In some examples, the flow cell is at an orientation such that α≤90°, β≤45°, or both. In some examples, the flow cell is at an orientation such that 0°<α≤90°, 0°<β≤45°, or both. In some examples, the flow cell is at an orientation such that a planar surface of the cavity is substantially parallel to a body force, such as gravity. In some instances, the reservoir and the flow cell are stable associated using a base station, platform, or any other suitable equipment (e.g., mounting equipment such as a flow cell block). In some instances, the reservoir is part of a fluid dispensing assembly that can be employed to dispense fluids (e.g., water, aqueous media, organic solvents, ionic liquids and the like). In some instances, the system comprises a plurality of reservoirs, each comprising a different fluid. In some instances, the reservoir comprises a liquid for extracting the plurality of biomolecules from the substrate. In some instances, the system may have a plurality of reservoirs, each comprising a liquid for biomolecule synthesis, storage, or retrieval (e.g., water, IPA, TBA (tert-butylamine), etc.). It shall be understood by one of ordinary skill in the art that the size of a reservoir comprising a fluid may be readily adjusted. The size of a reservoir may be, but is not limited to, about 10 mL, 25 mL, 50 mL, 75 mL, 100 mL, 250 mL, 500 mL, 750 mL, 1 L, 1.25 L, 1.5 L, 1.75 L, or about 2 L. In some examples, the size of the reservoir is about 10 mL to 2 L, 10 mL to 500 mL, 10 mL to 100 mL, 50 mL to 1 L, 50 mL to 500 mL, 50 mL to 100 mL, 100 mL to 2 L, 100 mL to 1 L, or 100 mL to 500 mL. Reservoirs in some instances are internal to the data storage unit or rack unit. In some instances, reservoirs are external to the data storage unit or rack unit. In some instances, the size of an external reservoir is about 10 L to 2 L, 10 L to 500 L, 10 L to 100 L, 50 L to 1 L, 50 L to 500 L, 50 L to 100 L, 100 L to 2 L, 100 L to 1 L, or 100 L to 500 L. In some instances, reservoirs are integrated into a storage unit.

In some instances, the fluid dispensing assembly comprises a pump for moving fluid to or from a reservoir or a plurality of reservoirs. In some instances, the fluid dispensing assembly comprises a manifold 310, a valve assembly, or both. In certain aspects, the assembly comprises a mechanism for delivering predetermined quantities of fluid to the flow cell. The fluids in some instances are dispensed by a pumping mechanism. A standard pumping technique for pumping fluids known in the art may be employed in the system. Non-limiting examples of pumping comprises means of a peristaltic pump, a pressurized fluid bed, a positive displacement pump. e.g., a syringe pump, and the like. In some examples, the system additionally comprises heating and/or cooling elements and/or insulating elements for controlling the temperature within various fluid reservoir(s), the flow cell, or within the mechanisms for transferring the fluid between the reservoir(s) and flow cell (e.g., manifold), or any combination thereof. In some instance a valve bank 306 is used to control movement of fluids. In some instances, a fluid manifold 310 is used to distribute and control movement of fluids. In some instances, a manifold 310 comprises one or more valves.

In some examples, a manifold 310 connects or is connectable to (directly or indirectly) one or more reservoirs. In this way, different fluid reagents can be contacted to a substrate in the flow cell. In some examples, reagents for performing different steps in the synthesis of a biomolecule (e.g., a nucleic acid or polypeptide) is introduced sequentially into the flow cell.

The system can comprise a top manifold and a bottom manifold. In some examples, a top manifold and bottom manifold connect to a same one or more reservoirs. In some examples, a top manifold and bottom manifold connect to a different one or more reservoirs. The reservoirs in some instances comprise: a waste reservoir, a sample collection reservoir, or one or more reservoirs, each comprising a different fluid. In some examples, a top manifold and a bottom manifold both connect to a waste reservoir. In some examples, the top manifold, the bottom manifold, or both, comprise a separate waste reservoir. In some examples, the bottom manifold is connected to a sample collection reservoir. In some examples, a top manifold is connected to a sample collection reservoir. In some examples, the bottom manifold is connected to one or more reservoirs, each comprising a different fluid. In some examples, a top manifold is connected to one or more reservoirs, each comprising a different fluid. In some instances, the top manifold connects, or is connectable to, a pump for displacing fluid from the flow cell.

Each of the manifolds may be independently controlled. In some instances, each manifold comprises a plurality of valves that can open or close paths between components of the system. In some instances, fluid passing through the top manifold, bottom manifold, or both can be independently controlled. e.g., through the use of automatically or manually operated valves. In some instances, the fluid passing through the system is controlled via a controller, which is coupled to one or more actuators that open and close valves connected to the flow cell, reservoir, or pump. In such instances, the controller is used to control the amount or rate of fluid or gas flow throughout the system.

In some instances, the system comprises a top manifold that communicates with the portion of the flow cell comprising at least one top opening. In some examples, the top manifold comprises a conduit, which communicates with the flow cell comprising at least one top opening. In some instances, the top manifold comprises at least one opening that connect with or are coextensive with the at least one top opening of the flow cell. In some instances, the system comprises a bottom manifold that communicates with the portion of the flow cell comprising at least one bottom opening. In some examples, the bottom manifold comprises a conduit, which communicates with the flow cell comprising at least one bottom opening. A conduit or tube connection one or more components of the system, in some instances comprises by way of non-limiting example, PFA, however, may be any suitable material known in the art. Conduit (or routing), in some instances comprises but is not limited to, about ¼″, ⅛″. 1/16″, or 1/32″ in diameter. In some instances, the bottom manifold comprises at least one opening that connect with or are coextensive with the at least one bottom opening of the flow cell. In some instances, when the flow cell is in operation, the top manifold is distal to a surface on which the portion of the flow cell comprising the at least one bottom opening is situated. In some examples, the top manifold can be used to introduce or backfill fluid into a fully charged flow cell.

In some instances, the system comprising the flow cell comprises a plurality of top and bottom submanifolds, which allow fluid (liquid or gas) to into the flow cell by a common top and bottom conduit respectively. In some examples, the plurality of top and bottom submanifolds are connected to one or more separate dispensing lines. In still other aspects, a top and bottom submanifold can be coupled via a common dispensing line, however fluid through the top or bottom manifold can be independently controlled by appropriately placed valves (e.g., through a controller).

The system may further comprise a vacuum source. In some instances, the vacuum source is connected to or in communication with the flow cell. In some instances, the system comprises one or more electronic sensors, mechanical sensors, or both that sense conditions of the flow cell. In some instances, a controller as described herein is programed to regulate flow of fluids in the system through the one or more sensors. In some examples, the system comprises a fluid level sensor, one or more pressure transducers, one or more pressure regulators, manually or automatically operated valves and/or pumps.

The system can further comprise a mechanisms for facilitating movement of a substrate into and out of a cavity of a flow cell, as described herein. In a non-limiting example, a system can comprise a lift mechanism for placing a substrate into the cavity of a flow cell and/or lifting the substrate out of the cavity of the flow cell in a controlled manner, e.g., manually or in an automated fashion.

### Flow Cell Block (FC Block)

In some instances, a synthesis unit comprises a flow cell block 311. In some instances, a flow cell block arrangement is shown in FIGS. 4D-4E. In some instances, a flow cell block comprises a plurality of flow cells 401. In some instances, a flow cell comprises one or more solid supports 402 for biomolecule synthesis (e.g., polynucleotides). In some instances, a flow cell block 311 comprises an inlet 404 and outlet 405 for reagents and waste, respectively. In some instances, a flow cell block comprises an inlet 406 and outlet 407 for gases (e.g., nitrogen or argon). In some instances ports (or inlets/outlets) are controlled by one or more valves. In some instances, a synthesis unit comprises about 4, 5, 6, 7, 8, 9, 10, 11, 12, 14, 16, 18, 20, 24, 32, 48, 64, 96, 128, 256, 512, or about 1024 flow cell blocks. In some instances, synthesis unit comprises at least 4, 5, 6, 7, 8, 9, 10, 11, 12, 14, 16, 18, 20, 24, 32, 48, 64, 96, 128, 256, 512, or at least 1024 flow cell blocks. In some instances, a synthesis unit comprises no more than 4, 5, 6, 7, 8, 9, 10, 11, 12, 14, 16, 18, 20, 24, 32, 48, 64, 96, 128, 256, 512, or no more than 1024 flow cell blocks. In some instances, a synthesis unit comprises 4-24, 4-20, 4-16, 4-12, 4-8, 6-24, 6-16, 6-12, 6-10, 8-24, 8-16, 8-12, 10-24, 12-24, or 16-24 flow cell blocks. In some instances, a flow cell block comprises: one or more flow cells comprising: one or more solid supports configured for polynucleotide synthesis; and one or more ports for exchange of gases, synthesis reagents and/or extracted polynucleotides. In some instances, a flow cell block comprises a plurality of valves 403. In some instances, each valve controls fluid flow into each flow cell 401. In some instances, each flow cell 401 comprises at least one solid support 402. In some instances, each solid support 402 is double sided or single sided. Flow cell blocks may comprise different arrangements of flow cells. In some instances, flow cells are placed nearly vertically in the flow cell block (e.g., α=90° as shown in FIG. 5C). In some instances, flow cells are placed at an angle relative to the x or y planes (e.g., FIG. 4E). In some instances, a flow cell block further comprises a plurality of connectors and/or valves configured for reagent or gas distribution to individual flow cells. In some instances, a flow cell block comprises an inlet and outlet for each flow cell. In some instances, a flow cell block comprises an inlet and outlet for each flow cell for liquids. In some instances, a flow cell block comprises an inlet and outlet for each flow cell for gases (e.g., FIG. 4D). Flow cells blocks in some instances comprise at least one manifold. In some instances, a manifold is shown in FIGS. 4B-4C. In some instances, a manifold comprises a reagent distribution manifold. In some instances, at least one reagent distribution manifold is connected to one or more of a gas supply, flow waste, supply prime waste, and supply inlet.

In some instances, flow cells 401 are spaced inside a synthesis unit. In some instances, a flow cell block 311 comprises a spacing of at least 10, 25, 50, 75, 100, 125, 150, 175, 200, or at least 250 mm. In some instances, a flow cell block comprises a spacing of no more than 10, 25, 50, 75, 100, 125, 150, 175, 200, or no more than 250 mm. In some instances, a flow cell block comprises a spacing of 10-300, 10-250, 10-225, 10-200, 10-175, 10-150, 10-125, 10-100, 10-75, 10-50, 25-300, 25-275, 25-250, 25-200, 25-175, 25-150, 25-125, 25-100, 50-300, 50-250, 50-275, 50-225, 75-300, 75-250, 75-200, 75-150, 75-126, 75-175, 100-300, 100-250, 100-200, 150-300, 200-500, or 250-300 mm.

Any number of combinations of flow cell blocks 311 and flow cells per block may be used for a synthesis unit. In some instances, a synthesis unit comprises at least 12, 24, 36, 48, 64, 72, or at least 96 flow cells. In some instances, a synthesis unit comprises about 12, 24, 36, 48, 64, 72, or about 96 flow cells. In some instances, a synthesis unit comprises no more than 12, 24, 36, 48, 64, 72, or no more than 96 flow cells. In some instances, a synthesis unit comprises 4-128, 4-96, 4-64, 8-128, 8-96, 8-96, 8-64, 16-256, 16-128, 16-96, 16-72, 16-64, 24-256, 24-128, 24-96, 48-256, 48-128, 64-256, 64-128, 64-96, 72-128, or 72-256 flow cells.

Flow cells 401 within a flow cell block 311 may comprise different pitches. In some instances, flow cells comprise a pitch distance of no more than 1, 2, 5, 7, 8, 9, 10, 11, 12, 16, 20, or no more than 25 mm. In some instances, flow cells comprise a pitch distance of at least 1, 2, 5, 7, 8, 9, 10, 11, 12, 16, 20, or at least 25 mm. In some instances, flow cells comprise a pitch distance of about 1, 2, 5, 7, 8, 9, 10, 11, 12, 16, 20, or about 25 mm. In some instances, flow cells comprise a pitch distance of 1-20, 1-15, 1-12, 1-11, 1-10, 1-9, 2-20, 2-15, 2-12, 2-10, 5-20, 5-15, 5-12, 5-10, 5-9, 7-15, 7-20, 7-25, 8-20, 8-15, 9-15, 9-20, 12-20, or 12-15 mm.

### Flow Cells

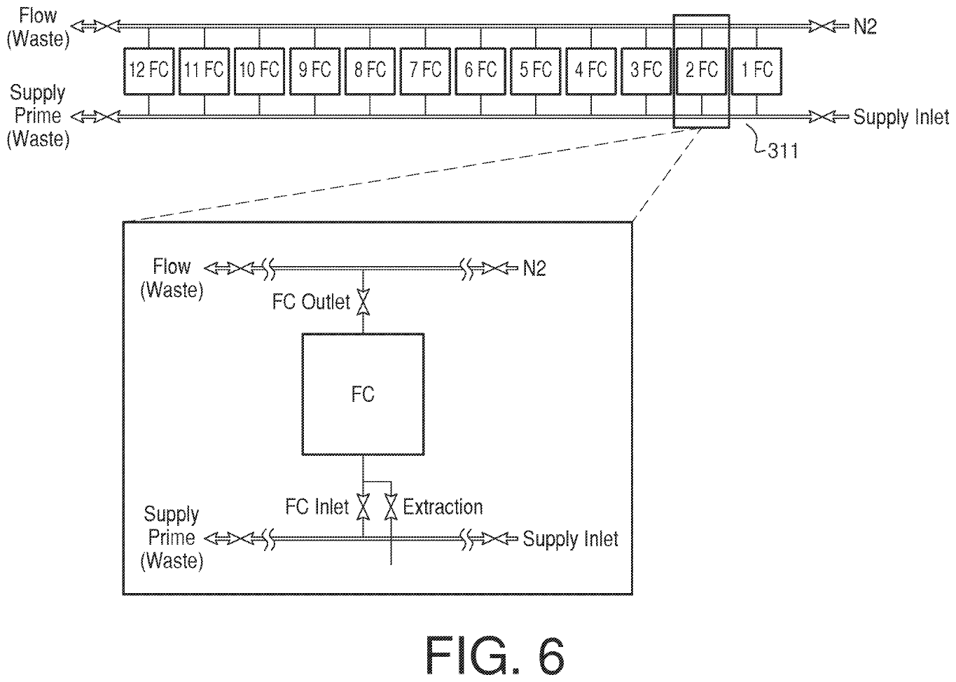

A flow cell 401, or a system or platform comprising a flow cell (e.g., with valves 400), may be connected to or coextensive with one or more components of the system, such as the synthesizer unit 1810, storage unit 1815, amplification unit 1820, or sequencer unit 1825. In some instances, the flow cell as provided herein is used for biomolecule synthesis, biomolecule extraction, or both. In alternative instances, the synthesized polynucleotides can be transferred to a flow cell or a system comprising a flow cell for extraction of biomolecules from a synthesis surface, whole or in-part. In some examples, once the polynucleotides are cleaved from the surface, they are collected and transferred to another component of the system, such as, by way of non-limiting example, storage unit 1815, amplification unit 1820, or sequencer unit 1825. In some instances, the flow cell is oriented to maximize the recovery of liquid from the flow cell, for example, by adjusting angles α or β as defined herein (e.g., FIG. 5C). Further, an apparatus comprising at least one logic element for performing one or more operations of a biomolecule synthesis and/or extraction platform, as provided herein may be in communication with or may be part of a controller 1835 of a larger data storage system. In some instances a flow cells comprises an input and an output port. In some instances, the output port is configured to direct liquids to waste or an extraction stage. In some instances, a flow cell is depicted as shown in FIG. 2A. An exemplary schematic showing fluidics routing for a flow cell block and individual flow cell is shown in FIG. 6. In some instances a flow cell comprises an inlet and outlet. In some instances, a flow cell inlet also comprises a bypass for transfer to an extraction stage. In some instances, a flow cell comprises an inlet for liquid reagents. In some instances, a flow cell comprises an inlet for gases. In some instances, a gas outlet is connected to a flow cell outlet and a waste outlet. In some instances, fluidic movement through the flow cell is accomplished by positive pressure on the inlet. In some instances, fluidic movement through the flow cell is accomplished by vacuum on the outlet.

In some instances, the flow cell 401 comprises a housing defining a flow chamber. i.e., a cavity. In some instances, the flow cell comprises an opening for receiving a substrate. The substrate can comprise a biomolecules, such as polynucleotides or proteins. In some instances, the cavity can enclose a substrate and fluidically sealed. In some examples, the opening of the flow cell can be configured to be sealable after the array substrate is placed therein, to prevent the leakage of fluids from the flow cell through the opening. Such seals may include a flexible material that is sufficiently flexible or compressible to form a fluid tight seal that may be maintained under increased pressures encountered in the use of the device. The flexible member may be, for example, rubber, flexible plastic, flexible resins, and the like and combinations thereof. In one aspect, the flexible material is substantially inert with respect to the fluids introduced into the device and docs not interfere with the reactions that occur within the device. The flexible member may be a gasket and may be in any shape such as, for example, circular, oval, rectangular, and the like, e.g., the flexible member may be in the form of an O-ring (e.g., FFKM Kalrez O-ring) in certain embodiments.

In some instances, the flow cell 401 can comprise two halves. In some examples, the two halves can be brought into proximity to enclose a substrate and fluidically seal the substrate. In some examples, when the flow chamber comprises two halves, the halves may be stably associated by providing mating elements (e.g., a prong on one half that fits into an opening of another half). However, in another aspect, the two halves may be stably associated by clamps or other pressure sealing mechanisms. In one aspect, the two halves are sealable and engaged during reaction steps (e.g., synthesis steps) and are separable at other times to permit the support to be placed into and removed from the chamber of the flow cell. Movement of the one half with respect to the other may be achieved by means of, for example, pistons, and so forth. The movement may be controlled electronically by means that are conventional in the art. In some instances, two halves of a flow cell are joined mechanically. In some instances, two halves of a flow cell are joined using one or more gaskets. In some instances, two halves of a flow cell are joined using a bonding agent. In some instances, a bonding agent comprises an epoxy, a polyurethane, or other adhesive.

The dimension of the flow cell 401, the cavity therein, or both can be varied. In some instances, the dimension of flow cell, the cavity, or both is varied depending on the dimension of the substrate that is placed in the flow cell. In some instances, the substrate comprises an array on to which a chemical compounds is synthesized. In some examples, the cavity has a first length and a second length (e.g., length and width). In some examples, the cavity is substantially rectangular. In some instances, a first length, a second length, or both, of cavity is about 15 to 22 mm. In some instances, a first length, a second length, or both, of cavity is about 15 to 16, 15 to 17, 15 to 18, 15 to 19, 15 to 20, 15 to 21, 15 to 22, 16 to 17, 16 to 18, 16 to 19, 16 to 20, 16 to 21, 16 to 22, 17 to 18, 17 to 19, 17 to 20, 17 to 21, 17 to 22, 18 to 19, 18 to 20, 18 to 21, 18 to 22, 19 to 20, 19 to 21, 19 to 22.20 to 21, 20 to 22, or 21 to 22 mm. In some instances, a first length, a second length, or both, of cavity is about 15, 15.5, 16, 16.5, 17, 17.5, 17.9, 18, 18.5, 19, 19.5, 19.9, 20, 20.5, 21, 21.5, or 22 mm. In some instances, a first length, a second length, or both, of cavity is at least about 15, 15.5, 16, 16.5, 17, 17.5, 18, 18.5, 19, 19.5, 20, 20.5, 21, or 21.5 mm. In some instances, a first length, a second length, or both, of cavity is at most about 15.5, 16, 16.5, 17, 17.5, 18, 18.5, 19, 19.5, 20, 20.5, 21, 21.5, or 22 mm. In some instances, the first length of the cavity is about 17.9 mm and the second length of the cavity is about 19.9 mm. In some instances, the cavity comprises a third length (e.g., height). In some examples, the cavity is formed in a recess in the body of the flow cell. In some examples, a third length of a cavity is about 0.1 to 0.5 mm. In some instances, a third length of cavity is about 0.1 to 0.15, 0.1 to 0.2, 0.1 to 0.25, 0.1 to 0.3, 0.1 to 0.35, 0.1 to 0.4, 0.1 to 0.45, 0.1 to 0.5, 0.15 to 0.2, 0.15 to 0.25, 0.15 to 0.3, 0.15 to 0.35, 0.15 to 0.4, 0.15 to 0.45, 0.15 to 0.5, 0.2 to 0.25, 0.2 to 0.3, 0.2 to 0.35, 0.2 to 0.4, 0.2 to 0.45, 0.2 to 0.5, 0.25 to 0.3, 0.25 to 0.35, 0.25 to 0.4, 0.25 to 0.45, 0.25 to 0.5, 0.3 to 0.35, 0.3 to 0.4, 0.3 to 0.45, 0.3 to 0.5, 0.35 to 0.4, 0.35 to 0.45, 0.35 to 0.5, 0.4 to 0.45, 0.4 to 0.5, or 0.45 to 0.5 mm. In some instances, a third length of cavity is about 0.1, 0.15, 0.2, 0.25, 0.3, 0.35, 0.4, 0.45, or 0.5 mm. In some instances, a third length of cavity is at least about 0.1, 0.15, 0.2, 0.25, 0.3, 0.35, 0.4, or 0.45 mm. In some instances, a third length of cavity is at most about 0.15, 0.2, 0.25, 0.3, 0.35, 0.4, 0.45, or 0.5 mm. In some instances, the flow cell is a Hele-Shaw flow cell, since the height of the cavity is much smaller in comparison to the length and the width. In some instances, the dimensions of the substrate are less than the dimensions of the cavity. In some instances, the dimension of the flow cell is greater than the dimensions of the cavity.

The volume containable by the cavity of the flow cell 401 can vary. In some instances, the volume in the cavity is about 22 to about 242 mm³. In some instances, the volume in the cavity is about 50 to about 200 mm³. In some instances, the volume in the cavity is about 22 to 45, 22 to 53, 22 to 68, 22 to 89.22 to 95, 22 to 115, 22 to 125, 22 to 145, 22 to 194, 22 to 242, 45 to 53, 45 to 68, 45 to 89, 45 to 95, 45 to 115.45 to 125.45 to 145.45 to 194.45 to 242.50-200, 53 to 68, 53 to 89, 53 to 95, 53 to 115, 53 to 125, 53 to 145, 53 to 194, 53 to 242, 68 to 89, 68 to 95, 68 to 115, 68 to 125, 68 to 145, 68 to 194, 68 to 242, 89 to 95, 89 to 115, 89 to 125, 89 to 145, 89 to 194, 89 to 242, 95 to 115, 95 to 125, 95 to 145, 95 to 194, 95 to 242, 115 to 125, 115 to 145, 115 to 194, 115 to 242, 125 to 145, 125 to 194, 125 to 242, 145 to 194, 145 to 242, or 194 to 242 mm³. In some instances, the volume in the cavity is about 22, 45, 50, 53, 68, 89, 95, 115, 125, 145, 194, 200, or 242 mm³. In some instances, the volume in the cavity is at least about 22, 45, 50, 53, 68, 89, 95, 115, 125, 145, 194, or 200 mm³. In some instances, the volume in the cavity is at most about 45, 50, 53, 68, 89, 95, 115, 125, 145, 194, 200, or 242 mm³.

The flow cell device 401 provided herein can comprise an insulating member around at least a portion of the device. The flow cell device can expose a substrate within the cavity to a flow of fluid, wherein a first end of the substrate and a second end of the substrate are exposed to a fluid comprising substantially the same composition at a given time interval. In some instances, the flow cell can be used for extracting biomolecules from a substrate. In some instances, the flow cell can be used for performing in situ synthesis of biomolecules (e.g., polynucleotides or polypeptides) on the substrate.

The flow cell 401 can have an opening (e.g., port and/or valve). In some instances, the flow cell comprises at least two openings. In some instances, the flow cell comprises first opening and a second opening, for example, an inlet and an outlet. In some instances, in operation, outlet port(s) sit vertically above inlet port(s). In some examples, the flow cell comprises 1 to 25 inlet holes, outlet holes, or both. In some instances, the flow cell comprises 1 to 2, 1 to 5, 1 to 8, 1 to 10, 1 to 12, 1 to 15, 1 to 20, 1 to 22, 1 to 25, 2 to 5, 2 to 8, 2 to 10, 2 to 12, 2 to 15, 2 to 20, 2 to 22, 2 to 25, 5 to 8, 5 to 10, 5 to 12, 5 to 15, 5 to 20, 5 to 22, 5 to 25, 8 to 10, 8 to 12, 8 to 15, 8 to 20, 8 to 22, 8 to 25, 10 to 12, 10 to 15, 10 to 20, 10 to 22, 10 to 25, 12 to 15, 12 to 20, 12 to 22, 12 to 25, 15 to 20, 15 to 22, 15 to 25, 20 to 22, 20 to 25, or 22 to 25 inlet holes, outlet holes, or both. In some instances, the flow cell comprises 1, 2, 5, 8, 10, 12, 15, 20, 22, or 25 inlet holes, outlet holes, or both. In some instances, the flow cell comprises at least 1, 2, 5, 8, 10, 12, 15, 20, or 22 inlet holes, outlet holes, or both. In some instances, the flow cell comprises at most 2, 5, 8, 10, 12, 15, 20, 22, or 25 inlet holes, outlet holes, or both. In some instances, the number and position of inlet holes, outlet holes, or both is varied to bias flow across different regions of the flow cell. However, in some instances, the number of inlet holes and outlet holes are uniform to provide for an unbiased flow. In some instances, inlet holes are uniformly spaced along the bottom of the cavity. In some instances, the outlet holes are uniformly spaced along the top of the cavity. In some instances, inlet holes are not uniformly spaced along the bottom of the cavity. In some instances, the outlet holes are not uniformly spaced along the top of the cavity. In some instances, uniform flow is attained, provided the inlet and outlet are symmetric. In some examples, uniform flow is attained, provided the inlet and outlet are symmetric, but the spacing between the inlet holes or outlet holes are not uniform.

The at least two openings of the cavity can vary in size. The at least two openings may be drilled into the housing of the flow cell. In some instances, the at least two openings are located on opposite sides of the cavity, for example in diagonal corners of a rectangular cavity, or along opposing sides of a rectangular cavity. In some instances, each of the at least two openings (e.g., inlet and outlet hole(s)) is about 100 to 500 μm. In some instances, each of the at least two openings has a diameter of about 100 to 150, 100 to 200, 100 to 250, 100 to 300, 100 to 350, 100 to 400, 100 to 450, 100 to 500, 150 to 200, 150 to 250, 150 to 300, 150 to 350, 150 to 400, 150 to 450, 150 to 500, 200 to 250, 200 to 300, 200 to 350, 200 to 400, 200 to 450, 200 to 500, 250 to 300, 250 to 350, 250 to 400, 250 to 450, 250 to 500, 300 to 350, 300 to 400, 300 to 450, 300 to 500, 350 to 400, 350 to 450, 350 to 500, 400 to 450, 400 to 500, or 450 to 500 μm. In some instances, each of the at least two openings has a diameter of about 100, 150, 200, 250, 300, 350, 400, 450, or 500 μm. In some instances, each of the at least two openings has a diameter of at least about 100, 150, 200, 250, 300, 350, 400, or 450 μm. In some instances, each of the at least two openings has a diameter of at most about 150, 200, 250, 300, 350, 400, 450, or 500 μm. In some examples, the one or more openings of the cavity are connected to one or more manifolds for feeding fluid (e.g., liquid, gas, or both) in and/or out of the cavity. In some instance, the diameter of a first opening and a second opening of the at least two openings are the same. In some instance, the diameter of a first plurality of opening and a second plurality of opening of the at least two openings are the same. In some instance, the diameter of a first opening and a second opening of the at least two openings are different. In some instance, the diameter of a first plurality of opening and a second plurality of opening of the at least two openings are different.

In some instances, high pressure at one or more openings (e.g., inlet holes) equalizes pressure in a manifold. In some instances, a manifold provides a mechanism for removing bubbles, or to reduce the presence of bubbles in the one or more openings (e.g., inlet holes). In some instances, the diameter of an outlet hole in the flow cell which connects or is connectable to a manifold is larger than the diameter of an inlet hole. e.g., at least about 1-fold larger, at least about 1.5-fold larger, at least about 2-fold larger or at least about 4-fold larger. In some instances, flow through the inlet and/or outlet holes to the manifolds is controlled by providing a valve whose opening and closing is controlled by a controller, such as a micro-processor.

In some instances, roles of one or more manifolds may be reversed between or during operation of the flow cell device. In some examples, it is advantageous to introduce a fresh reagent from the top of the flow cell (e.g., such as when the fresh reagent is less dense than the resident liquid). In some instances, a valve can be shut in a manifold (e.g., top manifold) to increase pressure in the manifold for introducing liquid into the flow cell through the outlets and the manifold (e.g., bottom manifold) can be used to vent the flow cell device.

The flow cell 401 may be placed in a vertical orientation using a stand or base. The flow cell may comprise or may be connectable to a base station or platform to which, in some instances, one or more fluid dispensing stations can be stably associated (e.g., by mounting). In some examples, a mount is used to place the flow cell on a stand or base, such that the flow cell can be oriented at one or more angles.

The flow cell 401 may be placed in a vertical orientation and/or rotated using a stand or base. The flow cell may comprise or may be connectable to a base station or platform to which, in some instances, one or more fluid dispensing stations can be stably associated (e.g., by mounting). In some examples, a mount is used to place the flow cell on a stand or base. In some instances, the stand or the base allows the flow cell to be oriented at one or more angles, such as angles α or β as described herein. In some instances, a user or an automated system (e.g., robotic system) is used to adjust or orient the flow cell at one or more angles (e.g., angles α or β). In some instances, a flow cell oriented at angles α=90° and β=45° (e.g., FIG. 5C), may comprise no more than two openings. The cavity may be substantially rectangular or square. The fluidic boundary of the cavity may be defined by the O-ring, which can fluidically seal the cavity. In some instances, the flow cell comprises a first opening at the bottom and a second opening at the top. In an exemplary embodiment, when the cavity is being filled, the liquid can enter from the first opening (bottom), and any excess liquid can be removed from the second opening (top) (see, for example. FIG. 5A “fill” or “wash”, or FIG. 5B “fill”). Further, when the cavity is being evacuated, air may be filled through the second opening (top) and the liquid in the cavity is evacuated through the first opening (bottom) (see, for example. FIG. 5A “dry” or FIG. 5B “extract”).

A flow cell as provided herein can be integrated into a system, such as system for synthesizing and/or extracting material on a surface (e.g., FIGS. 4A-4E). In some instances, the system extracts biomolecules, such as polynucleotides, from a substrate. In some instances, the flow cell comprising a cavity is oriented such that a planar surface of the cavity is substantially parallel to a body force, such as gravity, in the system. In some examples, a substrate comprising a plurality of biomolecules is placed within the cavity of the substrate and fluidically sealed (e.g., using an O-ring). Flow cells may comprise a heating or cooling unit configured to change the temperature of the flow cell. In some instances, a flow cell comprises a heating unit. In some instances, a flow cell comprises a cooling unit. In some instances, a flow cell comprises a controller which regulates the flow cell temperature. In some instances, a flow cell is heated during deprotection or extraction steps. In some instances the heating or cooling unit is configured to modulate the temperature of a plurality of flow cells, such as a flow cell block.

### Solid Supports

A synthesizer unit may comprise one or more solid supports. In some instances, a solid support 402 is shown in FIG. 4E. In some instances, a solid support comprises a chip. The solid support may comprise a surface for polynucleotide synthesis. In some instances, a flow cell comprises a plurality of solid supports. In some instances, a solid support is in fluid communication with a flow cell. In some instances, a solid support comprises a plurality of loci for synthesis of biomolecules. In some instances, loci are addressable. In some instances, different biomolecules are synthesized at each addressable locus. In some instances, biomolecules comprise polynucleotides. In some instances, polynucleotides comprising different sequences are synthesized at each addressable locus. Control over individual loci in some instances comprises masking. In some instances, masking comprises control of reagent contact with one or more loci on the surface. In some instances reagents comprise deprotection reagents, coupling reagents, or other reagent used in polynucleotide synthesis. In some instances, a CMOS device is used to control synthesis at each locus on the surface. Use of masking at each locus in some instances allows washing of reagents (a coupling reagent, bulk reagent, wash solvent, or other reagent) over an entire surface. In some instances, masking controls which loci are allowed to react with a specific reagent. In some instances, biomolecules are synthesized on both sides of a solid support.

Different arrangements of solid supports may be used inside a flow cell. In some instances, a flow cell comprises a plurality of solid supports. In some instances, a flow cell comprises at least 10, 20, 30, 50, 70, 100, 120, 140, 160, 180, 200, 220, 240, 260, 280, 300, or at least 500 solid supports for polynucleotide synthesis. In some instances, synthesis occurs on two surfaces (e.g., front and back) of a solid support. In some instances, a flow cell comprises at least 10, 20, 30, 50, 70, 100, 120, 140, 160, 180, 200, 220, 240, 260, 280, 300, or at least 500 surfaces for polynucleotide synthesis. In some instances, a flow cell comprises about 10, 20, 30, 50, 70, 100, 120, 140, 160, 180, 200, 220, 240, 260, 280, 300, or about 500 surfaces for polynucleotide synthesis. In some instances, a flow cell comprises 10-500, 25-500, 50-500, 100-500, 150-500, 100-300, 150-250, 200-300, 200-500, or 300-500 surfaces for polynucleotide synthesis. In some instances, a flow cell comprises 5-10, 5-15, 5-20, 5-25, 10-25, 10-50, or 20-50 solid supports wide by 5-10, 5-15, 5-20, 5-25, 10-25, 10-50, or 20-50 solid supports high.

Solid supports may be constructed from various materials. In some instances, the solid support, the surface, or both comprise a material described herein. In some instances, the material comprises a metal or organic polymer. In some instances, the material comprises steel (e.g., stainless steel) or other metal alloy. In some instances, the material comprises polyethylene, polypropylene, or other polymer. In some instances, the structure comprises a flexible material, such as those provided herein. Exemplary flexible materials include, without limitation, modified nylon, unmodified nylon, nitrocellulose, and polypropylene. In some instances, the materials comprise a rigid material, such as those provided herein. Exemplary rigid materials include, without limitation, glass, fuse silica, silicon, silicon dioxide, silicon nitride, plastics (for example, polytetrafluoroethylene, polypropylene, polystyrene, polycarbonate, and blends thereof, and metals (for example, steel, gold, platinum). In some instances, materials disclosed herein may be fabricated from a material comprising silicon, polystyrene, agarose, dextran, cellulosic polymers, polyacrylamides, polydimethylsiloxane (PDMS), glass, or any combination thereof. In some examples, materials disclosed herein are manufactured with a combination of materials listed herein or any other suitable material known in the art. In some instances, a storage chamber comprises the materials described herein for solid supports.

In some instances, the solid support has varying dimensions. In some instances, a size of the solid support is between about 40 and 120 mm by between about 25 and 100 mm. In some instances, a size of the solid support is about 80 mm by about 50 mm. In some instances, a width of a solid support is at least or about 10 mm, 20 mm, 40 mm, 60 mm, 80 mm, 100 mm, 150 mm, 200 mm, 300 mm, 400 mm, 500 mm, or more than 500 mm. In some instances, a height of a solid support is at least or about 10 mm, 20 mm, 40 mm, 60 mm, 80 mm, 100 mm, 150 mm, 200 mm, 300 mm, 400 mm, 500 mm, or more than 500 mm. In some instances, the solid support has a planar surface area of at least or about 100 mm²; 200 mm²; 500 mm²; 1,000 mm²; 2,000 mm²; 4,500 mm²; 5,000 mm²; 10,000 mm²; 12.000 mm²; 15,000 mm²; 20,000 mm²; 30.000 mm²; 40,000 mm²; 50,000 mm² or more. In some instances, the thickness of the solid support is between about 50 mm and about 2000 mm, between about 50 mm and about 1000 mm, between about 100 mm and about 1000 mm, between about 200 mm and about 1000 mm, or between about 250 mm and about 1000 mm. Non-limiting examples thickness of the solid support include 275 mm, 375 mm, 525 mm, 625 mm, 675 mm, 725 mm, 775 mm and 925 mm. In some instances, the thickness of the solid support is at least or about 0.5 mm, 1.0 mm, 1.5 mm, 2.0 mm, 2.5 mm, 3.0 mm, 3.5 mm, 4.0 mm, or more than 4.0 mm.

In some instances, two or more solid supports are assembled. In some instances, solid supports are interfaced together on a larger unit. Interfacing may comprise exchange of fluids, electrical signals, or other medium of exchange between solid supports. This unit is capable of interface with any number of servers, computers, or networked devices. For example, a plurality of solid support is integrated onto a rack unit, which is conveniently inserted or removed from a server rack. The rack unit may comprise any number of solid supports. In some instances the rack unit comprises at least 1, 2, 5, 10, 20, 50, 100, 200, 500, 1000, 2000, 5000, 10,000, 20,000, 50,000, 100,000 or more than 100,000 solid supports. In some instances, two or more solid supports are not interfaced with each other. Polynucleotides (and the information stored in them) present on solid supports can be accessed from the rack unit. Access includes removal of polynucleotides from solid supports, direct analysis of polynucleotides on the solid support, or any other method which allows the information stored in the polynucleotides to be manipulated or identified. Information in some instances is accessed from a plurality of racks, a single rack, a single solid support in a rack, a portion of the solid support, or a single locus on a solid support. In various instances, access comprises interfacing polynucleotides with additional devices such as mass spectrometers. HPLC, sequencing instruments, PCR thermocyclers, or other device for manipulating polynucleotides. Access to nucleic acid information in some instances is achieved by cleavage of polynucleotides from all or a portion of a solid support. Cleavage in some instances comprises exposure to chemical reagents (ammonia or other reagent), electrical potential, radiation, heat, light, acoustics, or other form of energy capable of manipulating chemical bonds. In some instances, cleavage occurs by placing the solid support in a flow cell. In some examples, one or more orientations of the flow cell has been optimized to maximize a liquid comprising the polynucleotides that can be recovered from the flow cell (e.g., angles α or β). In some instances, cleavage occurs by charging one or more electrodes in the vicinity of the polynucleotides. In some instances, electromagnetic radiation in the form of UV light is used for cleavage of polynucleotides. In some instances, a lamp is used for cleavage of polynucleotides, and a mask mediates exposure locations of the UV light to the surface. In some instances, a laser is used for cleavage of polynucleotides, and a shutter opened/closed state controls exposure of the UV light to the surface. In some instances, access to nucleic acid information (including removal/addition of racks, solid supports, reagents, polynucleotides, or other component) is completely automated.

Solid supports as described herein comprise an active area. In some instances, the active area comprises regions, cells, features, or loci for nucleic acid synthesis. In some instances, the active area comprises regions or loci for nucleic acid storage. In some examples, the regions or loci comprise the one or more modules. In some examples, the regions or loci comprise the second one or more modules. In some instances, the regions are addressable. In some examples, the regions are addressable through an electrode.

The active area comprises varying dimensions. For example, the dimension of the active area is between about 1 mm to about 50 mm by about 1 mm to about 50 mm. In some instances, the active area comprises a width of at least or about 0.5, 1, 1.5, 2, 2.5, 3, 5, 5, 10, 12, 14, 16, 18, 20, 25, 30, 35, 40, 45, 50, 60, 70, 80, or more than 80 mm. In some instances, the active area comprises a height of at least or about 0.5, 1, 1.5, 2, 2.5, 3, 5, 5, 10, 12, 14, 16, 18, 20, 25, 30, 35, 40, 45, 50, 60, 70, 80, or more than 80 mm.

In some instances, the solid support has a number of sites (e.g., spots) or positions for polynucleotides synthesis. In some instances, the solid support may be used to storage of polynucleotides. In some instances, the solid support comprises up to or about 10,000 by 10,000 positions in an area. In some instances, the solid support comprises between about 1000 and 20,000 by between about 1000 and 20,000 positions in an area. In some instances, the solid support comprises at least or about 10, 30, 50, 75, 100, 200, 300, 400, 500, 1000, 2000, 3000, 4000, 5000, 6000, 7000, 8000, 9000, 10,000, 12,000, 14,000, 16,000, 18,000, 20,000 positions by least or about 10, 30, 50, 75, 100, 200, 300, 400, 500, 1000, 2000, 3000, 4000, 5000, 6000, 7000, 8000, 9000, 10,000, 12,000, 14,000, 16.000, 18,000, 20,000 positions in an area. In some instances the area is up to 0.25, 0.5, 0.75, 1.0, 1.25, 1.5, or 2.0 inches squared. In some instances, the solid support comprises loci having a pitch of at least or about 0.1, 0.2, 0.25, 0.3, 0.4, 0.5, 1.0, 1.5, 2.0, 2.5, 3.0, 3.5, 4.0, 4.5, 5, 6, 7, 8, 9, 10, or more than 10 μm. In some instances, the solid support comprises loci having a pitch of about 5 μm. In some instances, the solid support comprises loci having a pitch of about 2 μm. In some instances, the solid support comprises loci having a pitch of about 1 μm. In some instances, the solid support comprises loci having a pitch of about 0.2 um. In some instances, the solid support comprises loci having a pitch of about 0.2 um to about 10 μm, about 0.2 to about 8 μm, about 0.5 to about 10 μm, about 1 μm to about 10 μm, about 2 μm to about 8 μm, about 3 μm to about 5 μm, about 1 μm to about 3 μm or about 0.5 um to about 3 μm. In some instances, the solid support comprises loci having a pitch of about 0.1 um to about 3 um.

In some instances, the solid support can be used for polynucleotide storage. In some instances, the solid support comprise a high capacity for storage of data. For example, the capacity of the solid support is at least or about 1, 2, 3, 4, 5, 6, 7, 8, 9, 10, 20, 50, 100, 200, 300, 400, 500, 600, 700, 800, 900, 1000, or more than 1000 petabytes. In some instances, the capacity of the solid support is between about 1 to about 10 petabytes or between about 1 to about 100 petabytes. In some instances, the capacity of the solid support is about 100 petabytes. In some examples, the polynucleotides are stored for a time on the solid support, and subsequently extracted from the solid support using the systems and methods provided herein. For example, polynucleotides on a solid support may be stored for days, months, years, or decades and subsequently extracted from the solid support using a flow cell, for recovery of information whole or in-part, or quality control of the polynucleotides. In some examples, one or more orientations of the flow cell has been optimized to maximize a liquid comprising the polynucleotides that can be recovered from the flow cell (e.g., angles α or β).

In some instances, the data is stored as arrays of packets as droplets. In some examples, the arrays of packets are addressable packets. In some examples, the packets are addressable using an electrode. In some instances, the data is stored as arrays of packets as droplets on a spot. In some instances, the data is stored as arrays of packets as dry wells. In some instances, the arrays comprise at least or about 1, 2, 3, 4, 5, 6, 7, 8, 9, 10, 20, 50, 100, 200, or more than 200 gigabytes of data. In some instances, the arrays comprise at least or about 1, 2, 3, 4, 5, 6, 7, 8, 9, 10, 20, 50, 100, 200, or more than 200 terabytes of data. In some instances, the arrays comprise at least or about 1, 2, 3, 4, 5, 6, 7, 8, 9, 10, 20, 50, 100, 200, or more than 200 petabytes of data. In some instances, the arrays comprise at least or about 1, 2, 3, 4, 5, 6, 7, 8, 9, 10, 20, 50, 100, 200, or more than 200 exabytes of data. In some instances, an item of information is stored in a background of data. For example, an item of information encodes for about 10 to about 100 terabytes of data and is stored in 1 petabyte of background data. In some instances, an item of information encodes for about 10 to about 100 petabytes of data and is stored in 1 terabyte of background data. In some instances, an item of information encodes for about 1 to about 100 exabytes of data and is stored in 1 petabyte of background data. In some instances, an item of information encodes for at least or about 1, 10, 20, 30, 40, 50, 60, 70, 80, 90, 100, 150, 200, 300, 400, 500, or more than 500 terabytes of data and is stored in 1, 10, 20, 30, 40, 50, 60, 70, 80, 90, 100, 150, 200, 300, 400, 500, or more than 500 petabytes of background data. In some instances, an item of information encodes for at least or about 1, 10, 20, 30, 40, 50, 60, 70, 80, 90, 100, 150, 200, 300, 400, 500, or more than 500 petabytes of data and is stored in 1, 10, 20, 30, 40, 50, 60, 70, 80, 90, 100, 150, 200, 300, 400, 500, or more than 500 exabytes of background data. In some instances, plates comprise a plurality of wells, such as 96, 384, 1024, or more wells.

Provided herein is a data storage system comprising a solid support, where following synthesis, the polynucleotides are collected in packets as one or more droplets. In some instances, the polynucleotides are collected in packets as one or more droplets and stored. In some instances, a number of droplets is at least or about 1, 10, 20, 50, 100, 200, 300, 500, 1000, 2500, 5000, 75000, 10,000, 25,000, 50,000, 75,000, 100,000, 1 million, 5 million, 10 million, 25 million, 50 million, 75 million, 100 million, 250 million, 500 million, 750 million, or more than 750 million droplets. In some instances, a droplet volume comprises 5, 10, 15, 20, 25, 30, 35, 40, 45, 50, 55, 60, 65, 70, 75, 80, 85, 90, 95, 100, or more than 100 um (micrometer) in diameter. In some instances, a droplet volume comprises 1-100 um, 10-90 um, 20-80 um, 30-70 um, or 40-50 um in diameter.

In some instances, the polynucleotides that are collectd in the packets comprise a similar sequence. In some instances, the polynucleotides further comprise a non-identical sequence to be used as a tag or barcode. For example, the non-identical sequence is used to index the polynucleotides stored on the solid support and to later search for specific polynucleotides based on the non-identical sequence. Exemplary tag or barcode lengths include barcode sequences comprising, without limitation, about 1, 2, 3, 4, 5, 6, 7, 8, 9, 10, 15, 20, 25 or more bases in length. In some instances, the tag or barcode comprise at least or about 10, 50, 75, 100, 200, 300, 400, or more than 400 base pairs in length.

Provided herein is a data storage system comprising a solid support, where the polynucleotides are collected in packets comprising redundancy. For example, the packets comprise about 100 to about 1000 copies of each polynucleotide. In some instances, the packets comprise at least or about 50, 75, 100, 200, 300, 400, 500, 600, 700, 800, 900, 1000, 1200, 1400, 1600, 1800, 2000, or more than 2000 copies of each polynucleotide. In some instances, the packets comprise about 1000× to about 5000× synthesis redundancy. Synthesis redundancy in some instances is at least or about 500×, 1000×, 1500×, 2000×, 2500×, 3000×, 3500×, 4000×, 5000×, 6000×, 7000×, 8000×, or more than 8000×. The polynucleotides that are synthesized using solid support based methods as described herein comprise various lengths. In some instances, the polynucleotides are synthesized and further stored on the solid support. In some instances, the polynucleotide length is in between about 100 to about 1000 bases. In some instances, the polynucleotides comprise at least or about 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, 2000, or more than 2000 bases in length.

### De Novo Polynucleotide Synthesis

Provided herein are systems and methods for synthesis of libraries of polynucleotides on a substrate. In some instances, the library comprising a plurality of polynucleotides from the encoding scheme are synthesized. In some examples, the library comprising the plurality of polynucleotides from the encoding scheme encode a pool of the plurality of pools. In some examples, the library comprising the plurality of polynucleotides from the encoding scheme encode an index pool. In some instances, methods comprise use of electrochemical deprotection. In some instances, the substrate is a flexible substrate. In some instances, at least 10¹⁰, 10¹¹, 10¹², 10¹³, 10¹⁴, or 10¹⁵ bases are synthesized in one day. In some instances, at least 10×10⁸, 10×10⁹, 10×10¹⁰, 10×10¹¹, or 10×10¹² polynucleotides are synthesized in one day. In some cases, each polynucleotide synthesized comprises at least 20, 50, 100, 200, 300, 400 or 500 nucleobases. In some cases, these bases are synthesized with a total average error rate of less than about 1 in 100; 200; 300; 400; 500; 1000; 2000; 5000; 10000; 15000; 20000 bases. In some instances, these error rates are for at least 50%, 60%, 70%, 80%, 90%, 95%, 98%, 99%, 99.5%, or more of the polynucleotides synthesized. In some instances, these at least 90% a, 95%, 98%, 99%, 99.5%, or more of the polynucleotides synthesized do not differ from a predetermined sequence for which they encode. In some instances, the error rate for synthesized polynucleotides on a substrate using the methods and systems described herein is less than about 1 in 200, less than about 1 in 1,000, less than about 1 in 2,000, less than about 1 in 3,000, or less than about 1 in 5,000. Individual types of error rates include mismatches, deletions, insertions, and/or substitutions for the polynucleotides synthesized on the substrate. The term “error rate” refers to a comparison of the collective amount of synthesized polynucleotide to an aggregate of predetermined polynucleotide sequences. In some instances, synthesized polynucleotides disclosed herein comprise a tether of 12 to 25 bases. In some instances, the tether comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more bases.

Described herein are methods, systems, devices, and compositions wherein chemical reactions used in polynucleotide synthesis are controlled using electrochemistry. Electrochemical reactions in some instances are controlled by any source of energy, such as light, heat, radiation, or electricity. For example, electrodes are used to control chemical reactions as all or a portion of discrete loci on a surface. Electrodes in some instances are charged by applying an electrical potential to the electrode to control one or more chemical steps in polynucleotide synthesis. In some instances, these electrodes are addressable. Any number of the chemical steps described herein is in some instances controlled with one or more electrodes. Electrochemical reactions may comprise oxidations, reductions, acid/base chemistry, or other reaction that is controlled by an electrode. In some instances, electrodes generate electrons or protons that are used as reagents for chemical transformations. Electrodes in some instances directly generate a reagent such as an acid. In some instances, an acid is a proton. Electrodes in some instances directly generate a reagent such as a base. Acids or bases are often used to cleave protecting groups, or influence the kinetics of various polynucleotide synthesis reactions, for example by adjusting the pH of a reaction solution. Electrochemically controlled polynucleotide synthesis reactions in some instances comprise redox-active metals or other redox-active organic materials. In some instances, metal or organic catalysts are employed with these electrochemical reactions. In some instances, acids are generated from oxidation of quinones.

Control of chemical reactions is not limited to the electrochemical generation of reagents; chemical reactivity may be influenced indirectly through biophysical changes to substrates or reagents through electric fields (or gradients) which are generated by electrodes. In some instances, substrates include but are not limited to polynucleotides. In some instances, electrical fields which repel or attract specific reagents or substrates towards or away from an electrode or surface are generated. Such fields in some instances are generated by application of an electrical potential to one or more electrodes. For example, negatively charged polynucleotides are repelled from negatively charged electrode surfaces. Such repulsions or attractions of polynucleotides or other reagents caused by local electric fields in some instances provides for movement of polynucleotides or other reagents in or out of region of the synthesis device or structure. In some instances, electrodes generate electric fields which repel polynucleotides away from a synthesis surface, structure, or device. In some instances, electrodes generate electric fields which attract polynucleotides towards a synthesis surface, structure, or device. In some instances, protons are repelled from a positively charged surface to limit contact of protons with substrates or portions thereof. In some instances, repulsion or attractive forces are used to allow or block entry of reagents or substrates to specific areas of the synthesis surface. In some instances, nucleoside monomers are prevented from contacting a polynucleotide chain by application of an electric field in the vicinity of one or both components. Such arrangements allow gating of specific reagents, which may obviate the need for protecting groups when the concentration or rate of contact between reagents and/or substrates is controlled. In some instances, unprotected nucleoside monomers are used for polynucleotide synthesis. Alternatively, application of the field in the vicinity of one or both components promotes contact of nucleoside monomers with a polynucleotide chain. Additionally, application of electric fields to a substrate can alter the substrates reactivity or conformation. In an exemplary application, electric fields generated by electrodes are used to prevent polynucleotides at adjacent loci from interacting. In some instances, the substrate is a polynucleotide, optionally attached to a surface. Application of an electric field in some instances alters the three-dimensional structure of a polynucleotide. Such alterations comprise folding or unfolding of various structures, such as helices, hairpins, loops, or other 3-dimensional nucleic acid structure. Such alterations are useful for manipulating polynucleotides inside of wells, channels, or other structures. In some instances, electric fields are applied to a nucleic acid substrate to prevent secondary structures. In some instances, electric fields obviate the need for linkers or attachment to a solid support during polynucleotide synthesis.

A suitable method for polynucleotide synthesis on a substrate of this disclosure is a phosphoramidite-based synthesis of DNA. In some cases, a reagent for the phosphoramidite-based synthesis comprises any one of or a combination of a nucleoside phosphoramidite, an oxidizer, an activator, or a deblocker or the solvent comprises acetonitrile. In some instances, the phosphoramidite-based synthesis method comprises the controlled addition of a phosphoramidite building block. i.e. nucleoside phosphoramidite, to a growing polynucleotide chain in a coupling step that forms a phosphite triester linkage between the phosphoramidite building block and a nucleoside bound to the substrate. In some instances, the nucleoside phosphoramidite is provided to the substrate activated. In some instances, the nucleoside phosphoramidite is provided to the substrate with an activator. In some instances, nucleoside phosphoramidites are provided to the substrate in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition and linkage of a nucleoside phosphoramidite in the coupling step, the substrate is optionally washed. In some instances, the coupling step is repeated one or more additional times, optionally with a wash step between nucleoside phosphoramidite additions to the substrate. In some instances, a polynucleotide synthesis method used herein comprises 1, 2, 3 or more sequential coupling steps. Prior to coupling, in many cases, the nucleoside bound to the substrate is de-protected by removal of a protecting group, where the protecting group functions to prevent polymerization. Protecting groups may comprise any chemical group that prevents extension of the polynucleotide chain. In some instances, the protecting group is cleaved (or removed) in the presence of an acid. In some instances, the protecting group is cleaved in the presence of a base. In some instances, the protecting group is removed with electromagnetic radiation such as light, heat, or other energy source. In some instances, the protecting group is removed through an oxidation or reduction reaction. In some instances, a protecting group comprises a triarylmethyl group. In some instances, a protecting group comprises an aryl ether. In some instances, a protecting comprises a disulfide. In some instances a protecting group comprises an acid-labile silane. In some instances, a protecting group comprises an acetal. In some instances, a protecting group comprises a ketal. In some instances, a protecting group comprises an enol ether. In some instances, a protecting group comprises a methoxybenzyl group. In some instances, a protecting group comprises an azide. In some instances, a protecting group is 4,4′-dimethoxytrityl (DMT). In some instances, a protecting group is a tert-butyl carbonate. In some instances, a protecting group is a tert-butyl ester. In some instances, a protecting group comprises a base-labile group.

Following coupling, phosphoramidite polynucleotide synthesis methods optionally comprise a capping step. In a capping step, the growing polynucleotide is treated with a capping agent. A capping step generally serves to block unreacted substrate-bound 5′—OH groups after coupling from further chain elongation, preventing the formation of polynucleotides with internal base deletions. Further, phosphoramidites activated with 1H-tetrazole often react, to a small extent, with the O6 position of guanosine. Without being bound by theory, upon oxidation with I2/water, this side product, possibly via O6-N7 migration, undergoes depurination. The apurinic sites can end up being cleaved in the course of the final deprotection of the polynucleotide thus reducing the yield of the full-length product. The O6 modifications may be removed by treatment with the capping reagent prior to oxidation with I2/water. In some instances, inclusion of a capping step during polynucleotide synthesis decreases the error rate as compared to synthesis without capping. As an example, the capping step comprises treating the substrate-bound polynucleotide with a mixture of acetic anhydride and 1-methylimidazole. Following a capping step, the substrate is optionally washed.

Following addition of a nucleoside phosphoramidite, and optionally after capping and one or more wash steps, a substrate described herein comprises a bound growing nucleic acid that may be oxidized. The oxidation step comprises oxidizing the phosphite triester into a tetracoordinated phosphate triester, a protected precursor of the naturally occurring phosphate diester internucleoside linkage. In some instances, phosphite triesters are oxidized electrochemically. In some instances, oxidation of the growing polynucleotide is achieved by treatment with iodine and water, optionally in the presence of a weak base such as a pyridine, lutidine, or collidine. Oxidation is sometimes carried out under anhydrous conditions using tert-Butyl hydroperoxide or (1S)-(+)-(10-camphorsulfonyl)-oxaziridine (CSO). In some methods, a capping step is performed following oxidation. A second capping step allows for substrate drying, as residual water from oxidation that may persist can inhibit subsequent coupling. Following oxidation, the substrate and growing polynucleotide is optionally washed. In some instances, the step of oxidation is substituted with a sulfurization step to obtain polynucleotide phosphorothioates, wherein any capping steps can be performed after the sulfurization. Many reagents are capable of the efficient sulfur transfer, including, but not limited to, 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

For a subsequent cycle of nucleoside incorporation to occur through coupling, a protected 5′ end (or 3′ end, if synthesis is conducted in a 5′ to 3′ direction) of the substrate bound growing polynucleotide is be removed so that the primary hydroxyl group can react with a next nucleoside phosphoramidite. In some instances, the protecting group is DMT and deblocking occurs with trichloroacetic acid in dichloromethane. In some instances, the protecting group is DMT and deblocking occurs with electrochemically generated protons. Conducting detritylation for an extended time or with stronger than recommended solutions of acids may lead to increased depurination of solid support-bound polynucleotide and thus reduces the yield of the desired full-length product. Methods and compositions described herein provide for controlled deblocking conditions limiting undesired depurination reactions. In some instances, the substrate bound polynucleotide is washed after deblocking. In some cases, efficient washing after deblocking contributes to synthesized polynucleotides having a low error rate.

Methods for the synthesis of polynucleotides on a substrate described herein may involve an iterating sequence of the following steps: application of a protected monomer to a surface of a substrate feature to link with either the surface, a linker or with a previously deprotected monomer; deprotection of the applied monomer so that it can react with a subsequently applied protected monomer; and application of another protected monomer for linking. One or more intermediate steps include oxidation and/or sulfurization. In some instances, one or more wash steps precede or follow one or all of the steps.

Methods for the synthesis of polynucleotides on a substrate described herein may comprise an oxidation step. For example, methods involve an iterating sequence of the following steps: application of a protected monomer to a surface of a substrate feature to link with either the surface, a linker or with a previously deprotected monomer; deprotection of the applied monomer so that it can react with a subsequently applied protected monomer; application of another protected monomer for linking, and oxidation and/or sulfurization. In some instances, one or more wash steps precede or follow one or all of the steps.

Methods for the synthesis of polynucleotides on a substrate described herein may further comprise an iterating sequence of the following steps: application of a protected monomer to a surface of a substrate feature to link with either the surface, a linker or with a previously deprotected monomer; deprotection of the applied monomer so that it can react with a subsequently applied protected monomer; and oxidation and/or sulfurization. In some instances, one or more wash steps precede or follow one or all of the steps.

Methods for the synthesis of polynucleotides on a substrate described herein may further comprise an iterating sequence of the following steps: application of a protected monomer to a surface of a substrate feature to link with either the surface, a linker or with a previously deprotected monomer, and oxidation and/or sulfurization. In some instances, one or more wash steps precede or follow one or all of the steps.

Methods for the synthesis of polynucleotides on a substrate described herein may further comprise an iterating sequence of the following steps: application of a protected monomer to a surface of a substrate feature to link with either the surface, a linker or with a previously deprotected monomer, deprotection of the applied monomer so that it can react with a subsequently applied protected monomer; and oxidation and/or sulfurization. In some instances, one or more wash steps precede or follow one or all of the steps.

In some instances, polynucleotides are synthesized with photolabile protecting groups, where the hydroxyl groups generated on the surface are blocked by photolabile-protecting groups. When the surface is exposed to UV light, such as through a photolithographic mask, a pattern of free hydroxyl groups on the surface may be generated. These hydroxyl groups can react with photoprotected nucleoside phosphoramidites, according to phosphoramidite chemistry. A second photolithographic mask can be applied and the surface can be exposed to UV light to generate second pattern of hydroxyl groups, followed by coupling with 5′-photoprotected nucleoside phosphoramidite. Likewise, patterns can be generated and oligomer chains can be extended. Without being bound by theory, the lability of a photocleavable group depends on the wavelength and polarity of a solvent employed and the rate of photocleavage may be affected by the duration of exposure and the intensity of light. This method can leverage a number of factors such as accuracy in alignment of the masks, efficiency of removal of photo-protecting groups, and the yields of the phosphoramidite coupling step. Further, unintended leakage of light into neighboring sites can be minimized. The density of synthesized oligomer per spot can be monitored by adjusting loading of the leader nucleoside on the surface of synthesis.

The surface of a substrate described herein that provides support for polynucleotide synthesis may be chemically modified to allow for the synthesized polynucleotide chain to be cleaved from the surface. In some instances, the polynucleotide chain is cleaved at the same time as the polynucleotide is deprotected. In some cases, the polynucleotide chain is cleaved after the polynucleotide is deprotected. In an exemplary scheme, a trialkoxysilyl amine such as (CH₃CH₂O)₃Si—(CH₂)₂—NH₂ is reacted with surface SiOH groups of a substrate, followed by reaction with succinic anhydride with the amine to create an amide linkage and a free OH on which the nucleic acid chain growth is supported. Cleavage includes gas cleavage with ammonia or methylamine. In some instances cleavage includes linker cleavage with electrically generated reagents such as acids or bases. In some instances, once released from the surface, polynucleotides are assembled into larger polynucleotides that are sequenced and decoded to extract stored information.

The surfaces described herein can be reused after polynucleotide cleavage to support additional cycles of polynucleotide synthesis. For example, the linker can be reused without additional treatment/chemical modifications. In some instances, a linker is non-covalently bound to a substrate surface or a polynucleotide. In some embodiments, the linker remains attached to the polynucleotide after cleavage from the surface. Linkers in some embodiments comprise reversible covalent bonds such as esters, amides, ketals, beta substituted ketones, heterocycles, or other group that is capable of being reversibly cleaved. Such reversible cleavage reactions are in some instances controlled through the addition or removal of reagents, or by electrochemical processes controlled by electrodes. Optionally, chemical linkers or surface-bound chemical groups are regenerated after a number of cycles, to restore reactivity and remove unwanted side product formation on such linkers or surface-bound chemical groups.

Alternatively, the polymer synthesis can be enzymatic DNA synthesis. In some cases, the enzymatic DNA synthesis uses water as a solvent and the reagent is an enzyme terminal deoxynucleotidyl transferase (TdT) or a deblocker. In some cases, enzymatic synthesis of DNA uses a template-independent DNA polymerase, terminal deoxynucleotidyl transferase (TdT), which is a protein that evolved to rapidly catalyze the linkage of naturally occurring dNTPs. TdT adds nucleotides indiscriminately so it is stopped from continuing unregulated synthesis by various techniques such a tethering the TdT, creating variant enzymes, and using nucleotides that include reversible terminators to prevent chain elongation. TdT activity is maximized at approximately 37° C. and performs enzymatic reactions in an aqueous environment.

### Extraction

In some instances, the polynucleotides are deprotected, cleaved, and/or eluted from the synthesizer unit 1810 and transferred to another module in the system. In some instances, the polynucleotides are transferred from the synthesizer unit 1810 on the solid support. In some instances, a robotic system 1830 or fluidic tube is used to transport the polynucleotides to another module in the system. A robotic system 1830 may be controlled by a controller 1835. A robotic system generally comprises a system for manipulation of a plurality of polynucleotides. In some instances, the robotic system is used to manipulate a structure comprising a plurality of polynucleotides, such as those described herein. Manipulation can comprise, by way of non-limiting example, moving, storing, retrieving, handling, transferring, or any combination thereof. The robotic system may be similar to those used in semiconductor processing to move trays of wafers and chips between processing devices. A robotic system 1830 may be used to select and transfer polynucleotides between modules of the system. For example, a robotic system 1835 may include a tag reader to verify a structure in a storage unit 1815. In some instances, the robotic system 1835 comprises a reader of a tag (e.g., RFID reader, barcode reader, etc.) and the structure in the storage unit 1815 comprises a tag (e.g., RFID tag, barcode, etc.). Once verified, the robotic system 1830 may transfer the structure to a component of the system. Additionally, the robotic system 1830 may transfer the structure to a precise location in a component of the system. In some instances, the robotic system can allow for polynucleotides to be added and/or removed from modules in the data storage system. In some instances, the robotic system allows for a structure comprising a plurality of polynucleotides to be placed and/or retrieved from a location in an identifiable layout in the storage unit 1815. The robotic system 1830 may be controlled using a controller 1835 as further described herein.

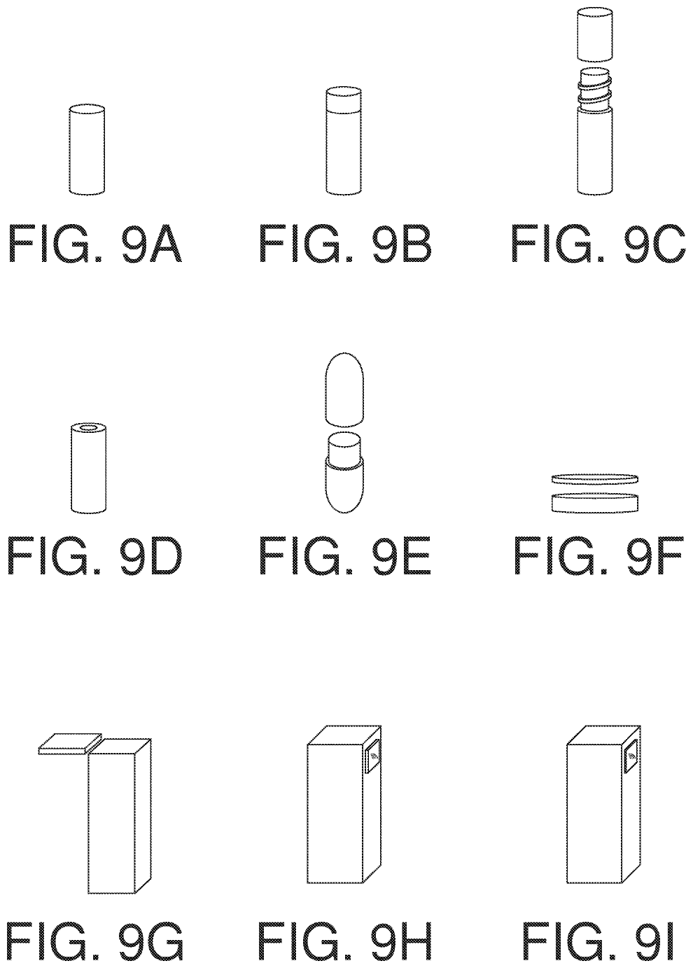

In some instances, one or more droplets comprising polynucleotides are transferred from a synthesizer unit 1810 to a storage unit 1815. In some instances, some or all of the polynucleotides synthesized on a solid support are transferred to a structure for storage. The structure may have a variety of shapes and sizes, such as those described herein (e.g., FIGS. 9A-9I). The structure may further comprise a tag, such as those described herein (e.g., FIGS. 9H-9I). The tag can comprise an RFID tag. In some examples, the synthesizer unit 1810 is connected to or is coextensive with a system or platform for biomolecule extraction or storage. Thus in some instances, the polynucleotides are extracted using a system or platform comprising a flow cell, oriented to maximize the recovery of fluid comprising the polynucleotides. The extracted polynucleotides can be collected in a structure for subsequent storage. In some instances, an intermediate storage chamber is used to collect polynucleotides from a specific flow cell (or a plurality of flow cells) or flow cell block (or a plurality of flow cell blocks). In some instances, polynucleotides are transferred from an intermediate storage chamber to another storage chamber.

The system can further comprise one or more mechanisms for transferring a substrate to and from the system as part of a larger platform for generally for biomolecule storage. Such a platform can generally comprise one or more components, such as a flow cell comprising a cavity for holding a substrate comprising biomolecules, a reservoir, and a pump, and any other suitable components of the system described herein. The platform can further comprise an apparatus comprising at least one logic element for performing one or more operations in the platform. In some instances, one or more operations are performed based on sensor data from one or more components of the biomolecule extraction system. In some instances, the at least one logic element comprises a programmable logic controller (PLC), programable logic array (PLA), programmable array logic (PAL), generic logic array (GLA), complex programmable logic decide (CPLD), field programable gate array (FPGA), or application-specific integrated circuit (ASIC). Such an apparatus may be in communication with or coextensive with a controller of a system for biomolecule extraction, as described herein.

An apparatus in a platform may perform one or more operations, such as (i) determining a timing for opening or closing one or more valves connecting the one or more components, (ii) adjusting one or more parameters of the biomolecule extraction system, wherein the one or more parameters comprises a fill rate of the liquid, a volume of the liquid, the liquid, an evacuation rate of the liquid, a capillary number, one or more angles of the flow cell relative to a horizontal plane, or any combination thereof, (iii) determining a recovery efficiency of the liquid evacuated from the cavity comprising biomolecules, or (iv) any combination thereof. In some instances, one or more metrics, parameters, sensor data, or any combination thereof, may be displayed in real time to a user interface. In some instances, one or more metrics, parameters, sensor data, or any combination thereof, may be analyzed in real time to detect anomalies in a system. In some instances, an anomaly results in a system fault. In some instances, a controller executes one or more actions in response to a system fault. In some instances actions include one or more of logging the fault, ejecting the contents of the flow cell or flow cell block into waste, resetting a flow cell or flow cell block, and marking a flow cell or flow cell block as faulty (e.g., preventing further synthesis at this device). In some instances, one or more metrics, parameters, sensor data, or any combination thereof, may be stored on the cloud, random access memory, hard-disk drive, solid-state drive, flash memory device, or any variation thereof. In some examples, the apparatus is communicably coupled to a cloud computing resource, which can be used to execute any of the operations provided herein. In some instances, a system provided herein comprises a graceful fail-over system.

In some instances, the platform comprises a mechanism for transferring the substrate from the flow cell to another processing device, such as a substrate reaction device (e.g., for incubating a substrate with a reactant under reaction conditions, such as a synthesis module, a sequencing module, or an amplification module), a washing device, a scanning device, or any combinations thereof. Such mechanism can also be provided to move the substrate from a printing station (e.g., inkjet printing) to the cavity of the flow cell. Transfer mechanisms can include, but are not limited to, robotic arms, and the like, which can be controlled by an apparatus and/or controllers, as described herein. In some instances, a transfer robot is mounted on a platform of an apparatus used in for synthesis. The transfer robot may include a base, an arm that is movably mounted on the base, and a grasping element adapted to grasp the substrate during transport that is attached to the arm. The element for grasping the substrate may be, for example, movable finger-like projections, and the like. In one aspect, in use, the robotic arm is activated so that the substrate is grasped by the grasping element. The arm of the robot is moved so that the substrate is delivered to the flow cell from a printing device. Other componentry may be used to position the substrate, e.g., motors, pistons, conveyers, cranks, levers, etc., where such will be obvious to those of skill in the art in view of the disclosure. As noted above, in some instances, a substrate may be positioned on a substrate holder or lift mechanism within the chamber of the flow cell. In some instances, the holder may be adapted to be moveable to position the substrate appropriately.

The methods for biomolecule extraction may employ a device, system, platform, or any combination thereof, provided herein. In some instances, the methods for biomolecule extraction employ a flow cell described herein. In some instances, methods are developed to optimize one or more conditions or parameters of a device, system, or platform comprising a flow cell, in order to maximize the fluid recovered from a cavity of the flow cell. In some examples, methods may be developed to maximum extraction of liquid from a cavity by changing a process parameter, such as angles α and β as defined herein, or a flow rate of evacuation (e.g., air flow rate for displacing fluid from the cavity of the flow cell). In some examples, methods may be developed to demonstrate a relationship between volume of liquid recovered to the capillary number for the liquid (e.g., water, extraction reagent, etc.). In some examples, methods may be developed in order to apply them to a reagent used for biomolecule extraction, such as oligomer extraction, from a surface. The system can comprise a flow cell comprising the cavity. In some examples, the cavity comprises a planar surface that is substantially parallel to a body force, such as gravity. In some examples, the flow cell comprises a first opening and a second opening, which can be used to fill or evacuate liquid from the cavity. In some examples, the flow cell comprises only two openings, which can be used to fill or evacuate liquid from the cavity. In some examples, the flow cell comprises a plurality of openings, which can be used to fill or evacuate liquid from the cavity. The system can further comprise a reservoir for holding a liquid, which can be connected to the first opening using any suitable material known in the art (e.g., PFA). The system can further comprise a pump, which can be connected to the second opening using any suitable material known in the art (e.g., PFA).

The method can further comprise introducing the liquid to the cavity of the flow cell. For example, the liquid may be the liquid housed in the reservoir, which can be introduced to the cavity via a suitably conduit. In some instances, cavity is filled at a rate (fill rate) of about 0 to 50 uL/s. In some instances, the fill rate is about 0 to 0.5, 0 to 1, 0 to 2.5, 0 to 5, 0 to 10, 0 to 15, 0 to 20, 0 to 25, 0 to 30, 0 to 50, 0.5 to 1, 0.5 to 2.5, 0.5 to 5, 0.5 to 10, 0.5 to 15, 0.5 to 20, 0.5 to 25, 0.5 to 30, 0.5 to 50, 1 to 2.5, 1 to 5, 1 to 10, 1 to 15, 1 to 20, 1 to 25, 1 to 30, 1 to 50, 2.5 to 5, 2.5 to 10, 2.5 to 15, 2.5 to 20, 2.5 to 25, 2.5 to 30, 2.5 to 50, 5 to 10, 5 to 15, 5 to 20, 5 to 25, 5 to 30, 5 to 50, 10 to 15, 10 to 20, 10 to 25, 10 to 30, 10 to 50, 15 to 20, 15 to 25, 15 to 30, 15 to 50, 20 to 25, 20 to 30, 20 to 50, 25 to 30, 25 to 50, or 30 to 50 uL/s. In some instances, the fill rate is about 0, 0.5, 1, 2.5, 5, 10, 15, 20, 25, 30, or 50 uL/s. In some instances, the fill rate is at least about 0, 0.5, 1, 2.5, 5, 10, 15, 20, 25, or 30 uL/s. In some instances, the fill rate is at most about 0.5, 1, 2.5, 5, 10, 15, 20, 25, 30, or 50 uL/s. In some instances, the volume of the liquid filling the cavity is about 0 to 1000 mL. In some instances, the volume is about 0 to 100, 0 to 200, 0 to 300, 0 to 400, 0 to 500, 0 to 600, 0 to 700, 0 to 800, 0 to 900, 0 to 1,000, 100 to 200, 100 to 300, 100 to 400, 100 to 500, 100 to 600, 100 to 700, 100 to 800, 100 to 900, 100 to 1,000, 200 to 300, 200 to 400, 200 to 500, 200 to 600, 200 to 700, 200 to 800, 200 to 900, 200 to 1,000, 300 to 400, 300 to 500, 300 to 600, 300 to 700, 300 to 800, 300 to 900, 300 to 1000, 400 to 500, 400 to 600, 400 to 700, 400 to 800, 400 to 900, 400 to 1000, 500 to 600, 500 to 700, 500 to 800, 500 to 900, 500 to 1000, 600 to 700, 600 to 800, 600 to 900, 600 to 1000, 700 to 800, 700 to 900, 700 to 1000, 800 to 900, 800 to 1000, or 900 to 1000 mL. In some instances, the volume is about 0, 100, 200, 300, 400, 500, 600, 700, 800, 900, or 1000 mL. In some instances, the volume is at least about 0, 100, 200, 300, 400, 500, 600, 700, 800, or 900 mL. In some instances, the volume is at most about 100, 200, 300, 400, 500, 600, 700, 800, 900, or 1000 mL.

The method can further comprise evacuating the liquid to the cavity of the flow cell. For example, the liquid can be evacuated from the cavity using a pump, which can be used to introduce air into the cavity via a suitable conduit, and evacuate liquid from the cavity. In some instances, cavity is evacuated at a rate (evacuation rate) of about 0 to 50 uL/s. In some instances, the evacuation rate is about 0 to 0.5, 0 to 1.0 to 2.0 to 5.0 to 7.0 to 10.0 to 15.0 to 20.0 to 25.0 to 30.0 to 50, 0.5 to 1, 0.5 to 2, 0.5 to 5, 0.5 to 7, 0.5 to 10, 0.5 to 15, 0.5 to 20, 0.5 to 25, 0.5 to 30, 0.5 to 50, 1 to 2, 1 to 5, 1 to 7, 1 to 10, 1 to 15, 1 to 20, 1 to 25, 1 to 30, 1 to 50, 2 to 5, 2 to 7, 2 to 10, 2 to 15, 2 to 20, 2 to 25, 2 to 30, 2 to 50, 5 to 7, 5 to 10, 5 to 15, 5 to 20, 5 to 25, 5 to 30, 5 to 50, 7 to 10, 7 to 15, 7 to 20, 7 to 25, 7 to 30, 7 to 50, 10 to 15, 10 to 20, 10 to 25, 10 to 30, 10 to 50, 15 to 20, 15 to 25, 15 to 30, 15 to 50, 20 to 25, 20 to 30, 20 to 50, 25 to 30, 25 to 50, or 30 to 50 uL/s. In some instances, the evacuation rate is about 0, 0.5, 1, 2, 5, 7, 10, 15, 20, 25, 30, or 50 uL/s. In some instances, the evacuation rate is at least about 0, 0.5, 1, 2, 5, 7, 10, 15, 20, 25, or 30 uL/s. In some instances, the evacuation rate is at most about 0.5, 1, 2, 5, 7, 10, 15, 20, 25, 30, or 50 uL/s.

The method can further comprise determining an amount of the liquid recovered from the cavity. In some instances, determining the amount of the liquid recovered from the cavity comprises comparing a property of the liquid after evacuating the liquid from the cavity to the property of the liquid before filling the cavity with the liquid. In some examples, the property comprises weight, density, or volume. Thus in some examples, determining the amount of the liquid recovered from the cavity comprises comparing a weight of the liquid in a reservoir after evacuating the liquid from the cavity to the property of the liquid before filling the cavity with the liquid. In some instances, determining the amount of the liquid recovered from the cavity further comprises determining a recovery efficiency. The recovery efficiency may be determined by the percentage, fraction, or ratio of the amount of liquid evacuated and recovered from the cavity to the amount of liquid that filled the cavity. In some instances, one or more sensors are configured to measure the extent of extraction. In some instances, a recovery efficiency may be measured using concentration. As an example, the concentration of biomolecules initially present within the cavity and/or the concentration retrieved can be measured by spectrophotometric means (e.g., A260/A280 readings), fluorescence, secondary reactions, or any other suitable method known in the art.

A method for determining one or more conditions for recovering a maximum amount of liquid from a cavity in a flow cell can further comprise adjusting a parameter of the system. In some instances, the parameter comprises a fill rate of the liquid, a volume of the liquid, the liquid, an evacuation rate of the liquid, a capillary number, one or more angles of the flow cell relative to a horizontal plane, or any combination thereof. In some examples, the one or more angles comprises an angle relative to a horizontal x-y plane. In some examples, the horizontal x-y plane is perpendicular to the body force, such as gravity. In some instances, the one or more angles of the flow cell is angle α, β, or both, as defined herein. In some examples, adjusting one or more angles of the flow cell comprises adjusting angle α, where α can be adjusted between 0° to 90°. In some examples, adjusting one or more angles of the flow cell comprises adjusting angle β, where β can be adjusted between 0° to 45°. In some examples, the fill rate is adjusted between 0 to 50 uL/s. In some examples, the evacuation rate is adjusted between 0 to 50 uL/s. In some examples, the volume of the liquid is adjusted between 0 mL to 1000 mL. In some examples, the capitally number is adjusted to a value from 0 to 1 (e.g., 0, 0.1, 0.2, 0.3, 0.4, 0.5, 0.6, 0.7, 0.8, 0.9, 1, etc.). Once a parameter or condition is adjusted, one or more operations in the method provided herein may be repeated to determine the one or more conditions for recovering the maximum amount of liquid from the cavity of the flow cell.

Further provided herein are methods for extracting biomolecules. In some instances, a flow cell, system, or platform is deployed once one or more conditions for maximizing the liquid from a cavity of a flow cell is determined. Such a method can comprise providing a system, comprising a flow cell with a cavity, a reservoir for holding liquid, and a pump. In some examples, the flow cell is oriented such that a planar surface of the cavity is substantially parallel to a body force, such as gravity. In some examples, the flow cell is oriented such that α>0°, β>0°, or both. In some examples, the flow cell is oriented such that α≤90°, β≤45° or both. In some examples, the flow cell is oriented such that 0°<α≤90°, 0°<β≤45° or both.

Methods for extracting biomolecules can comprise providing a substrate. In some instances, a substrate comprises a plurality of biomolecules. In some instances, extracting biomolecules comprises cleave biomolecules and transferring the biomolecules to a chamber. In some instances, the substrate comprises one or more chemicals or molecules. The molecules can comprise, but are not limited to, polymers (e.g., peptides, proteins, nucleic acids or mimetics thereof, e.g. peptide nucleic acids, LNA molecules, UNA molecules), polysaccharides, phospholipids, or any combination thereof, where the polymers may be hetero- or homopolymeric. In some examples, the substrate comprises cells or tissue sections stably immobilized thereto. In some instances, the substrate is placed in the cavity of the flow cell manually. In some instances, the substrate is placed in the cavity of the flow cell using an automated system. The automated system may comprise, but is not limited to, a transfer mechanism (e.g., robotic system, such as a robotic arm), described herein.

Methods can further comprise exposing the substrate in the cavity of a flow cell to a fluid. In some instances, the method comprises introducing the liquid to the cavity of the flow cell. In some examples, the liquid is introduced through a first opening of a flow cell or a first plurality of openings of a flow cell. As an example, a fluid may be filled through a single hole located at the bottom of a flow cell, thus introducing the liquid to the cavity of the flow cell and exposing the substrate in the cavity to the liquid. In some examples, the method comprises removing liquid from the cavity, such as removing the liquid in excess from the second opening after introducing the liquid to the cavity of the flow cell. In some instances, liquid is removed from the cavity through a single hole located at the top of the flow cell. In some instances, liquid is introduced to, or liquid is removed from the cavity through a plurality of holes. In some instances, cavity is filled at a rate (fill rate) of about 0 to 50 uL/s, as described herein. In some instances, the volume of the liquid filling the cavity is about 0 to 1000 mL, as described herein. In some examples, a cavity may be filled by a first fluid, followed by a second fluid, thus exposing the substrate to the first fluid, followed by the second fluid. In some examples, the fluid may be used to wash the substrate. In some examples, the fluid comprises a reagent that can react with a biomolecule on the substrate. In some examples, the biomolecule comprise nucleic acid molecules or polynucleotides. In some examples, the fluid comprises fluid for coupling, capping, oxidizing, sulfurizing, deblocking, or washing the substrate or biomolecules on the substrate. In some examples, the fluid is used to remove or cleave biomolecules on a surface of the substrate. The fluid may comprise, by way of non-limiting example, water, acetone, acetonitrile, methanol, ethanol, isopropyl alcohol, or tert-butylamine. In some examples, the fluid comprises tert-butylamine, water, and methanol, for example at a ratio of about 1:1:1, 1:1:2, 1:2:1.2:1:1, 1:2:2.2:1:2, or 2:2:1.

The fluid may comprise one or more properties suitable for use in the flow cell device or systems described herein. The one or more properties may comprise, by way of non-limiting example, surface tension, viscosity, density, vapor pressure, capillarity, cavitation, specific weight, specific volume, specific gravity, temperature, or pressure. In some examples, the fluid has a surface tension of about 0.001 to 0.1 N/m. In some examples, the fluid has a surface tension of about 0.001 to 0.002, 0.001 to 0.005, 0.001 to 0.01, 0.001 to 0.02, 0.001 to 0.03, 0.001 to 0.04, 0.001 to 0.05, 0.001 to 0.06, 0.001 to 0.07, 0.001 to 0.08, 0.001 to 0.09, 0.001 to 0.1, 0.002 to 0.005, 0.002 to 0.01, 0.002 to 0.02, 0.002 to 0.03, 0.002 to 0.04, 0.002 to 0.05, 0.002 to 0.06, 0.002 to 0.07, 0.002 to 0.08, 0.002 to 0.09, 0.002 to 0.1, 0.005 to 0.01, 0.005 to 0.02, 0.005 to 0.03, 0.005 to 0.04, 0.005 to 0.05, 0.005 to 0.06, 0.005 to 0.07, 0.005 to 0.08, 0.005 to 0.09, 0.005 to 0.1, 0.01 to 0.02, 0.01 to 0.03, 0.01 to 0.04, 0.01 to 0.05, 0.01 to 0.06, 0.01 to 0.07, 0.01 to 0.08, 0.01 to 0.09, 0.01 to 0.1, 0.02 to 0.03, 0.02 to 0.04, 0.02 to 0.05, 0.02 to 0.06, 0.02 to 0.07, 0.02 to 0.08, 0.02 to 0.09, 0.02 to 0.1, 0.03 to 0.04, 0.03 to 0.05, 0.03 to 0.06, 0.03 to 0.07, 0.03 to 0.08, 0.03 to 0.09, 0.03 to 0.1, 0.04 to 0.05, 0.04 to 0.06, 0.04 to 0.07, 0.04 to 0.08, 0.04 to 0.09, 0.04 to 0.1, 0.05 to 0.06, 0.05 to 0.07, 0.05 to 0.08, 0.05 to 0.09, 0.05 to 0.1, 0.06 to 0.07, 0.06 to 0.08, 0.06 to 0.09, 0.06 to 0.1, 0.07 to 0.08, 0.07 to 0.09, 0.07 to 0.1, 0.08 to 0.09, 0.08 to 0.1, or 0.09 to 0.1 N/m. In some examples, the fluid has a surface tension of about 0.001, 0.002, 0.005, 0.01, 0.02, 0.03, 0.04, 0.05, 0.06, 0.07, 0.08, 0.09, or 0.1 N/m. In some examples, the fluid has a surface tension of at least about 0.001, 0.002, 0.005, 0.01, 0.02, 0.03, 0.04, 0.05, 0.06, 0.07, 0.08, or 0.09 N/m. In some examples, the fluid has a surface tension of at most about 0.002, 0.005, 0.01, 0.02, 0.03, 0.04, 0.05, 0.06, 0.07, 0.08, 0.09, or 0.1 N/m. In some examples, the fluid has a viscosity of about 1×10³ to 5×10³ Pa s. In some examples, the fluid has a viscosity of 0.5×10⁻³ to 1×10⁻³, 0.5×10⁻³ to 1.5×10⁻³, 0.5×10⁻³ to 2×10⁻³, 0.5×10⁻³ to 2.5×10⁻³, 0.5×10⁻³ to 3×10⁻³, 0.5×10⁻³ to 3.5×10⁻³, 0.5×10⁻³ to 4×10⁻³, 0.5×10⁻³ to 4.5×10⁻³, 0.5×10⁻³ to 5×10⁻³, 1×10⁻³ to 1.5×10⁻³, 1×10⁻³ to 2×10⁻³, 1×10⁻³ to 2.5×10⁻³, 1×10⁻³ to 3, 1×10⁻³ to 3.5×10⁻³, 1×10⁻³ to 4×10⁻³, 1×10⁻³ to 4.5×10⁻³, 1×10⁻³ to 5×10⁻³, 1.5×10⁻³ to 2×10⁻³, 1.5×10⁻³ to 2.5×10⁻³, 1.5×10⁻³ to 3×10⁻³, 1.5×10⁻³ to 3.5×10⁻³, 1.5×10⁻³ to 4×10⁻³, 1.5×10⁻³ to 4.5×10⁻³, 1.5×10⁻³ to 5×10⁻³, 2×10⁻³ to 2.5×10⁻³, 2×10⁻³ to 3×10⁻³, 2×10⁻³ to 3.5×10⁻³, 2×10⁻³ to 4×10⁻³, 2×10⁻³ to 4.5×10⁻³, 2×10⁻³ to 5×10⁻³, 2.5×10⁻³ to 3×10⁻³, 2.5×10⁻³ to 3.5×10⁻³, 2.5×10⁻³ to 4×10⁻³, 2.5×10⁻³ to 4.5×10⁻³, 2.5×10⁻³ to 5×10⁻³, 3×10⁻³ to 3.5×10⁻³, 3×10⁻³ to 4×10⁻³, 3×10⁻³ to 4.5×10⁻³, 3×10⁻³ to 5×10⁻³, 3.5×10⁻³ to 4×10⁻³, 3.5×10 to 4.5×10⁻³, 3.5×10⁻³ to 5×10⁻³, 4×10⁻³ to 4.5×10⁻³, 4×10⁻³ to 5×10⁻³, or 4.5×10⁻³ to 5×10⁻³ Pa s. In some examples, the fluid has a viscosity of about 0.5×10⁻³, 1×10, 1.5×10⁻³, 2×10⁻³, 2.5×10⁻³, 3×10⁻³, 3.5×10⁻³, 4×10⁻³, 4.5×10⁻³, or 5×10⁻³ Pa s. In some examples, the fluid has a viscosity of about 0.5×10⁻³, 1×10⁻³, 1.5×10⁻³, 2×10⁻³, 2.5×10⁻³, 3×10⁻³, 3.5×10⁻³, 4×10⁻³, 4.5×10⁻³, or 5×10⁻³ Pa s. In some examples, the fluid has a viscosity of at least about 0.5×10⁻³, 1×10⁻³, 1.5×10⁻³, 2×10⁻³, 2.5×10⁻³, 3×10⁻³, 3.5×10⁻³, 4×10⁻³, or 4.5×10⁻³ Pa s. In some examples, the fluid has a viscosity of at most about 1×10⁻³, 1.5×10⁻³, 2×10⁻³, 2.5×10⁻³, 3×10⁻³, 3.5×10⁻³, 4×10⁻³, 4.5×10⁻³, or 5×10⁻³ Pa s.

Methods can further comprises evacuating the liquid from the cavity of the flow cell. In some instances, the liquid in the cavity of a flow cell is evacuated through the first opening. In some instances, the liquid is evacuated from the cavity by introducing air to the cavity, for example, through the second opening. In some instances, cavity is evacuated at a rate (evacuation rate) of about 0 to 50 uL/s, as described herein. In some instances during evacuation, air is introduced through a single opening (e.g., referred to as the second opening) at the top of the flow cell. As air is introduced to the cavity, liquid can be evacuated out of the cavity of the flow cell. In some instances, air is introduced to, or liquid is evacuated from the cavity through a plurality of holes. In some instances, the fluid removed the biomolecules from the substrate, and is subsequently evacuated. Thus, in some instances, the liquid comprises a plurality of biomolecules, thereby extracting a plurality of biomolecules from a substrate.

The cavity or the one or more manifolds or tubes connected to the cavity may comprise or be in contact with a heating element. Thus, in some instances, exposing the substrate placed in the cavity of a flow cell to a fluid may comprise heating the cavity. In some instances, evacuating the liquid from the cavity of the flow cell may comprise heating the cavity or heating the one or more tubes or manifolds from which the liquid is evacuated. In some instances, the heating element comprises a heating jacket, a cartridge heater, or a film heater.

Methods can further comprise collecting the liquid comprising the plurality of biomolecules. In some instances, biomolecules are collected in a sample collector. In some instances, a storage stage comprises a sample collector. In some examples, the sample collector comprises an intermediate compartment for transferring biomolecules for further processing or storage. In some examples, the sample collector comprises a storage compartment for storage of the biomolecules. The biomolecules may be stored in systems and platforms, for example, for DNA data storage, such as those provided herein (e.g., FIG. 8).

### Storage

The structure may have a variety of shapes and sizes, such as those described herein (e.g., FIGS. 9A-9I). In some instances, structures for storing polynucleotides are located or placed on plates. A structure for storing the plurality of polynucleotides may be any shape or size. In some instances, the structure is substantially spherical, tubular (FIG. 9A), egg-shaped, conical, cubic, cuboid, cylindrical, wedge, hexagonal prism, square base pyramid, triangular based pyramid, triangular prism, toroid, hemisphere, helical, heart-shaped, or other shape. In some instances, shapes are configured to allow the structure to be opened or closed to the outside environment. In some instances, such closures are facilitated by welding, seals, septums, or other mechanism for restricting the movement of gases or other matter in or out of the structure. In some instances, the structure comprises holes, slots, septum, valves, or ports for addition or removal of nucleic acids, fluids, gases, or other material into or out of the structure. In some instances a structure for storing the plurality of polynucleotides comprises a cap and a body that are flush-welded together (FIG. 9B). In some instances, a structure for storing the plurality of polynucleotides comprises a removable screw-cap (FIG. 9C). In some instances, a structure comprises a septum (FIG. 9D). In some instances a structure comprises two rounded, pill-shaped halves that form a seal when one half is inserted into the other (FIG. 9E). In some instances, a structure comprises a substantially flat, disc chamber with sealable lid (FIG. 9F). In some instances, a structure comprises a box with an optionally attached lid (FIG. 9G). In some examples, the shape is a cylinder or a disk. In some examples, a cylinder or a disk shape is preferrable for automated handling and/or filing of the structures.

In some instances the chamber comprises a plurality of vials. In some instances, vials comprise borosilicate glasses, plastics, metal, or other material. In some instances, vials are placed on a an alignment tray. After filling the vial is sealed with a metallic plate aligned to enclose the open vial once filed with polynucleotides after successfully post processing.

The structure for storing the plurality of polynucleotides may have a capacity of about 0.1 to about 1.5 mL. The capacity can generally comprise a volume of the polynucleotides, storage media, or a combination thereof. The storage media may be used to stabilize the polynucleotides, which can allow for extended storage. The storage media can comprise, for example, a salt, ionic liquid, glass, silica (e.g., silicon beads), or any other suitable storage solution. In some instances, the capacity is about 0.1 mL to 0.2 mL, 0.1 mL to 0.5 mL, 0.1 mL to 0.7 mL, 0.1 mL to 1 mL, 0.1 mL to 1.2 mL, 0.1 mL to 1.5 mL, 0.2 mL to 0.5 mL, 0.2 mL to 0.7 mL, 0.2 mL to 1 mL, 0.2 mL to 1.2 mL, 0.2 mL to 1.5 mL, 0.5 mL to 0.7 mL, 0.5 mL to 1 mL, 0.5 mL to 1.2 mL, 0.5 mL to 1.5 mL, 0.7 mL to 1 mL, 0.7 mL to 1.2 mL, 0.7 mL to 1.5 mL, 1 mL to 1.2 mL, 1 mL to 1.5 mL, or 1.2 mL to 1.5 mL. In some instances, the capacity is about 0.1 mL, 0.2 mL, 0.5 mL, 0.7 mL, 1 mL, 1.2 mL, or 1.5 mL. In some instances, the capacity is at least about 0.1 mL, 0.2 mL, 0.5 mL, 0.7 mL, 1 mL, or 1.2 mL. In some instances, the capacity is at most about 0.2 mL, 0.5 mL, 0.7 mL, 1 mL, 1.2 mL, or 1.5 mL.

The structure for storing the plurality of polynucleotides may have an internal storage volume of about 10 to about 500 microliters (μL). The internal storage volume can generally comprise a volume of the polynucleotides. In some instances, the internal storage volume is about 10 μL to 50 μL, 10 μL to 100 μL, 10 μL to 150 μL, 10 μL to 200 μL, 10 μL to 250 μL, 10 μL to 300 μL, 10 μL to 250 μL, 10 μL to 400 μL, 10 μL to 450 μL, 10 μL to 500 μL, 50 μL to 100 μL, 50 μL to 150 μL, 50 μL to 200 μL, 50 μL to 250 μL, 50 μL to 300 μL, 50 μL to 250 μL, 50 μL to 400 μL, 50 μL to 450 μL, 50 μL to 500 μL, 100 μL to 150 μL, 100 μL to 200 μL, 100 μL to 250 μL, 100 μL to 300 μL, 100 μL to 250 μL, 100 μL to 400 μL, 100 μL to 450 μL, 100 μL to 500 μL, 150 μL to 200 μL, 150 μL to 250 μL, 150 μL to 300 μL, 150 μL to 250 μL, 150 μL to 400 μL, 150 μL to 450 μL, 150 μL to 500 μL, 200 μL to 250 μL, 200 μL to 300 μL, 200 μL to 250 μL, 200 μL to 400 μL, 200 μL to 450 μL, 200 μL to 500 μL, 250 μL to 300 μL, 250 μL to 250 μL, 250 μL to 400 μL, 250 μL to 450 μL, 250 μL to 500 μL, 300 μL to 250 μL, 300 μL to 400 μL, 300 μL to 450 μL, 300 μL to 500 μL, 250 μL to 400 μL, 250 μL to 450 μL, 250 μL to 500 μL, 400 μL to 450 μL, 400 μL to 500 μL, or 450 μL to 500 μL. In some instances, the internal storage volume is about 10 μL, 50 μL, 100 μL, 150 μL, 200 μL, 250 μL, 300 μL, 250 μL, 400 μL, 450 μL, or 500 μL. In some instances, the internal storage volume is at least about 10 μL, 50 μL, 100 μL, 150 μL, 200 μL, 250 μL, 300 μL, 250 μL, 400 μL, or 450 μL. In some instances, the internal storage volume is at most about 50 μL, 100 μL, 150 μL, 200 μL, 250 μL, 300 μL, 250 μL, 400 μL, 450 μL, or 500 μL.

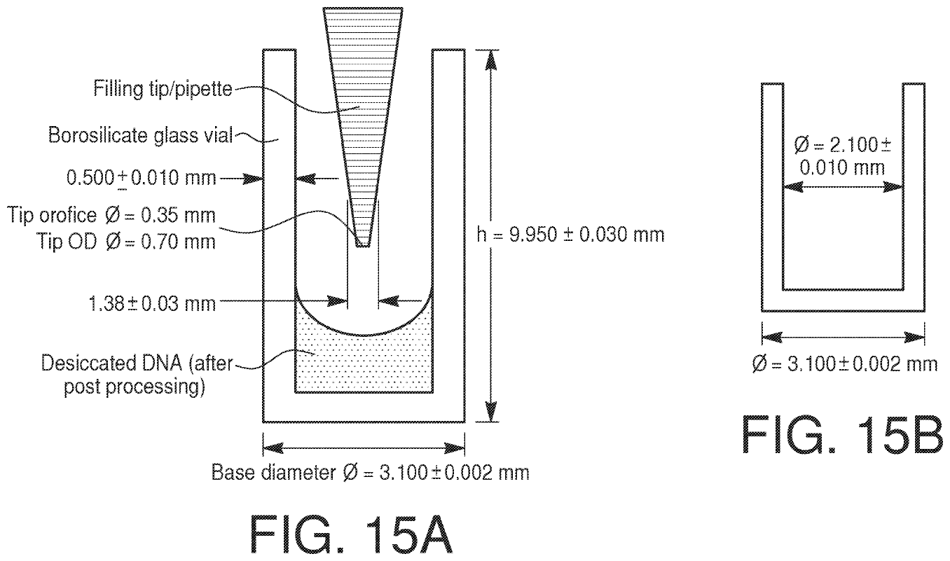

The structure for storing the plurality of polynucleotides may have a diameter of about 1 mm to about 10 mm. In some instances, the diameter is about 1 mm to 2 mm, 1 mm to 3 mm, 1 mm to 4 mm, 1 mm to 5 mm, 1 mm to 6 mm, 1 mm to 7 mm, 1 mm to 8 mm, 1 mm to 9 mm, 1 mm to 10 mm, 2 mm to 3 mm, 2 mm to 4 mm, 2 mm to 5 mm, 2 mm to 6 mm, 2 mm to 7 mm, 2 mm to 8 mm, 2 mm to 9 mm, 2 mm to 10 mm, 3 mm to 4 mm, 3 mm to 5 mm, 3 mm to 6 mm, 3 mm to 7 mm, 3 mm to 8 mm, 3 mm to 9 mm, 3 mm to 10 mm, 4 mm to 5 mm, 4 mm to 6 mm, 4 mm to 7 mm, 4 mm to 8 mm, 4 mm to 9 mm, 4 mm to 10 mm, 5 mm to 6 mm, 5 mm to 7 mm, 5 mm to 8 mm, 5 mm to 9 mm, 5 mm to 10 mm, 6 mm to 7 mm, 6 mm to 8 mm, 6 mm to 9 mm, 6 mm to 10 mm, 7 mm to 8 mm, 7 mm to 9 mm, 7 mm to 10 mm, 8 mm to 9 mm, 8 mm to 10 mm, or 9 mm to 10 mm. In some instances, the diameter is about 1 mm, 2 mm, 3 mm, 4 mm, 5 mm, 6 mm, 7 mm, 7.5 mm, 8 mm, 9 mm, or 10 mm. In some instances, the diameter is at least about 1 mm, 2 mm, 3 mm, 4 mm, 5 mm, 6 mm, 7 mm, 7.5 mm, 8 mm, or 9 mm. In some instances, the diameter is at most about 2 mm, 3 mm, 4 mm, 5 mm, 6 mm, 7 mm, 7.5 mm, 8 mm, 9 mm, or 10 mm. Referring to FIGS. 15A-15B, the diameter may be a base diameter or an internal diameter. In an exemplary embodiment, the structure or compartment for storing polynucleotides can comprise, for example, an internal diameter of about 2 mm and a base diameter of about 3 mm.

The structure for storing the plurality of polynucleotides may have a length of about 8 mm to 30 mm. In some instances, the length is about 8 mm to 10 mm, 8 mm to 12 mm, 8 mm to 15 mm, 8 mm to 18 mm, 10 mm to 20 mm, 8 mm to 22 mm, 8 mm to 25 mm, 8 mm to 28 mm, 8 mm to 30 mm, 10 mm to 12 mm, 10 mm to 15 mm, 10 mm to 18 mm, 10 mm to 20 mm, 10 mm to 22 mm, 10 mm to 25 mm, 10 mm to 28 mm, 10 mm to 30 mm, 12 mm to 15 mm, 12 mm to 18 mm, 12 mm to 20 mm, 12 mm to 22 mm, 12 mm to 25 mm, 12 mm to 28 mm, 12 mm to 30 mm, 15 mm to 18 mm, 15 mm to 20 mm, 15 mm to 22 mm, 15 mm to 25 mm, 15 mm to 28 mm, 15 mm to 30 mm, 18 mm to 20 mm, 18 mm to 22 mm, 18 mm to 25 mm, 18 mm to 28 mm, 18 mm to 30 mm, 20 mm to 22 mm, 20 mm to 25 mm, 20 mm to 28 mm, 20 mm to 30 mm, 22 mm to 25 mm, 22 mm to 28 mm, 22 mm to 30 mm, 25 mm to 28 mm, 25 mm to 30 mm, or 28 mm to 30 mm. In some instances, the length is about 10 mm, 12 mm, 15 mm, 18 mm, 20 mm, 22 mm, 25 mm, 28 mm, or 30 mm. In some instances, the length is at least about 10 mm, 12 mm, 15 mm, 18 mm, 20 mm, 22 mm, 25 mm, or 28 mm. In some instances, the length is at most about 12 mm, 15 mm, 18 mm, 20 mm, 22 mm, 25 mm, 28 mm, or 30 mm. Referring to FIGS. 15A-15B, in an exemplary embodiment, the structure or compartment for storing polynucleotides can comprise a length or height of about 10 mm.

A structure for storing a plurality of polynucleotides can comprise a variety of properties. In some instances, the structure is heat-resistant, water-resistant, corrosion-resistant, gas-tight, biologically inert, or any combination thereof. In some instances, the structure comprises a metal or organic polymer. In some instances, the structure comprises steel (e.g., stainless steel) or other metal alloy. In some instances, the structure comprises polyethylene, polypropylene, or other polymer. In some instances, the structure comprises an aramid. In some instances, the structure comprises a meta-aramid or a para-aramid. In some instances, the structure comprises a flexible material. Exemplary flexible materials include, without limitation, modified nylon, unmodified nylon, nitrocellulose, polypropylene, and carbon fiber (e.g., carbon fiber composite). In some instances, the structures comprise a rigid material. Exemplary rigid materials include, without limitation, glass, fuse silica, silicon, silicon dioxide, silicon nitride, plastics (for example, polytetrafluoroethylene, polypropylene, polystyrene, polycarbonate, and blends thereof, and metals (for example, steel, gold, platinum). In some instances, structures disclosed herein may be fabricated from a material comprising silicon, polystyrene, agarose, dextran, cellulosic polymers, polyacrylamides, polydimethylsiloxane (PDMS), glass, or any combination thereof. In some examples, structures disclosed herein are manufactured with a combination of materials listed herein or any other suitable material known in the art.

A structure described herein can comprise a material having a range of tensile strength. Exemplary materials having a range of tensile strengths include, but are not limited to, nylon (70 MPa), nitrocellulose (1.5 MPa), polypropylene (40 MPa), silicon (268 MPa), polystyrene (40 MPa), agarose (1-10 MPa), polyacrylamide (1-10 MPa), polydimethylsiloxane (PDMS) (3.9-10.8 MPa). In some instances, the material is a high tensile strength material (e.g., aramid). In some instances, the structure has a tensile strength of about 400, 600, 800, 1000, 1200, 1400, 1600, 1800, 2000, 2200, 2400, 2600, 2800, 3000, 3200, 3400, 3600, 3800, or 4000 megapascals (MPa). In some instances, the structure has a tensile strength of at least about 400, 600, 800, 1000, 1200, 1400, 1600, 1800, 2000, 2200, 2400, 2600, 2800, 3000, 3200, 3400, 3600, 3800, or 4000 megapascals (MPa). In some instances, the structure has a tensile strength of at most about 400, 600, 800, 1000, 1200, 1400, 1600, 1800, 2000, 2200, 2400, 2600, 2800, 3000, 3200, 3400, 3600, 3800, or 4000 megapascals (MPa).

Young's modulus measures the resistance of a material to elastic (recoverable) deformation under load. Exemplary materials having a range of Young's modulus stiffness include, but are not limited to, nylon (3 GPa), nitrocellulose (1.5 GPa), polypropylene (2 GPa), silicon (150 GPa), polystyrene (3 GPa), agarose (1-10 GPa), polyacrylamide (1-10 GPa), polydimethylsiloxane (PDMS) (1-10 GPa). Structures described herein can have a Young's moduli from 1 to 500, 1 to 40, 1 to 10, 1 to 5, or 3 to 11 GPa. Structures described herein can have a Young's moduli of about 1, 1.5, 2, 3, 4, 5, 6, 7, 8, 9, 10, 11, 20, 25, 40, 50, 60, 70, 80, 90, 100, 150, 200, 250, 400, 500 GPa, or more. As the relationship between flexibility and stiffness are inverse to each other, a flexible material has a low Young's modulus and changes its shape considerably under load. In some instances, a structure described herein has a surface with a flexibility of at least nylon.

Provided herein are structures for storing polynucleotides that are corrosion-resistant. Corrosion in some instances includes uniform corrosion, pitting, crevice corrosion, galvanic, stress corrosion cracking, inter-granular corrosion, corrosion caused by temperature, atmospheric corrosion, or microbial corrosion. Corrosion-resistance may be imparted by various coatings or the materials used to manufacture the structures themselves. Exemplary materials comprise metals and alloys which include iron, steel, copper, nickel, ferrous alloys (e.g., stainless steel, alloys with chromium, such as at least 10.5% chromium, or other amount), copper-based alloys, and nickel-based alloys. In some instances, corrosion resistance is imparted by a sacrificial anode. In some instances, a structure described herein comprises a rare earth metal. In some instances, a structure described herein comprises platinum, palladium, gold, silver, rhodium, titanium, or other corrosion-resistant material. In some instances, a coating of the structure comprises a glass. The glass coating may be used in addition to, or as an alternative to a metallic coating, such as those described herein.

The corrosion resistance of a structure described herein may be measured through various means known in the art. For example, corrosion resistance is measured using tests provided by the American Society for Testing and Materials (ASTM). Exemplary protocol categories for measuring corrosion resistance include corrosion of metals in natural atmospheric, aqueous environments and electrochemical measurements in corrosion testing, laboratory corrosion tests, or other standard test. Exemplary corrosion tests include but are not limited to salt spray, modified salt spray (acetic acid salt spray, acidified salt fog, seawater acidified, SO₂ salt spray, or dilute electrolyte salt fog/dry Test). Test samples are then examined for evidence of corrosion through various means such as weight loss, loss of thickness, examination of the surface (pitting/crevice), electrochemical measurements, functionality, or other method. In an exemplary measurement, the loss in thickness due to corrosion of a structure described herein is expressed as MPY (mils per year, wherein 1 mil=0.0254 mm). In some instances, MPY=87.6×(metal density in g/cm³×area of sample in cm²×time of exposure in hours). In some instances, a corrosion-resistant structure described herein has an MPY of less than 1 when exposed to sea water at 25 degrees C., or an MPY of less than 0.001, 0.01, 0.05, 0.1, 0.2, 0.5, 0.7, 1, 1.2, 1.5, 2, 5, or less than 10. In some instances, a corrosion-resistant structure described herein has an MPY of less than 1 when exposed to a 1 percent (w/w) acetic acid solution at 100 degrees C., or an MPY of less than 0.1, 0.5, 1, 5, 10, 20, or less than 50.

Provided herein are structures for storing polynucleotides that are gas-tight. Such structures may be used to reduce or eliminate exposure of polynucleotides to harmful/degrading elements of the outside environment. Gas-tight in some instances refers to the resistance or impermeability to gas transfer from the environment inside the structure to the environment outside of the structure, which reduces or eliminates contact of gases with stored polynucleotides, or maintains a pressure or vacuum inside the structure. Gas-tight structures in some instances maintain a constant pressure due to welds, seals, septum, or other sealing mechanism. In some instances, a gas-tight structure is capable of holding an internal pressure of at least 1 atm, 1.5, 2, 3.5, 10, 20, or at least 50 atm. In some instances, a gas-tight structure is capable of holding a vacuum. In some instances, a gas-tight structure is capable of holding an internal pressure (vacuum) of at least 100 torr, at least 10 torr, 1 torr, 0.1 torr, 0.01 torr, 0.001 torr, 0.0001 torr, 0.00001 torr, or at least 0.000001 torr. In some instances, the gas inside the gas-tight structure is an inert gas. In some instances, the inert gas is nitrogen, argon, helium, or a mixture thereof.

A structure for storing a plurality of polynucleotides encoding for digital information may comprise components such as sensors. Sensors often provide information regarding the status of the structure, including environmental information. Therefore, one or more sensors can be used to monitor the data storage system In some instances, sensors provide data regarding open/closed state or position, locked/unlocked state, atmospheric contents (water content, oxygen content), temperature, history (last time opened or closed) or other information. This information is optionally used to monitor the proper storage or health of polynucleotides contained inside a structure described herein. In some instances, a sensor comprises one or more control polynucleotides that are sequenced to verify fidelity of the bulk of the digital information encoded in the structure. In some instances, these control polynucleotides do not comprise digital information. In some instances, a sensor indicating damage or a lack of fidelity is indicative of errors in the digital information encoded therein. In some examples, the plurality of polynucleotides are reviewed by wholly or partially sequencing them to determine errors. In some instances, a structure comprising a sensor that indicates damage or a lack of fidelity to one or more polynucleotides is flagged for review or discarded. In some instances, a structure comprising a sensor that indicates damage or a lack of fidelity to one or more polynucleotides is erased and re-written with polynucleotides encoding for the original or error-free digital information. In some examples, the one or more sensors comprises a temperature sensor, a humidity sensor, a pressure sensor, a salinity sensor, a light sensor, a UV sensor, an O₂ sensor, or any combination thereof. The one or more sensors can be used to monitor the environment inside, outside, or both of the structure. The sensor information can be transmitted back to a controller in a data storage system to maintain integrity of the plurality of polynucleotides. In some instances, the one or more sensors is integrated with a tag for indexing as described herein. In some instances, the one or more sensors is integrated with an RFID tag in a structure. In some examples, an RFID reader provides an alert if the one or more sensors is outside of a threshold.

A structure for storing a plurality of polynucleotides encoding for digital information may comprise coatings on one or more surfaces. In some instances, coatings are present on the inside surface of a structure (not in contact with the outside environment when the structure is closed/sealed). In some instances, coatings are present on the outside surface of a structure (exposed to environment). A structure in some instances comprises one or more coatings, such as 1, 2, 3, 4, 5, or more than 5 coatings. Coatings in some instances comprise similar materials, or alternatively at least some of the coatings comprise different materials. Various coatings in some instances provide one or more properties to the surface of the structure such as increased resistance to corrosion, desiccation, hydrophobicity, oxygen absorption, or other property conducive to polynucleotide storage. Exemplary coatings include but are not limited to coatings that comprise plastics, synthetic polymers, glass, silica, metals, biological polymers, proteins, or other material.

A structure for storing polynucleotides encoding for digital information may comprise a means for indexing the content therein. The means for indexing may be used to identifying the structure, or contents therein. The means for indexing can comprise a tag, as previously described herein. The structure may comprise a tag as provided, for example, in FIGS. 9H-9I. The tag can comprise a barcode or an RFID tag. In some instances, the tag is superficial to the structure (FIG. 9H). In some instances, the tag is embedded in the structure (FIG. 9I). In some instances, the tag cannot be removed. In some instances, the tag is impossible to remove. In some instances, the tag cannot be altered. In some instances, the tag is impossible to alter. In some instances, the tag comprises a barcode, an RFID tag, a nucleic acid barcode, a protein, a small molecule tag, or other means for uniquely identifying the structure from other structures. In some instances, the tag is an RFID tag. In some examples, the RFID tag is an active RFID tag or a passive RFID tag as described herein. In some examples, the RFID tag is a UHF RFID tag, a HF RFID tag, or a LF RFID tag, as described herein. In some instances, a database is used to store information regarding a structure's contents by associating such a structure to its means for indexing (e.g., tag). In some instances, a database is not used to store information regarding a structure's contents. In such instances, the tag is used to store information regarding a structure's contents. In some examples, an RFID tag is used to store metadata relating to the structure's content (e.g., plurality of polynucleotides, digital information, etc.).

The tag on the structure can be about 0.1 mm² to about 5 mm². In some instances, the size of the tag is 0.1 mm² to 0.5 mm², 0.1 mm² to 1 mm², 0.1 mm² to 2 mm², 0.1 mm² to 3 mm², 0.1 mm² to 4 mm², 0.1 mm² to 5 mm², 0.5 mm² to 1 mm², 0.5 mm² to 2 mm², 0.5 mm² to 3 mm², 0.5 mm² to 4 mm², 0.5 mm² to 5 mm², 1 mm² to 2 mm², 1 mm² to 3 mm², 1 mm² to 4 mm², 1 mm² to 5 mm², 2 mm² to 3 mm², 2 mm² to 4 mm², 2 mm² to 5 mm², 3 mm² to 4 mm², 3 mm² to 5 mm², or 4 mm² to 5 mm². In some instances, the size of the tag is 0.1 mm², 0.5 mm², 1 mm², 2 mm², 3 mm², 4 mm², or 5 mm². In some instances, the size of the tag is at least 0.1 mm², 0.5 mm², 1 mm², 2 mm², 3 mm², or 4 mm². In some instances, the size of the tag is at most 0.5 mm², 1 mm², 2 mm², 3 mm², 4 mm², or 5 mm².

The tag on the structure can have a thickness of about 0.1 mm to about 3 mm. In some instances, the thickness is 0.1 mm to 0.5 mm, 0.1 mm to 1 mm, 0.1 mm to 1.5 mm, 0.1 mm to 2 mm, 0.1 mm to 3 mm, 0.5 mm to 1 mm, 0.5 mm to 1.5 mm, 0.5 mm to 2 mm, 0.5 mm to 3 mm, 1 mm to 1.5 mm, 1 mm to 2 mm, 1 mm to 3 mm, 1.5 mm to 2 mm, 1.5 mm to 3 mm, or 2 mm to 3 mm. In some instances, the thickness is 0.1 mm, 0.5 mm, 1 mm, 1.5 mm, 2 mm, or 3 mm. In some instances, the thickness is at least 0.1 mm, 0.5 mm, 1 mm, 1.5 mm, or 2 mm. In some instances, the thickness is at most 0.5 mm, 1 mm, 1.5 mm, 2 mm, or 3 mm.

The plurality of polynucleotides in a structures may be stored according to a variety of storage mechanisms. A storage mechanism can comprise, without limitation, a dehydration mechanism, ionic solvent mechanism, salt-based containment mechanism, glass-based containment mechanism, or any combination thereof. In some instances, the plurality of polynucleotides are stored in solution or as a solid in the structure. In some instances, the structure comprises a medium for storing the plurality of polynucleotides. In some examples, the medium comprises a solid, a liquid, a gas, or any combination thereof. In some examples, the medium comprises an organic solvent. In some examples, the medium comprises water. In some examples, the medium does not comprise water. In some example, the medium comprises ethanol, isopropanol, or glycerol. In some examples, the medium comprises a salt solution. In some examples, the molar ratio of salt to DNA may range from about 20:1 to about 2:1. In some examples, the molar ratio depends on the molecular weight of the salt used and on the relative amounts of salt and DNA combined. In some examples, the molar ratio is calculated between the cation of the salt and the negatively charged phosphate groups of the DNA. In some examples, the salt solution comprises a molar ratio of less than 20:1 salt cation to phosphate groups in the DNA. In some examples, the salt solution is dried to create a dried product. In some cases, the salt solution comprises, by way of non-limiting examples, calcium chloride, calcium nitrate, calcium carbonate, calcium phosphate, magnesium chloride, magnesium sulfate, magnesium nitrate, magnesium carbonate, lanthanum chloride, lanthanum nitrate, lanthanum carbonate, lanthanum bromide, or a mixture thereof. In some instances, the salt solution comprises barium (II) chloride dihydrate, calcium chloride dihydrate, copper (II) chloride anhydrous, lanthanum trichloride, magnesium dichloride hexahydrate, sodium chloride, or strontium chloride hexahydrate. In some instances, the concentration of the salt solution is about 0.01 nM to about 0.1 nM.

In some instances, a medium for storing the plurality of polynucleotides comprises nanoparticles. In some instances, the nanoparticles comprise silica nanoparticles. In some instances, a subset of the plurality of polynucleotides are encapsulated in the nanoparticles. In some instances, the nanoparticles encapsulating polynucleotides are stored in a water-free or near-to water-free environment. In some instances, nanoparticles comprise a protective layer of silica (e.g., tetraethoxysilane). In some instances, the nanoparticles comprise a co-interacting compound with the polynucleotides (e.g., N-[3-(Trimethoxysilyl)propyl]-N,N,N-trimethylammonium chloride). In some instances, the nanoparticles encapsulating polynucleotides are stored in a structure described herein. In some instances, the nanoparticles encapsulating polynucleotides are stored on a structure, such as a digital microfluidic chip. The structure can comprise a tag, such as an RFID tag described herein. In some instances, the digital microfluidic chip allows for programmability of fluid. In some instances, the programmability allows for automated storage and/or retrieval of polynucleotides. In some instances, each location on a digital microfluidic chip comprises about 100 GB, 500 GB, 1 TB, 2 TB, 10 TB, 20 TB, 30 TB, or 50 TB. In some instances, each location comprises about 50 μg, 100 μg, 150 μg, 200 μg, 250 μg, 300 μg, 350 μg, 400 μg, 450 μg, 500 μg, 600 μg, 700 μg, 800 μg, 900 μg, or 1000 μg of nanoparticles.

In some instances, the structure for storing a plurality of polynucleotides is implemented as an article of manufacture. In some instances, an article of manufacture comprises a structure for storing a plurality of polynucleotides encoding digital information. In some instances, the structure is a high durability structure. In some instances, the structure comprises a radio frequency identification (RFID) tag, such as those described herein. In some instances, the RFID tag comprises metadata relating to the plurality of polynucleotides.

In some instances, a plurality of polynucleotides are transferred to a structure in the synthesizer unit 1810. In some instances, the plurality of polynucleotides are transferred to a structure from a flow cell, which in some instances part of a biomolecule extraction system. In some instances, a plurality of polynucleotides are transferred to a structure in the storage unit 1815. The fluidic and/or electronic control of polynucleotide synthesis in the storage unit 1815 may be performed by a controller 1835. In some instances, the electronics in the storage unit 1815 are in communication with the controller 1835. In some instances, the polynucleotides are stored at room temperature in the storage unit 1815. In some instances, the system comprises a database or a file system for keeping track of the storage capacity in the storage unit 1815. In some examples, the database comprises a control application database. In some instances, the database or the file system is part of the controller 1835.

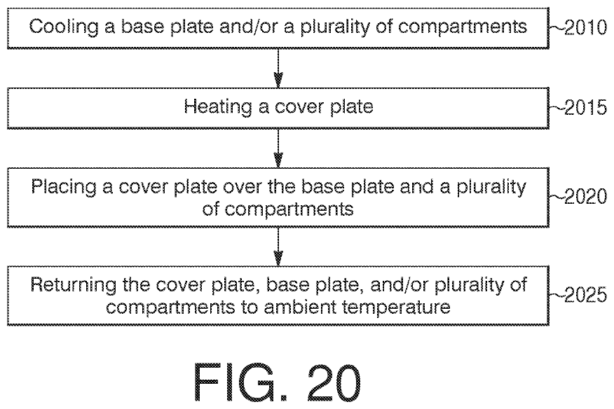

In some instances, the plurality of polynucleotides are stored in a device comprising a plurality of compartments, a base plate, and at least one cover plate as described herein. The polynucleotides may be transferred to a compartment using, for example, a material deposition system comprising a nozzle (e.g., dispenser). Sealing methods employing thermal expansion and/or contraction of one or more components of the system, or for example, thermal glue may be used to seal the compartments. The temperature gradients between or across one or more components of the device may be generated by one or more temperature control systems, such as a cooling system, a heating system, or both. The temperature control system may be used to seal the device, for example, using the workflow generally illustrated in FIG. 20.

A structure comprising a plurality of polynucleotides can be stored in an identifiable layout in storage unit 1815. The identifiable layout may comprise a rack or a plurality of racks, or a variation thereof. The rack may be used to hold one or more structures comprising the plurality of polynucleotides. In some instances, each structure is stored at a fixed location in the identifiable layout. In some instances, the tag comprises information about a location of the structure in the identifiable layout. As an example, a tag (e.g., RFID tag) can encode metadata comprising a location of the structure in the identifiable layout. In some instances, the rack may be located in a data center. In some instances, the rack uses mechanical structures commonly used for mounting conventional computing and data storage resources in rack units. For example, a rack may comprise openings adapted to support disk drives, processing blades, and/or other computer equipment. In some instances, a rack comprises a tag. In some examples, the tag comprises information of the structures stored in/on the rack. In some examples, the tag comprises a list of the structures stored in/on the rack.

In some instances, the storage unit 1815 may be accessed using a robotic system 1830. In some instances, the identifiable layout in the storage unit 1815 comprises robotically addressable slots. Each slot may hold a structure comprising a plurality of polynucleotides. In some instances, each slot comprises a width, depth, length, or any combination thereof for accommodating a structure comprising the plurality of polynucleotides. In some instances, a rack comprises a plurality of slots, where each slot holds a structure comprising the plurality of polynucleotides.

Provided herein are devices or assemblies comprising structures (e.g., compartments or containers) for storing polynucleotides described herein. Also provided herein are systems and methods using the device or assembly for storing polynucleotides. In some instances, the systems, methods, devices, or assemblies relate to long-term storage of about 20, 30, 50, or 100 years or more. The device or assembly can generally comprise one of more of: a base plate, a plurality of compartments, and a cover plate. In some instances, provided herein are methods for filling, sealing, or assembling the components into devices or assemblies for use in long term polynucleotide storage.

Devices or assemblies for storing polynucleotides can comprise a plurality of compartments. The compartments may comprise a structure for polynucleotide storage described elsewhere herein, for example, the structures illustrated in FIGS. 5-11. The compartments, in some instances, have one or more tags described herein. In some examples, the compartments comprise vials. In some examples, the compartments comprise lids. In some examples, the compartments do not comprise lids. An exemplary diagram illustrating an embodiment of a structure or compartment for storing polynucleotides is provided in FIG. 15A-15B. While the compartment in FIGS. 15A-15B illustrate a circular compartment with a diameter, in some instances, the compartments may be square or rectangular, or any other shape.

The compartment for storing polynucleotides can comprise a diameter. The diameter may be a base diameter or an internal diameter. In some instances, the internal diameter is less than the base diameter. The base diameter may be about 1 mm to about 10 mm as described elsewhere herein. In some instances, the base diameter is about 1 mm to about 5 mm. In some instances, the internal diameter may be about 1 mm to about 10 mm as described elsewhere herein. In some instances, internal diameter is about 1 mm to about 5 mm. Referring to FIGS. 15A-15B, the base diameter may be about 3.2 mm and the internal diameter may be about 2.2 mm.

The compartment for storing polynucleotides can comprise a height. The height (or length) may be about 8 mm to 15 mm as described elsewhere herein. In some instances, the height is about 8 mm to about 12 mm. In some instances, the height is less than about 10 mm. Referring to FIGS. 15A-15B, the height may be about 9.95 mm.

In some instances, the compartments or structures have a thickness. In some instances, the thickness is about 0.1 mm to about 2 mm. In some instances, the thickness is about 0.5 mm. In some instances, the thickness is 0.1 to 0.2 mm, 0.1 to 0.5 mm, 0.1 to 0.8 mm, 0.1 to 1 mm, 0.1 to 1.2 mm, 0.1 to 1.5 mm, 0.1 to 1.8 mm, 0.1 to 2 mm, 0.2 to 0.5 mm, 0.2 to 0.8 mm, 0.2 to 1 mm, 0.2 to 1.2, 0.2 to 1.5 mm, 0.2 to 1.8 mm, 0.2 to 2 mm, 0.5 to 0.8 mm, 0.5 to 1 mm, 0.5 to 1.2 mm, 0.5 to 1.5 mm, 0.5 to 1.8 mm, 0.5 to 2 mm, 0.8 to 1 mm, 0.8 to 1. mm 2, 0.8 to 1.5 mm, 0.8 to 1.8 mm, 0.8 to 2, 1 to 1.2 mm, 1 to 1.5 mm, 1 to 1.8 mm, 1 to 2 mm, 1.2 to 1.5 mm, 1.2 to 1.8 mm, 1.2 to 2 mm, 1.5 to 1.8 mm, 1.5 to 2 mm, or 1.8 to 2 mm. In some instances, the thickness is 0.1, 0.2, 0.5, 0.8, 1, 1.2, 1.5, 1.8, or 2 mm. In some instances, the thickness is at least 0.1, 0.2, 0.5, 0.8, 1, 1.2, 1.5, or 1.8 mm. In some instances, the thickness is at most 0.2, 0.5, 0.8, 1, 1.2, 1.5, 1.8, or 2 mm.

The plurality of polynucleotides may be stored in the compartment. The plurality of polynucleotide may be in a liquid or a gas. In some instances, the plurality of polynucleotides are in a liquid when they are transferred to or from the compartment(s). In some instances, the plurality of polynucleotides are in a liquid when they are stored in the compartment(s). In some instances, the plurality of polynucleotides are in a solid when they are transferred to or from the compartment(s). In some instances, the plurality of polynucleotides are in a solid when they are stored in the compartment(s). In an exemplary method, the plurality of polynucleotides are in solution when they are transferred to the compartment(s).

In some instances, each compartment of the plurality of compartments comprises a volume of a solution comprising the plurality of polynucleotides. In some examples, the volume, also referred to as internal storage volume, is about 10 μL to about 500 μL as described elsewhere herein. In some instances, the volume is about 10 μL to about 200 μL. In some instances, the volume is less than about 100 μL. In some instances, the volume is about 10 μL to about 50 μL. The plurality of polynucleotides may be dried down when they are stored (e.g., for long term storage). In some instances, the solution is dried under vacuum. In some instances, the plurality of polynucleotides are in solution when they are retrieved or accessed. In some instances, the plurality of polynucleotides are dissolved in a solution when they are retrieved.

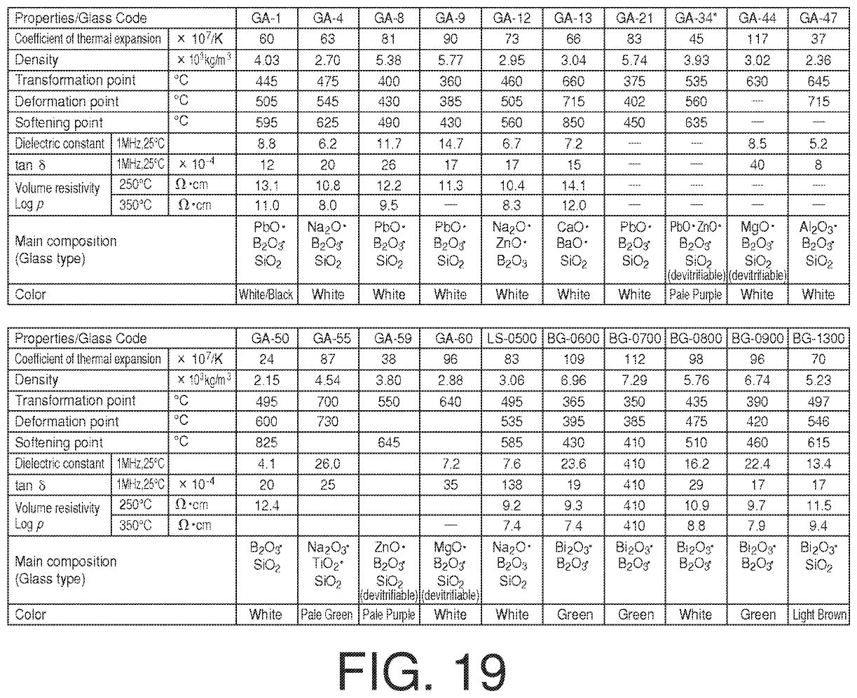

The compartment for storing polynucleotides can comprise a first material. The first material may be inert. More specifically, the first material may be biologically inert such that it does not interact with material within the compartment (e.g., polynucleotides). The compartment may comprise a variety of properties described herein, such as, but not limited to being heat-resistant, water-resistant, corrosion-resistant, or gas-tight. The material for the compartment can comprise, in some instances, a metal or an organic polymer, such as, but not limited to those described herein. In some instances, the material has a tensile strength or a Young's modulus in a range described herein. In some examples, the material comprises a glass. In some examples, the material comprises silica (or silicon dioxide). In some instances, the material comprises at least about 10%, 20%, 30%, 40%, 50%, 60%, 70%, 80%, or 90% silica. In some instances, the material further comprise a metal oxide, such as, but not limited to, a boron oxide, sodium oxide, potassium oxide, aluminum oxide, or any combination thereof. Various types of glass that may be selected for the first material is provided, for example, in FIG. 19. Referring to FIG. 15A, the compartment can comprise, in an exemplary embodiment, borosilicate. In some examples, the compartments comprise glass vials.

In some instances, the plurality of compartments are positioned on the base plate. In some instances, the plurality of compartments are arranged on the base plate to facilitate storing or retrieving the polynucleotides from the plurality compartments. For example, the plurality of compartments may be arranged to facilitate filling the compartments with DNA suspended in a solution using dispenser, such as a nozzle or pipette, or drying down the DNA. FIG. 15A provides an example illustrating a nozzle that can be used to deliver or remove material from the compartment.

In some instances, the dispenser (e.g., nozzle, pipette, etc.) is part of a material deposition system. The material deposition system can have a plurality of dispenser (e.g., plurality of nozzles). In some instances, a dispenser for filling the compartments is different than a dispenser for removing the content of the compartments. In some instances, one or more dispensers for filling the compartments, one or more nozzles for removing the content of the compartments, or both, are part of the material deposition system.

The dispenser (e.g., nozzle, pipette, etc.) may have various dimensions suitable for use with the compartments described herein. In some instances, a dispenser width is about 0.5 to about 5 mm. In some instances, a dispenser width is 0.5 to 1, 0.5 to 2, 0.5 to 3, 0.5 to 4, 0.5 to 5, 1 to 2, 1 to 3, 1 to 4, 1 to 5, 2 to 3, 2 to 4, 2 to 5, 3 to 4, 3 to 5, or 4 to 5 mm. In some instances, a dispenser width is 0.5, 1, 2, 3, 4, or 5 mm. In some instances, a dispenser width is at least 0.5, 1, 2, 3, or 4 mm. In some instances, a dispenser width is at most 1, 2, 3, 4, or 5 mm. Referring to FIG. 15A, the nozzle width is, in an exemplary embodiment, about 1.38 mm. However, this width may be varied based at least in part on the internal diameter of the compartment. In some instances, the tip of the dispenser comprises a diameter. The diameter may be an internal diameter (referred to as tip orifice in FIG. 15A) or an outer diameter (OD). In some instances, the diameter is about 0.1 mm to about 1 mm. In some instances, the diameter is 0.1 to 0.2 mm, 0.1 to 0.3 mm, 0.1 to 0.4 mm, 0.1 to 0.5 mm, 0.1 to 0.6 mm, 0.1 to 0.7 mm, 0.1 to 0.8 mm, 0.1 to 0.9 mm, 0.1 to 1 mm, 0.2 to 0.3 mm, 0.2 to 0.4 mm, 0.2 to 0.5 mm, 0.2 to 0.6 mm, 0.2 to 0.7 mm, 0.2 to 0.8 mm, 0.2 to 0.9 mm, 0.2 to 1 mm, 0.3 to 0.4 mm, 0.3 to 0.5 mm, 0.3 to 0.6 mm, 0.3 to 0.7 mm, 0.3 to 0.8 mm, 0.3 to 0.9 mm, 0.3 to 1 mm, 0.4 to 0.5 mm, 0.4 to 0.6 mm, 0.4 to 0.7 mm, 0.4 to 0.8 mm, 0.4 to 0.9 mm, 0.4 to 1 mm, 0.5 to 0.6 mm, 0.5 to 0.7 mm, 0.5 to 0.8 mm, 0.5 to 0.9 mm, 0.5 to 1 mm, 0.6 to 0.7 mm, 0.6 to 0.8 mm, 0.6 to 0.9 mm, 0.6 to 1 mm, 0.7 to 0.8 mm, 0.7 to 0.9 mm, 0.7 to 1 mm, 0.8 to 0.9 mm, 0.8 to 1 mm, or 0.9 to 1 mm. In some instances, the diameter is 0.1, 0.2, 0.3, 0.4, 0.5, 0.6, 0.7, 0.8, 0.9, or 1 mm. In some instances, the diameter is at least 0.1, 0.2, 0.3, 0.4, 0.5, 0.6, 0.7, 0.8, or 0.9 mm. In some instances, the diameter is at most 0.2, 0.3, 0.4, 0.5, 0.6, 0.7, 0.8, 0.9, or 1 mm. Referring to FIG. 15A, in an exemplary embodiment, the internal diameter is about 0.35 mm or the outer diameter is about 0.7 mm. However, the internal diameter, the outer diameter, or both may be varied based at least in part on the dimensions of the compartment or material being filled or removed from the compartment (e.g., viscosity of the solution).

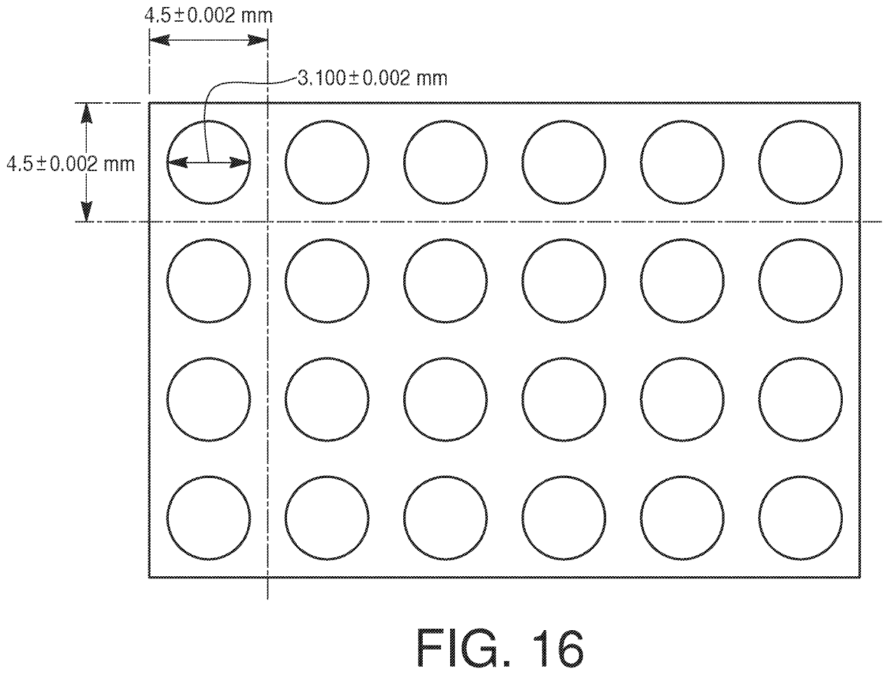

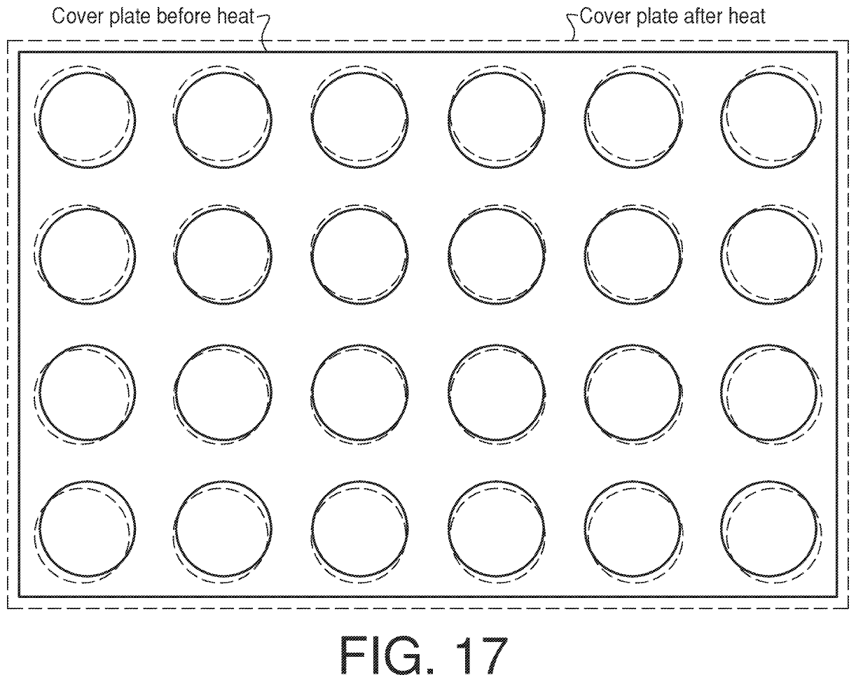

In some instances, the plurality of compartments are positioned in an array on the base plate. An exemplary schematic of a base plate is shown in FIG. 16. The base plate can comprise a plurality of recessed features. In some instances, the recessed features may also be referred to as tapped holes. The plurality of recessed features of the base plate may have a diameter is about 1 mm to about 10 mm as described elsewhere herein. In some instances, the plurality of recessed features of the base plate have a diameter of about 1 mm to about 5 mm. In some instances, each of the plurality of recessed features of the base plate has the same diameter as the base diameter of a compartment of the plurality of compartments.

The plurality of compartments may be positioned in the recessed features of the base plate. The plurality of recessed features of the base plate may form a well comprising a wall. In some instances, each compartment is located wholly or partially within a recessed feature of the base plate. In some instances, the plurality of compartments are integrated into the base plate. In some instances, the plurality of compartments and the base plate are in communication, interact, or interoperate by way of mechanical or electronic means. In some instances, the plurality of compartments are separable from the base plate.

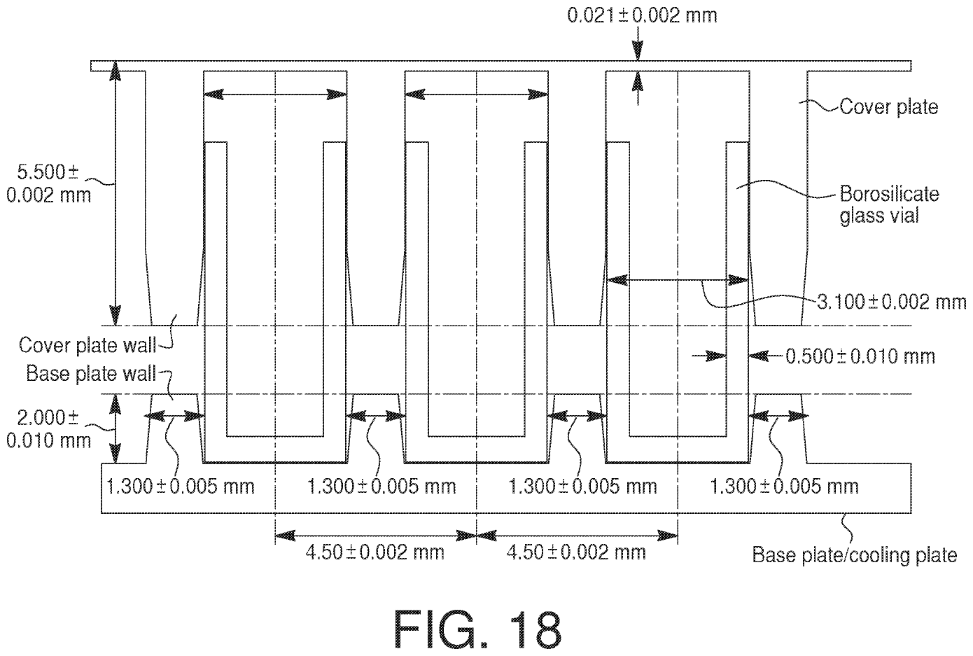

In some instances, the wall formed by the recessed feature is about 1 mm to about 10 mm in length. In some instances, the wall is 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10 mm in length. In some instances, the wall is 1, 2, 3, 4, 5, 6, 7, 8, 9, or 10 mm in length. In some instances, the wall is at least 1, 2, 3, 4, 5, 6, 7, 8, or 9 mm in length. In some instances, the wall is at most 2, 3, 4, 5, 6, 7, 8, 9, or 10 mm in length. As shown for example in FIG. 18, the wall may be about 2 mm in length.

In some instances, each wall of the recessed features is at least partially angled, as shown for example in FIG. 18. In some instances, each wall of the recessed feature of the base plate is at least partially angled at about 0.1° to 1°. In some instances, each wall of the recessed feature of the base plate is at least partially angled at no more than 1°. In some instances, each wall of the recessed feature of the base plate is partially angled at about 0.1°, 0.2°, 0.3°, 0.4°, 0.5°, 0.6°, 0.7°, 0.8°. 0.9°, or 1°. In some instances, each wall of the recessed feature of the base plate is partially angled at least about 0.1°, 0.2°. 0.3°, 0.4°, 0.5°, 0.6°. 0.7°, 0.8°, 0.9°, or 1°. In some instances, each wall of the recessed feature of the base plate is partially angled at most about 0.1°, 0.2°, 0.3°, 0.4°, 0.5°, 0.6°, 0.7°, 0.8°, 0.9°, or 1°. In some instances, each wall of the recessed feature of the base plate is partially angled at 0.1° to 0.2°, 0.1° to 0.3°, 0.1° to 0.4°, 0.1° to 0.5°, 0.1° to 0.6°, 0.1° to 0.7°, 0.1° to 0.8°, 0.1° to 0.9°, 0.1° to 1°, 0.2° to 0.3°, 0.2° to 0.4°, 0.2° to 0.5°, 0.2° to 0.6°, 0.2° to 0.7°, 0.2° to 0.8°, 0.2° to 0.9°, 0.2° to 1°, 0.3° to 0.4°, 0.3° to 0.5°. 0.3° to 0.6°, 0.3° to 0.7°, 0.3° to 0.8°, 0.3° to 0.9°, 0.3° to 1°, 0.4° to 0.5°, 0.4° to 0.6°, 0.40 to 0.7°. 0.40 to 0.8°, 0.40 to 0.9°, 0.40 to 1°, 0.50 to 0.6°, 0.50 to 0.7°, 0.50 to 0.8°, 0.50 to 0.9°, 0.5° to 1°, 0.6° to 0.7°, 0.6° to 0.8°, 0.6° to 0.9°, 0.6° to 1°, 0.7° to 0.8°, 0.7° to 0.9°, 0.7° to 1°, 0.8° to 0.9°, 0.8° to 1°, or 0.9° to 1°.

In some instances, each wall between the recessed features has a thickness of about 0.5 mm to about 3 mm. In some instances, the thickness is 0.5 to 1, 0.5 to 1.5, 0.5 to 2, 0.5 to 2.5, 0.5 to 3, 1 to 1.5, 1 to 2, 1 to 2.5, 1 to 3, 1.5 to 2, 1.5 to 2.5, 1.5 to 3, 2 to 2.5, 2 to 3, or 2.5 to 3 mm. In some instances, the thickness is about 0.5, 1, 1.5, 2, 2.5, or 3 mm. In some instances, the thickness is at least about 0.5, 1, 1.5, 2, or 2.5 mm. In some instances, the thickness is at most about 1, 1.5, 2, 2.5, or 3 mm. Referring to FIG. 18, in an exemplary embodiment, the thickness is about 1.3 mm.

The recessed features can have a pitch distance. In some instances, the recessed features of the base plate have a uniform pitch. In some instances, the pitch distance is about 1 to about 15 mm. In some instances, the pitch distance is 1 to 2 mm, 1 to 3 mm, 1 to 4 mm, 1 to 5 mm, 1 to 6 mm, 1 to 7 mm, 1 to 8 mm, 1 to 9 mm, 1 to 10 mm, 1 to 12 mm, 1 to 15 mm, 2 to 3 mm, 2 to 4 mm, 2 to 5 mm, 2 to 6 mm, 2 to 7 mm, 2 to 8 mm, 2 to 9 mm, 2 to 10 mm, 2 to 12 mm, 2 to 15 mm, 3 to 4 mm, 3 to 5 mm, 3 to 6 mm, 3 to 7 mm, 3 to 8 mm, 3 to 9. mm 3 to 10 mm, 3 to 12 mm, 3 to 15, 4 to 5, 4 to 6 mm, 4 to 7 mm, 4 to 8 mm, 4 to 9 mm, 4 to 10 mm, 4 to 12, 4 to 15 mm, 5 to 6 mm, 5 to 7 mm, 5 to 8 mm, 5 to 9 mm, 5 to 10 mm, 5 to 12 mm, 5 to 15 mm, 6 to 7 mm, 6 to 8 mm, 6 to 9 mm, 6 to 10 mm, 6 to 12 mm, 6 to 15 mm, 7 to 8 mm, 7 to 9 mm, 7 to 10 mm, 7 to 12 mm, 7 to 15 mm, 8 to 9 mm, 8 to 10 mm, 8 to 12 mm, 8 to 15 mm, 9 to 10 mm, 9 to 12 mm, 9 to 15 mm, 10 to 12 mm, 10 to 15 mm, or 12 to 15 mm. In some instances, the pitch distance is 1, 2, 3, 4, 5, 6, 7, 8, 9, 10, 12, or 15 mm. In some instances, the pitch distance is at least 1, 2, 3, 4, 5, 6, 7, 8, 9, 10, or 12 mm. In some instances, the pitch distance is at most 2, 3, 4, 5, 6, 7, 8, 9, 10, 12, or 15 mm. Referring to FIG. 16 or FIG. 18, in an exemplary embodiment, the pitch distance is about 4.5 mm.

The base plate can comprise a second material. In some instances, the second material is biologically inert. In some instances, the second material comprises a metal. In some instances, the second material comprises a high specific heat capacity material. The high specific heat capacity material may comprise a specific heat capacity generally about 0.5 J/k-° C. to 2.5 J/k-° C. In some instances, the specific heat capacity is about 0.5, 1, 1.5 or 2 J/k-° C. In some instances, the specific heat capacity is at least about 0.5, 1, 1.5 or 2 J/k-° C. In some instances, the specific heat capacity is at most about 0.5, 1, 1.5 or 2 J/k-° C. In some instances, the specific heat capacity is about 0.5 to 1, 0.5 to 1.5, or 0.5 to 2, 1 to 1.5, 1 to 2, or 1.5 to 2 J/k-° C. A high specific heat capacity material may comprise, by way of non-limiting example, aluminum, beryllium, or magnesium. In some instances, the base plate comprises a metallic mounting plate, a cooling plate, or both.

The device can further comprise a cover plate. In some instances, the cover plate comprises a plurality of recessed features. The plurality of recessed features (also may be referred to as tapped holes) may correspond to the same positions as the plurality of recessed features of the base plate. In some instances, the plurality of recessed features on the base plate are arranged in an array, and the plurality of recessed features on the cover plate are arranged in an array. Each of the plurality of recessed features of the cover plate may fit the plurality of compartments, for example, as provided in FIG. 18. In some instances, a diameter of each of the recessed features of the cover plate is the same or similar to the base diameter of each of the compartments. In some instances, a diameter of each of the recessed features of the cover plate is the same or similar to the base diameter of each of the compartments at about 15° C. to 30° C. or at about room temperature.

An exemplary schematic of FIG. 16 can also correspond to the cover plate. The plurality of recessed features in the cover plate may have a diameter is about 1 mm to about 10 mm as described elsewhere herein. In some instances, the plurality of recessed factures of the cover plate have a diameter of about 1 mm to about 5 mm. In some instances, each of the plurality of recessed features of the cover plate has the same or similar diameter as the base diameter of a compartment of the plurality of compartments. The recessed features of the cover plate can have a pitch distance, as similarly described for the base plate. In some instances, the recessed features of the cover plate have a uniform pitch. In some instances, the pitch distance is about 1 to about 15 mm as described. Referring to FIG. 16 or FIG. 18, in an exemplary embodiment, the diameter of each of the recessed features of the cover plate is about 3.195 mm, or the pitch distance is about 4.5 mm, or both.

In some instances, the plurality of the recessed features of the cover plate form a well comprising a wall. In some instances, each wall of the recessed features is at least partially angled, as shown for example in FIG. 18. In some instances, each wall of the recessed feature of the base plate is at least partially angled at about 0.10 to 1° as described herein. In some instances, each wall of the recessed feature of the base plate is at least partially angled at no more than 1° as described herein. In some examples, the walls of the recessed features of the cover plate are partially angled at a same or similar angle to the walls of the recessed features of the base plate. In some instances, each wall between the recessed features of the cover plate has a thickness of about 0.5 mm to about 3 mm as described herein. In some examples, the thickness of the walls between the recessed features of the cover plate are the same or similar to the thickness of the walls between the recessed features of the base plate. Referring to FIG. 18, in an exemplary embodiment, the walls may be partially angled at less than 0.5°, the thickness of the walls between the recessed features of the cover plate is about 1.3 mm, or both. Further, the wall formed by the recessed features of the cover plate may be about 1 mm to about 10 mm in length as described herein. In some instances, the wall formed by the recessed features of the cover plate may be taller than the wall formed by the recessed features of the base plate. The thickness of the top surface of the recessed features of the cover plate may be about 0.005 to about 0.1 mm. In some instances, the thickness of the top surface is about 0.005 to 0.01, 0.005 to 0.02, 0.005 to 0.05, 0.005 to 0.08, 0.005 to 0.1, 0.01 to 0.02, 0.01 to 0.05, 0.01 to 0.08, 0.01 to 0.1, 0.02 to 0.05, 0.02 to 0.08, 0.02 to 0.1, 0.05 to 0.08, 0.05 to 0.1, or 0.08 to 0.1 mm. In some instances, the thickness of the top surface is 0.005, 0.01, 0.02, 0.05, 0.08, or 0.1 mm. In some instances, the thickness of the top surface is at least 0.005, 0.01, 0.02, 0.05, or 0.08 mm. In some instances, the thickness of the top surface is at most 0.01, 0.02, 0.05, 0.08, or 0.1 mm. Referring to FIG. 18, in an exemplary embodiment, the wall formed by the recessed features of the cover plate is about 5.5 mm, the thickness of the cover plate at the top surface of the recessed features is about 0.021 mm, or both.

The cover plate may be placed on the plurality of compartments such that the plurality of compartments may be fully or partially housed by the walls of the cover plate, the walls of the base plate, or both. In some instances, walls of each of the recessed features of the cover plate and an outer wall of the compartment are separated by about 1 μm to about 10 μm. In some instances, walls of each of the recessed features of the cover plate and an outer wall of the compartment are separated by about 1 μm, 2 μm, 3 μm, 4 μm, 5 μm, 6 μm, 7 μm, 8 μm, 9 μm, or 10 μm. In some instances, walls of each of the recessed features of the cover plate and an outer wall of the compartment are separated by at least about 1 μm, 2 μm, 3 μm, 4 μm, 5 μm, 6 μm, 7 μm, 8 μm, 9 μm, or 10 μm. In some instances, walls of each of the recessed features of the cover plate and an outer wall of the compartment are separated by at most about 1 μm, 2 μm, 3 μm, 4 μm, 5 μm, 6 μm, 7 μm, 8 μm, 9 μm, or 10 μm. In some instances, walls of each of the recessed features of the cover plate and an outer wall of the compartment are separated by about 1-2, 1-3, 1-4, 1-5, 1-6, 1-8, 1-10, 2-3, 2-4, 2-5, 2-6, 2-8, 2-10, 3-4, 3-5, 3-6, 3-8, 3-10, 4-5, 4-6, 4-8, 4-10, 5- 8, 5-10, 6-8, 6-10, 7-9, 7-10, 8-10, or 9-10 μm. In some instances, walls of each of the recessed features of the cover plate and the outer wall of each of the compartments are separated by no more than 5 μm at about 15° C. to 30° C. or at about room temperature.

The cover plate can comprise a third material. In some instances, the third material is biologically inert. In some instances, the third material comprises a metal. In some instances, the third material comprises low emissivity. In some instances, the third material comprises stainless steel. In some instances, the third material comprises a coefficient of thermal expansion (CTE) of about 10 μm/m-° C. to 25 μm/m-° C. In some instances, the third material comprises a CTE of about 15 μm/m-° C. to 20 μm/m-° C. In some instances, the third material comprises a CTE of 17 μm/m-° C. In some instances, the third material comprises a CTE of about 10, 11, 12, 13, 14, 15, 16, 17, 18, 19, 20, 21, 22, 23, 24, or 25 μm/m-° C. In some instances, the third material comprises a CTE of about 10-12, 10-15, 10-18, 10-20, 10-25, 12-15, 12-18, 12-20, 12-25, 15-18, 15-20, 15-22, 15-25, 18-20, 18-22, 18-25, 20-22, or 20-25 μm/m-° C. In some instances, the first material of the compartments, the second material of the base plate, or both, have a CTE of less than the CTE of the third material of the cover plate. In some instances, the second material of the base plate and the third material of the cover plate have the same or a similar CTE. In some instances, the first material of the plurality of compartments have a CTE less than the third material of the cover plate.

In some instances, the third material comprises a thermal conductivity of about 10 W/m-K to about 25 W/m-K. In some instances, the third material comprises a thermal conductivity of about 15 W/m-K to about 20 W/m-K. In some instances, the third material comprises a thermal conductivity of 16.2 W/m-K. In some instances, the third material comprises a thermal conductivity of about 10, 11, 12, 13, 14, 15, 16, 17, 18, 19, 20, 21, 22, 23, 24, or 25 W/m-K. In some instances, the third material comprises a thermal conductivity of about 10-12, 10-15, 10-18, 10-20, 10-25, 12-15, 12-18, 12-20, 12-25, 15-18, 15-20, 15-22, 15-25, 18-20, 18-22, 18-25, 20-22, or 20-25 W/m-K.

In some instances, the third material comprises a specific heat capacity of about 0.1 to 1 J/g-° C. In some instances, the third material comprises a specific heat capacity of about 0.1, 0.2, 0.3, 0.4, 0.5, 0.6, 0.7, 0.8, 0.9, or about 1 J/g-° C. In some instances, the third material comprises a specific heat capacity of about 0.1-0.2, 0.1-0.5, 0.1-0.8, 0.1-1, 0.2-0.5, 0.2-0.8, 0.2-1, 0.3-0.5, 0.3-0.8, 0.3-1, 0.4-0.6, 0.4-0.8, 0.4-1, 0.5-0.8, 0.5-1, 0.6-0.8, 0.6-1, 0.7-0.9, 0.7-1, 0.8-1, or 0.9-1 J/g-° C. In some instances, the third material comprises a specific heat capacity of 0.5 J/g-° C.

The device or assembly can comprise at least one cover plate. In some instances, the device comprises more than one cover plate. In some instances, the device comprises a plurality of cover plates. In some instances, the device comprise about one, two, three, four, five, six, seven, eight, nine, tcn, 15, 20, 30, 40, 50, 60, 70, 80, 90, 100, 120, 150, or 200 cover plates. In some instances, the device comprise at least about one, two, three, four, five, six, seven, eight, nine, ten, 15, 20, 30, 40, 50, 60, 70, 80, 90, 100, 120, 150, or 200 cover plates. In some instances, the device comprise at most about one, two, three, four, five, six, seven, eight, nine, ten, 15, 20, 30, 40, 50, 60, 70, 80, 90, 100, 120, 150, or 200 cover plates. In some instances, the device comprises about 1-10, 1-50, 1-100, 10-50, 10-80, 10-100, 40-80, 40-100, 40-120, 50-80, 50-100, 50-120, 50-200, 60-100, 60-150, 80-100, 80-120, 80-150, 80-200, 100-120, 100-150, 100-200, or 150-200 cover plates. In some instances, the device comprises about 64 cover plates. In some instances, the device comprises about 96 cover plates. In some instances, each compartment of the plurality of compartments has its own cover plate. In some instances, each cover plate covers about one, two, three, four, five, six, ten, 12, 16, 24, 30, 36, 40, 48, or 96 compartments of the plurality of compartments. In some instances, each cover plate covers at least about one, two, three, four, five, six, ten, 12, 16, 24, 30, 36, 40, 48, or 96 compartments of the plurality of compartments. In some instances, each cover plate covers at most about one, two, three, four, five, six, ten, 12, 16, 24, 30, 36, 40, 48, or 96 compartments of the plurality of compartments. In some instances, each cover plate covers about 1-5, 1-10, 1-12, 1-20, 1-50, 1-100, 2-5, 2-10, 2-15, 2-20, 2-50, 4-10, 4-20, 4-50, 5-10, 5-20, 5-30, 5-50, 10-15, 10-20, 10-50, 10-100, 20-40, 20-50.20-100, 40-50, 40-100, 50-80, or 50-100 compartments of the plurality of compartments. In some instances, the cover plate covers all compartments of the device. In some instances, the cover plate covers all compartments arranged on a base plate. In some instances, the device comprises at least two cover plates. In some examples, each of the at least two cover plates seal about six compartments of the plurality of compartments. In some instances, the cover plate and the base plate are the same size. In some instances, the cover plate is about 1%, 2%, 3%, 4%, 5/a, 8%, 10%, 20%, 30%, 40%, 50%, 60%, 70%, 80%, 90%, or 100% the size of the base plate. In some instances, the cover plate is about 1-5%, 2-8%, 5-10%, 8-20%, 15-30%, 20-40%, 30-50%, 40-60%, 50-70%, 60-80%, 70-90%, or 80-100% the size of the base plate.

Any dimension of a component in the device for storing polynucleotides (e.g., a compartment or structure, base plate, or cover plate) can comprise a tolerance. The dimension may be any dimension described or illustrated herein, such as, by way of non-limiting example, a diameter (e.g., base diameter, internal diameter), height, length, thickness, pitch distance, or any combination thereof. In some instances, any one of the dimensions of a compartment can comprise a dimensional tolerance of from about one sigma to six sigma. In some instances, any one of the dimensions of a compartment can comprise a dimensional tolerance of up to about three sigma. In some instances, the dimensional tolerance of a dimension of the compartment is about one to two, one to three, one to four, one to five, one to six, two to three, two to four, two to five, two to six, three to four, three to five, three to six, four to five, four to six, or about five to six sigma. In some instances, the dimensional tolerance of a dimension of the compartment is about one, two, three, four, five, or six sigma. In some instances, the dimensional tolerance of a dimension of the compartment is at most about one, two, three, four, five, or six sigma. In some instances, the dimension comprises, for example, a diameter, pitch distance, length, or thickness described herein. In some examples, the dimension comprises a tolerance of about 10 μm or less. In some instances, the dimension comprises a tolerance of about 10, 8, 5, 4, 3, 2, or 1 μm or less. In some instances, the dimension comprises a tolerance of about 1-2, 1-3, 1-4, 1-5, 1-6, 1-8, 1-10, 2-3, 2-4, 2-5, 2-8, 2-10, 3-5, 3-8, 3-10, 4-8, 4-10, 5-8, 5-10, 6-10, or 8-10 μm.

The device or assembly as described herein comprising a base plate, a plurality of compartments, and a cover plate may be used to store information. The information may be encoded in DNA. A method for storing information may comprise providing the device comprising one or more of: a plurality of compartments, a base plate; and a cover plate. In some instances, the plurality of compartments comprise a plurality of polynucleotides. In some examples, the plurality of compartments may be a plurality of glass vials. In some instances, the base plate, the plurality of compartments, or both are not in contact with the cover plate. The plurality of compartments may be arranged on the base plate (or mounting plate) in an array to facilitate filling or drying down the material in the compartments. In some instances, the method comprises transferring the plurality of polynucleotides to the compartment. In some instances, transferring comprises transferring the plurality of polynucleotides in a solution. In some examples, synthetic DNA suspended in a liquid solution is transferred to a compartment using a dispenser, such as a nozzle or a pipette. The dispense may be part of a material deposition system as described further herein. Thus, in some instances, transferring comprises depositing one or more droplets comprising the plurality of polynucleotides using a nozzle of a deposition system.

Each compartment of the plurality of compartments may be filled with a volume of a solution comprising the plurality of polynucleotides. The volume may be the same, similar to, or less than the internal volume of the compartments of the plurality of compartments. Thus, in some instances, a compartment is filled with a volume of about 10 μL to about 500 μL. In some instances, a compartment is filled with a volume of about 10 μL to about 200 μL. In some instances, the volume is less than about 100 μL. In some instances, the volume is about 10 μL to about 50 μL.

The contents of the compartments (e.g., DNA) may be stored in solution or as a solid. In some examples, the contents of the compartments are dried. e.g., by vacuum. In some instances, the drying the contents leaves the desiccated DNA in the bottom of compartment.

The method can comprise positioning the cover plate above the base plate and/or the plurality of compartments. The cover plate may be positioned above the base plate and/or the plurality of compartments using a piezo stage, a vision system, linear motor, rotary motor, any combination thereof, or any other technique known in the art.

Methods for sealing the compartments in the device or assembly provided herein may generally comprise using temperature. A temperature seal may utilize, for example, thermal contraction and/or thermal expansion of a material of a component of the device (e.g., base plate, cover plate, or compartment) or a thermal glue for sealing one or more components of the device.

The method generally comprises generating a temperature gradient across components of the device in order to seal the contents of the compartments. In some instances, a temperature gradient is generated between the base plate and the cover plate. In some instances, a temperature gradient causes the base plate or the cover plate to expand or contract. In some instances, the expansion or contraction of one or more components of the device can seal the compartments and the contents therein. In some examples, generating the temperature gradient comprises cooling or heating the base plate and/or the compartments therein, the cover plate, or both. In some examples, generating the temperature gradient comprises sequentially changing a temperature of the base plate and/or the compartments therein, and the cover plate. In some examples, generating the temperature gradient comprises simultaneously changing a temperature of the base plate and/or the compartments therein, and the cover plate.

In some instances, generating a temperature gradient comprises one or more operations. An exemplary workflow of the one or more operations is generally illustrated in FIG. 20. The one or more operations may be performed simultaneously or sequentially. In some instances, the one or more operation comprises cooling the base plate 2010. The base plate, which may comprise a mounting plate and/or a cooling plate may be cooled in an inert atmosphere. In some instances, the plurality of compartments, which may be arranged on the base plate, are also cooled. The base plate may be cooled to a predetermined temperature. In some instances, the base plate is cooled to about −100 degrees Celsius to about 100 degrees Celsius. In some examples, the base plate is cooled to about −50 degrees Celsius to about 50 degrees Celsius. In some instances, the base plate is cooled to about −100° C., −90° C., −80° C., −70° C., −60° C., −50° C., −40° C., −30° C., −20° C., −10° C., −5° C., 0° C., 5° C., 10° C., 15° C., 20° C., 25° C., 30° C., 40° C., 50° C., 60° C., 70° C., 80° C., 90° C., or about 100° C. In some instances, the base plate is cooled to at least about −100° C., −90° C., −80° C., −70° C., −60° C., −50° C., −40° C., −30° C., −20° C., −10° C., −5° C., 0° C., 5° C., 10° C., 15° C. 20° C., 25° C., 30° C. 40° C., 50° C., 60° C. 70° C., 80° C., 90° C. or about 100° C. In some instances, the base plate is cooled to at most about −100° C., −90° C., −80° C., −70° C., −60° C., −50° C., −40° C., −30° C., −20° C., −10° C., −5° C., 0° C., 5° C., 10° C., 15° C. 20° C., 25° C., 30° C., 40° C., 50° C., 60° C., 70° C., 80° C., 90° C., or about 100° C. In some instances, the base plate is cooled to at most about −100° C. to −80° C., −100° C. to −50° C., −100° C. to −20° C., −100° C. to 0° C., −80° C. to −50° C., −80° C. to −20° C., −80° C. to 0° C., −80° C. to 20° C., −50° C. to −20° C., −50° C. to −10° C., −50° C. to 0° C., −50° C. to 10° C., −50° C. to 20° C., −30° C. to −20° C., −30° C. to −10° C., −30° C. to 0° C., −30° C. to 10° C., −30° C. to 20° C., −20° C. to −10° C., −20° C. to −5° C., −20° C. to 0° C., −20° C. to 5° C., −20° C. to 10° C., −20° C. to 20° C., −10° C. to −5° C., −10° C. to 0° C., −10° C. to 5° C., −10° C. to 10° C., −10° C. to 15° C., −10° C. to 20° C., −10° C. to 25° C., −5° C. to 0° C., −5° C. to 5° C., −5° C. to 10° C., −5° C. to 15° C., −5° C. to 20° C., −5° C. to 25° C., 0° C. to 5° C. 0° C. to 10° C., 0° C. to 15° C., 0° C. to 20° C., 0° C. to 25° C., 0° C. to 30° C., 5° C. to 10° C., 5° C. to 15° C., 5° C. to 20° C., 5° C. to 25° C., 5° C. to 30° C., 5° C. to 40° C., 10° C. to 15° C., 10° C. to 20° C. 10° C. to 25° C., 10° C. to 30° C., 10° C. to 40° C., 15° C. to 20° C., 15° C. to 25° C. 15° C. to 30° C., 15° C. to 40° C., 15° C. to 50° C. 20° C. to 25° C., 20° C. to 30° C., 20° C. to 40° C., 20° C. to 50° C., 25° C. to 30° C. 25° C. to 40° C., 25° C. to 60° C., 25° C. to 80° C., 30° C. to 40° C. 30° C. to 50° C., 30° C. to 80° C., 50° C. to 80° C., 50° C. to 100° C., 70° C. to 80° C., 70° C. to 100° C., or 80° C. to 100° C. In some instances, the base plate or the plurality of components contracts by about 1% to 50%, In some examples, the base plate or the plurality of components contracts by about 1% to 25%, In some instances, the base plate or the plurality of components contracts by 0.01%, 0.05%, 0.1%, 0.2%, 0.5%, 0.8%, 1%, 1.2%, 1.5%, 1.8%, 2%, 2.5%, 3%, 3.5%, 4%, 4.5%, 5%, 6%, 7%, 8%, 9%, 10%, 15%, 20%, 25%, 30%, 40%, or 50%. In some instances, the base plate or the plurality of components contracts by at least about 0.01%, 0.05%, 0.1%, 0.2%, 0.5%, 0.8%, 1%, 1.2%, 1.5%, 1.8%, 2%, 2.5%, 3%, 3.5%, 4% 4%, 5%, 6%, 7%, 8%, 9%, 10%, 15%, 20%, 25%, 30%, 40%, or 50%. In some instances, the base plate or the plurality of components contracts by at most about 0.01%, 0.05%, 0.1%, 0.2%, 0.5%, 0.8%, 1%, 1.2%, 1.5%, 1.8%, 2%, 2.5%, 3%, 3.5%, 4a %, 4.5%, 5%, 6%, 7%, 8%, 9%, 10%, 15%, 20%, 25%, 30%, 40%, or 50%, In some instances, the base plate or the plurality of components contracts by 0.01% to 0.05%, 0.01% to 0.1%, 0.01% to 0.2%, 0.01% to 0.5%, 0.05% to 0.1%, 0.05% to 0.2%, 0.05% to 0.5%, 0.05% to 0.8°%, 0.1% to 0.2%, 0.1% to 0.5%, 0.1% to 0.8°%, 0.1% to 1%, 0.1% to 1.2%, 0.1% to 1.5%, 0.2% to 0.5%, 0.2% to 0.8%, 0.2% to 1%, 0.2% to 1.2%, 0.2% to 1.5%, 0.5% to 0.8°%, 0.5% to 1%, 0.5% to 1.2%, 0.5% to 1.5%, 0.5% to 1.8%, 0.5% to 2%, 0.5% to 2.5%, 0.8% to 1%, 0.8% to 1.2%, 0.8% to 1.5%, 0.8% to 1.8%, 0.8% to 2%, 0.8% to 2.5%, 1% to 1.2%, 1% to 1.5%, 1% to 1.8°%, 1% to 2.1% to 2.5%, 1.2% to 1.5%, 1.2% to 1.8%, 1.2% to 2%, 1.2% to 2.5%, 1.5% to 1.8%, 1.5% to 2%, 1.5% to 2.5%, 1.8% to 2%, 1.8% to 2.5%, 2% to 2.5%, 2% to 3%, 2% to 4%, 2% to 5%, 2% to 8%, 2% to 10%, 3% to 4%, 3% to 5%, 3% to 8%, 3% to 10%, 3% to 15%, 3% to 20%, 3% to 25%, 4% to 5%, 4% to 8%, 4% to 10%, 4% to 15%, 4% to 20%, 5% to 8%, 5% to 10%, 5% to 15%, 5% to 20/%, 5% to 25%, 5% to 30%, 5% to 50%, 8% to 10%, 8% to 15%, 8% to 20%, 8% to 25%, 8% to 50%, 10% to 15%, 10% to 20%, 10% to 25%, 10% to 30%, 10% to 40%, 10% to 50%, 15% to 20%, 15% to 25%, 15% to 30%, 15% to 40%, 15% to 50%, 20% to 25%, 20% to 30%, 20% to 40%, 20% to 50%, 25% to 30%, 25% to 40%, 25% to 50%, 30% to 40%, 30% to 50%, or 40% to 50%.

In some instances, the one or more operation comprises heating the cover plate 2015. The cover plate may be a metallic cover plate, e.g., stainless steel, as described herein. In some instances, heating the cover plate causes the cover plate to expand, for example, as illustrated in FIG. 17. The cover plate may be heated to a predetermined amount, for example, enough to cover the plurality of compartments arranged on the base plate. In some instances, the cover plate is heated to about 0 degrees Celsius to about 250 degrees Celsius. In some examples, the cover plate is heated to about 20 degrees Celsius to about 100 degrees Celsius. In some instances, the cover plate is heated to about 0° C., 10° C., 20° C., 30° C., 40° C., 50° C., 60° C., 70° C., 80° C., 90° C., 100° C., 120° C., 150° C., 180° C., 200° C., 220° C. or 250° C. In some instances, the cover plate is heated to at least about 0° C., 10° C., 20° C., 30° C., 40° C., 50° C., 60° C., 70° C., 80° C., 90° C., 100° C., 120° C., 150° C., 180° C., 200° C., 220° C., or 250° C. In some instances, the cover plate is heated to at most about 0° C., 10° C., 20° C., 30° C., 40° C., 50° C., 60° C., 70° C., 80° C., 90° C., 100° C., 120° C., 150° C., 180° C., 200° C., 220° C., or 250° C. In some instances, the cover plate is heated to about 0° C., to 10° C., 0° C., to 20° C., 0° C., to 30° C., 0° C., to 40° C., 0° C., to 50° C., 0° C., to 70° C., 0° C., to 90° C., 10° C., to 20° C., 10° C., to 30° C., 10° C., to 40° C., 10° C., to 50° C., 10° C., to 80° C., 10° C., to 100° C., 20° C., to 30° C., 20° C., to 40° C., 20° C., to 50° C., 20° C., to 80° C., 20° C., to 100° C., 30° C., to 40° C., 30° C., to 50° C., 30° C., to 70° C., 30° C., to 90° C., 40° C., to 50° C., 40° C., to 60° C., 40° C., to 80° C., 40° C., to 100° C., 50° C., to 60° C., 50° C., to 80° C., 50° C., to 100° C., 60° C., to 70° C., 60° C., to 90° C., 60° C., to 120° C., 70° C., to 80° C., 70° C., to 90° C., 70° C., to 120° C., 80° C., to 100° C., 80° C., to 120° C., 80° C. to 150° C., 90° C., to 100° C., 90° C., to 120° C., 90° C., to 150° C., 100° C., to 120° C., 100° C. to 150° C., 100° C., to 180° C., 100° C., to 200° C., 100° C., to 220° C., 100° C., to 250° C., 120° C., to 150° C., 120° C., to 180° C., 120° C., to 200° C., 120° C., to 220° C., 120° C., to 250° C., 150° C., to 180° C., 150° C., to 200° C., 150° C. to 220° C., 150° C., to 250° C., 180° C., to 200° C., 180° C., to 220° C., 180° C., to 250° C., 200° C., to 220° C., 200° C., to 250° C., or 220° C., to 250° C. In some instances, the cover plate expands by about 1% to 50%, In some examples, the cover plate expands by about 1% to 25%, In some instances, the cover plate expands by 0.01%, 0.05%, 0.1%, 0.2%, 0.5%, 0.8%, 1%, 1.2%, 1.5%, 1.8%, 20%, 2.5%, 3%, 3.5%, 4%, 4.5%, 5%, 6%, 7%, 8%, 9%, 100%, 15%, 20%, 25%, 30%, 40%, or 50%. In some instances, the cover plate expands by at least 0.01%, 0.05%, 0.1% a, 0.2%, 0.5%, 0.8%, 1%, 1.2%, 1.5%, 1.8%, 2%, 2.5%, 3%, 3.5%, 4%, 4.5% 5%, 6%, 7%, 8%, 9%, 10%, 15%, 20%, 25%, 30%, 40%, or 50%, In some instances, the cover plate expands by at most 0.01%, 0.05%, 0.1%, 0.2%, 0.5%, 0.80%, 1%, 1.2%, 1.5%, 1.8%, 20%, 2.5%, 3%, 3.5%, 4%, 4.5%, 5%, 6%, 7%, 8%, 9%, 10%, 15%, 20%, 25%, 30%, 40%, or 50%, In some instances, the cover plate expands by 0.01% to 0.05%, 0.01% to 0.1%, 0.01% to 0.2%, 0.01% to 0.5%, 0.05% to 0.1%, 0.05% to 0.2%, 0.05% to 0.5%, 0.05% to 0.8%, 0.1% to 0.2%, 0.1% to 0.5%, 0.1% to 0.8%, 0.1% to 1%, 0.1% to 1.2%, 0.1% to 1.5%, 0.2% to 0.5%, 0.2% to 0.8%, 0.2% to 1%, 0.2% to 1.2%, 0.2% to 1.5%, 0.5% to 0.8%, 0.5% to 1%, 0.5% to 1.2%, 0.5% to 1.5%, 0.5% to 1.8%, 0.5% to 2%, 0.5% to 2.5%, 0.8% to 1%, 0.8% to 1.2%, 0.8% to 1.5%, 0.8% to 1.8%, 0.8% to 2%, 0.8% to 2.5%, 1% to 1.2%, 1% to 1.5%, 1% to 1.8%, 1% to 2%, 1% to 2.5%, 1.2% to 1.5%, 1.2% to 1.8%, 1.2% to 2%, 1.2% to 2.5%, 1.5% to 1.8%, 1.5% to 2%, 1.5% to 2.5%, 1.8% to 2%, 1.8% to 2.5%, 2% to 2.5%, 2% to 3%, 2% to 4%, 2% to 5%, 2% to 8%, 2% to 10%, 3% to 4%, 3% to 5%, 3% to 8%, 3% to 10%, 3% to 15%, 3% to 20%, 3% to 25%, 4% to 5%, 4% to 8%, 4% to 104, 4% to 15%, 4% to 20%, 5% to 8%, 5% to 10%, 5% to 15%, 5% to 20%, 5% to 25%, 5% to 30%, 5% to 50%, 8% to 10%, 8% to 15%, 8% to 20%, 8% to 25%, 8% to 50%, 10% to 15%, 10% to 20%, 10% to 25%, 10% to 30%, 10% to 40%, 10% to 50%, 15% to 20%, 15% to 25%, 15% to 30%, 15% to 40%, 15% to 50%, 20% to 25%, 20% to 30%, 20% to 40%, 20% to 50%, 25% to 30%, 25% to 40%, 25% to 50%, 30% to 40%, 30% to 50%, or 40% to 50%.

Once the temperature of the cover plate, the base plate (and/or the compartments), or both have been equilibrated, the heating of the cover plate and/or the cooling of the base plate (and/or the compartments) may be stopped. The cover plate may then be placed on or pushed down onto the plurality of compartments 2020. In some instances, depending on the expansion of the cover plate, it may not be necessary to cool the base plate or the plurality of compartments. The cover plate, the base plate, and/or the plurality of compartments may be returned to a predetermined temperature 2025. The predetermined temperature may be the storage temperature, such as ambient temperature or less. The cover plate may contract or shrink around the plurality of compartments (e.g., thermal contraction), thereby sealing the compartments and the content (e.g., polynucleotides or DNA) therein.

The temperature gradient generated across one or more components of the device or assembly, for example in the operations of FIG. 20, may be monitored. The temperature may be monitored radiantly, inductively, or resistively. In some instances, the temperature gradient is monitored by a sensor for measuring one or more of temperature, emission, or resistance, such as those described herein. In some examples, a sensor is part of a system comprising the device, or is part of the one or more compartments of the device, such as for example, the base plate, the cover plate, or a compartment or structure described herein.

The polynucleotides or a portion thereof may be retrieved from the device described herein. Thus, in some instances, the method further comprises retrieving the polynucleotides or a portion thereof. In some instances, retrieving comprises removing the cover plate in contact with the plurality of containers, the base plate, or both. In some examples, retrieving comprises heating the cover plate or a portion thereof, cooling the base plate or a portion thereof, or both. In some examples, heating the cover plate or a portion thereof causes the cover plate to expand and/or cooling the base plate or portion thereof causes the base plate to contract, such that the cover plate can be removed from the plurality of compartments.

In some instances, the plurality of polynucleotides or a portion thereof may be retrieved without removing the cover plate in contact with the plurality of compartment and/or the base plate. In some instances, retrieving comprises piercing a portion of the cover plate. The cover plate may be pierced, for example, using a needle or a syringe, or any other suitable instrument known in the art. In some examples, if the polynucleotides are stored as a solid, the instrument, such as a needle or syringe may be used to dissolve the plurality of polynucleotides in a solution before retrieving it or a portion thereof from the compartment.

Selection of dimensions or materials of the device for encapsulation described herein may comprise one or more considerations. In some instances, selection of the dimensions of the cover plate, base plate (e.g., cooling plate), or compartments (e.g., glass vials) creates a strong compression seal between the cover plate and the outer edges of the compartments (e.g., sides of the glass vials). In some examples, this can hermetically seal the contents of the compartments. In some instances, as the heating or cooling processes, or the sealing process, are conducted in an inert atmosphere, the polynucleotides or DNA in the compartments is protected from degradation, for example, due to impurities sealed into the final structure or ingress of external impurities over time. In some instances, by selection of the cover plate material and compartment material (e.g., glass vials) such that they have similar temperature coefficients of expansions (TCEs), the integrity of the compression seal can be maintained over a relatively wide range of temperatures. In some instances, by selecting the cover plate materials, and/or specifically the thickness of the cover plate over the top of the compartments, the top of the cover plate may be pierced to access the DNA. In some examples, piercing a portion of the top of the cover plate may enable rehydration and/or aliquoting for subsequent processing (e.g. amplification, sequencing, etc.).

In some instances, the accessed DNA can be decoded and information encoded in the DNA can be recovered.

The dimensions and materials for encapsulation described here may illustrate an exemplary embodiment. In some instances, the dimensions and materials for encapsulation described here facilitate incorporation with standard biotechnology components and systems known in the art. In some instances, the dimensions and materials for encapsulation described here illustrate the method and/or demonstrate the functionality. However, the materials selected and/or dimensions illustrated in exemplary embodiments may not preclude the utilization of other materials and/or dimensions.

Operation of a successful embodiment of the encapsulation system, methods, devices, or assemblies described herein may be dependent on a number of factors. In some instances, a factor comprises the dimensional tolerances of one or more components (e.g., base plate, cover plate, or compartments), the materials selected for these components, the assembly of the system (e.g., used for positioning, heating, cooling, moving and locating the individual components), or any combination thereof. In some examples, the 3 sigma tolerances of the compartment (e.g., vial) wall thickness and the outer dimension(s) of the compartments are within the combination of the tolerances of the recessed features (e.g., tapped holes) in the cover plate and the control of the upper temperature that the plate is heated to. In some examples, while some movement of the vials when positioning the heated cover plate is feasible (e.g., given the elasticity and soft locations of the components), the cover plate may be registered accurately and hence positioning feedback systems may be used when pushing the cover plate onto the vials. In some examples, materials, tolerances, or dimensions may be selected such that a small separation (e.g., micron to few microns) is achieved between the recessed features (e.g., tapped holes) in the cover plate and outer edge of the compartment(s) (e.g., vial(s)). In such examples, premature cooling and compression of the cover plate may be avoided. In some examples, deleterious heating of the desiccated DNA may be avoided by limiting the maximum temperature the cover plate is heated to. In some further examples, deleterious heating of the desiccated DNA may be avoided by reducing its emissivity to the maximum extent possible. In some examples, the compartments comprise a borosilicate glass with a high thermal conductivity. In some examples, the base plate and/or compartments may be cooled to a temperature consistent with eliminating cracking (e.g., through differential contraction) of the glass vial as the cover plate is positioned.

In some instances, one or more considerations discussed herein are addressed by selection of the materials used to manufacture the one or more components and/or the dimensions and number of components used. In some examples, the number of components comprises a number of components per unit cell. For example, due to the expansion on heating of the cover plate, the larger the number of compartments (e.g., vials) per cover plate, the greater the offset may be between compartment position and recessed feature (e.g., tapped hole) on the cover plate during heating and expansion. While in some examples, for periodicities of 6 vials, this angle may be small (e.g., about 0.45°) larger cover plates may result in greater offsets in alignment between the base plate/cooling plate and the cover plate. In some examples, the exemplary embodiments illustrated herein may adjusted, for example, for higher cover plate heating (e.g., by shielding and modification of the compartment geometry), adjustment of the compartment sizes and shapes, modifications to the cooling plate geometry, or orientation or even different numbers and pitches of recessed features (e.g., wells) and compartments (e.g., vials) encapsulated.

### Content Indexing

Provided herein are systems and methods for content indexing for DNA data storage. A system for storing digital information may comprise a plurality of polynucleotides. The plurality of polynucleotides can be collectively encoding digital information. A system of storing digital information can further comprise a structure for storing the plurality of polynucleotides. The structure can comprise a means for indexing based on content stored in the structure. In some examples, the means for indexing comprises a tag, such as a radio frequency identification (RFID) tag. The tag can comprise, for example, metadata relating to the content, such as the plurality of polynucleotides or the digital information stored in the structure. In some instances, the tag can further comprise an association between the digital information encoded in the plurality of polynucleotides and an external file system or database. In some instances, a tag may be a label, a marker, an identifier, or any variation thereof. A tag may be part of a structure for polynucleotide storage, such as those provided herein.

A tag can comprise one or more features. The one or more features can allow the tag to serve as a label, a file system, a database, or a combination thereof. In some instances, the file system is a dynamic file system. In some cases, the one or more features comprises remote accessibility. In some instances, the entire content or part of the content of one or more structure for DNA data storage is remotely catalogued within and/or after removal of the structure from a storage system. In some cases, the one or more features comprises identification and/or selection of individual structures. In some instances, an individual structure is identified and/or selected from a plurality of structures based on the tag for sequencing. In some cases, the one or more features comprises knowledge of the disposition of all or part of the structures in the storage system. In some instances, the disposition of the structures in the storage system is knowable in real-time by a system control software. In some instances, the disposition of the structures is presented to a system host application. In some cases, the one or more features comprises the ability to update the tag. In some instances, after the contents of a structure has been copied (e.g., PCR) and sequenced to access the digital information, the tag can be updated to reflect access to the contents in the structure. In some cases, the one or more features comprises security. In some instances, a tag is incorporated in the structure such that the provenance of the plurality of polynucleotides in the capsule is indisputable. For example, if the content in the structure is accessed, the tag is altered. In some cases, the one or more features comprises fixity. In some instances, for fixity checking purposes the tag provides direct physically associated information regarding all or partial aspects of the contents of the structure (e.g., date of synthesis, subsequent handling steps, volume label and number of associated copies, etc.).

A tag may be used to identify and/or retrieve content (e.g., digital information) stored in the structure. In some instances, a tag comprises a barcode. In some examples, a barcode comprises an optical barcode. In some examples, the barcode comprises a linear barcode or a matrix (2D) barcode. In some instances, a tag comprises an electromagnetic tag. An electromagnetic tag may be identified using frequencies of the electromagnetic spectrum. Frequencies of the electromagnetic spectrum can comprise, radio frequency, microwave frequency, infrared frequency, visible frequency, UV frequency, X-ray frequency, or gamma ray frequency.

In some instances, a tag comprises a radio frequency identification (RFID) tag. In some examples, the RFID tag has a low frequency (LF), high frequency (HF) or ultra-high frequency (UHF) RFID tag. A LF RFID tag has a frequency range of about 30 KHz to 300 KHz. A HF RFID tag has a frequency range of about 3 MHz to 30 MHz. In some examples, a HF RFID tag has a frequency range of about 10 to 20 MHz. In some examples, a HF RFID tag has a frequency range of about 10, 11, 12, 13, 14, 15, 16, 17, 18, 19, or 20 MHz. In some examples, a HF RFID tag has a frequency range of at least about 10, 11, 12, 13, 14, 15, 16, 17, 18, 19, or 20 MHz. In some examples, a HF RFID tag has a frequency range of at most about 10, 11, 12, 13, 14, 15, 16, 17, 18, 19, or 20 MHz. In some examples, a HF RFID tag has a frequency range of about 13.56 MHz. A UHF RFID tag has a frequency range of about 300 MHz and 3 GHz. In some examples, an UHF RFID tag has a frequency range of about 300 MHz to 1000 MHz. In some examples, an UHF RFID tag has a frequency range of about 300 MHz to 400 MHz, 300 MHz to 500 MHz, 300 MHz to 600 MHz, 300 MHz to 700 MHz, 300 MHz to 800 MHz, 300 MHz to 900 MHz, 300 MHz to 1,000 MHz, 400 MHz to 500 MHz, 400 MHz to 600 MHz, 400 MHz to 700 MHz, 400 MHz to 800 MHz, 400 MHz to 900 MHz, 400 MHz to 1,000 MHz, 500 MHz to 600 MHz, 500 MHz to 700 MHz, 500 MHz to 800 MHz, 500 MHz to 900 MHz, 500 MHz to 1,000 MHz, 600 MHz to 700 MHz, 600 MHz to 800 MHz, 600 MHz to 900 MHz, 600 MHz to 1,000 MHz, 700 MHz to 800 MHz, 700 MHz to 900 MHz, 700 MHz to 1,000 MHz, 800 MHz to 900 MHz, 800 MHz to 1,000 MHz, or 900 MHz to 1,000 MHz. In some examples, an UHF RFID tag has a frequency range of about 300 MHz, about 400 MHz, about 500 MHz, about 600 MHz, about 700 MHz, about 800 MHz, about 900 MHz, or about 1,000 MHz. In some examples, an UHF RFID tag has a frequency range of at least about 300 MHz, about 400 MHz, about 500 MHz, about 600 MHz, about 700 MHz, about 800 MHz, or about 900 MHz. In some examples, an UHF RFID tag has a frequency range of at most about 400 MHz, about 500 MHz, about 600 MHz, about 700 MHz, about 800 MHz, about 900 MHz. or about 1,000 MHz. In some examples, an UHF RFID tag has a frequency range of about 433 MHz. In some examples, an UHF RFID tag has a frequency range of about 860 MHz to about 960 MHz. In some examples, an UHF RFID tag has a frequency range of about 860 MHz to 870 MHz, 860 MHz to 880 MHz, 860 MHz to 890 MHz, 860 MHz to 900 MHz, 860 MHz to 910 MHz, 860 MHz to 920 MHz, 860 MHz to 930 MHz, 860 MHz to 940 MHz, 860 MHz to 950 MHz, 860 MHz to 960 MHz, 870 MHz to 880 MHz, 870 MHz to 890 MHz, 870 MHz to 900 MHz, 870 MHz to 910 MHz, 870 MHz to 920 MHz, 870 MHz to 930 MHz, 870 MHz to 940 MHz, 870 MHz to 950 MHz, 870 MHz to 960 MHz, 880 MHz to 890 MHz, 880 MHz to 900 MHz, 880 MHz to 910 MHz, 880 MHz to 920 MHz, 880 MHz to 930 MHz, 880 MHz to 940 MHz, 880 MHz to 950 MHz, 880 MHz to 960 MHz, 890 MHz to 900 MHz, 890 MHz to 910 MHz, 890 MHz to 920 MHz, 890 MHz to 930 MHz, 890 MHz to 940 MHz, 890 MHz to 950 MHz, 890 MHz to 960 MHz, 900 MHz to 910 MHz, 900 MHz to 920 MHz, 900 MHz to 930 MHz, 900 MHz to 940 MHz, 900 MHz to 950 MHz, 900 MHz to 960 MHz, 910 MHz to 920 MHz, 910 MHz to 930 MHz, 910 MHz to 940 MHz, 910 MHz to 950 MHz, 910 MHz to 960 MHz, 920 MHz to 930 MHz, 920 MHz to 940 MHz, 920 MHz to 950 MHz, 920 MHz to 960 MHz, 930 MHz to 940 MHz, 930 MHz to 950 MHz, 930 MHz to 960 MHz, 940 MHz to 950 MHz, 940 MHz to 960 MHz, or 950 MHz to 960 MHz. In some examples, an UHF RFID tag has a frequency range of about 860 MHz, about 870 MHz, about 880 MHz, about 890 MHz, about 900 MHz, about 910 MHz, about 920 MHz, about 930 MHz, about 940 MHz, about 950 MHz, or about 960 MHz. In some examples, an UHF RFID tag has a frequency range of at least about 860 MHz, about 870 MHz, about 880 MHz, about 890 MHz, about 900 MHz, about 910 MHz, about 920 MHz, about 930 MHz, about 940 MHz, or about 950 MHz. In some examples, an UHF RFID tag has a frequency range of at most about 870 MHz, about 880 MHz, about 890 MHz, about 900 MHz, about 910 MHz, about 920 MHz, about 930 MHz, about 940 MHz, about 950 MHz. or about 960 MHz.

The tag can be an active tag, a passive tag or a semi-passive tag. An active tag generally refers to a tag with a transmitter and a power source. A passive tag generally refers to a tag without a power source that receives a signal from a reader with an antenna, where the tag reflects energy back to the reader. A semi-passive tag generally refers to a passive tag with a power source, which is used to turn the tag on in the presence of a signal. In some instances, the tag is an RFID tag. In some examples, the RFID tag is an active RFID tag, a passive RFID tag, or a semi-passive RFID tag. In some examples, the passive tag is a UHF RFID tag. In some examples, the passive tag is a near field communication (NFC) tag.

The tag can comprise metadata related to content stored in the structure. Metadata related to stored content of the structure (e.g., plurality of polynucleotides and/or digital information) may comprise, by way of non-limiting example, data type, data size, data format, encryption codec, date of synthesis, date of last access, dates of previous handling, owner information, manufacture information, storage mechanism, or any combination thereof. Metadata can further comprise any other information related the synthesis, storage, and/or any other handling step of the plurality of polynucleotides in the structure (e.g., volume, temperature, number of copies of the plurality of polynucleotides, etc.). In some instances, data type comprises an attribute of data (e.g., integer, Boolean, real, complex, strings, tuples, double, bytes, lists, bytes, arrays, short, long, float, character, vector, stack, sets, frozen sets, dictionaries, etc.). In some instances, a data size is the size of the digital information encoded as a plurality of polynucleotides. Data sizes can comprise, but are not limited to, sizes of items of information previously described herein. In some instances, data format comprise a format for items of information, as previously described herein. In some instances, the encryption codec can comprise an error correction code (ECC). An ECC may comprise, by way of non-limiting example, a Reed-Solomon (RS) code, a LDPC code, a polar code, a turbo code, or any combination thereof. In some instances, the date of synthesis comprises the date of synthesis of the plurality of polynucleotides. In some instances, the date of last access comprises the last date that a tag on the structure was scanned, a structure was opened, the polynucleotides were wholly or partially sequences, polynucleotides were wholly or partially decoded, or any combination thereof. In some instances, the date of previously handling comprises the dates the structure was moved or accessed, for example, by means previously described. In some instances, the owner information comprises information about the owner of the digital information, the plurality of polynucleotides, the structure, or any combination thereof. In some instances, the manufacturer information comprises information about the manufacturer of the plurality of polynucleotides, the structure, or any combination thereof. In some instances, the storage mechanism comprises a storage mechanism of the plurality of polynucleotides, such as a dehydration mechanism, ionic solvent mechanism, salt-based containment mechanism, glass-based containment mechanism, or any combination thereof.

A size of metadata can be based at least in part on the size of the structure, the format of the metadata, the size of the tag, or any combination thereof. In some instances, the size of the metadata is based at least in part on an International Organization for Standardization (ISO) standard. In some instances, the metadata is about 0.1 to 5 kB. In some instances, the metadata is about 0.1 kB to 0.5 kB, 0.1 kB to 1 kB, 0.1 kB to 1.5 kB, 0.1 kB to 2 kB, 0.1 kB to 2.5 kB, 0.1 kB to 3 kB, 0.1 kB to 3.5 kB, 0.1 kB to 4 kB, 0.1 kB to 4.5 kB, 0.1 kB to 5 kB, 0.5 kB to 1 kB, 0.5 kB to 1.5 kB, 0.5 kB to 2 kB, 0.5 kB to 2.5 kB, 0.5 kB to 3 kB, 0.5 kB to 3.5 kB, 0.5 kB to 4 kB, 0.5 kB to 4.5 kB, 0.5 kB to 5 kB, 1 kB to 1.5 kB, 1 kB to 2 kB, 1 kB to 2.5 kB, 1 kB to 3 kB, 1 kB to 3.5 kB, 1 kB to 4 kB, 1 kB to 4.5 kB, 1 kB to 5 kB, 1.5 kB to 2 kB, 1.5 kB to 2.5 kB, 1.5 kB to 3 kB, 1.5 kB to 3.5 kB, 1.5 kB to 4 kB, 1.5 kB to 4.5 kB, 1.5 kB to 5 kB, 2 kB to 2.5 kB, 2 kB to 3 kB, 2 kB to 3.5 kB, 2 kB to 4 kB, 2 kB to 4.5 kB, 2 kB to 5 kB, 2.5 kB to 3 kB, 2.5 kB to 3.5 kB, 2.5 kB to 4 kB, 2.5 kB to 4.5 kB, 2.5 kB to 5 kB, 3 kB to 3.5 kB, 3 kB to 4 kB, 3 kB to 4.5 kB, 3 kB to 5 kB, 3.5 kB to 4 kB, 3.5 kB to 4.5 kB, 3.5 kB to 5 kB, 4 kB to 4.5 kB, 4 kB to 5 kB, or about 4.5 kB to 5 kB. In some instances, the metadata is about 0.1 kB, about 0.5 kB, about 1 kB, about 1.5 kB, about 2 kB, about 2.5 kB, about 3 kB, about 3.5 kB, about 4 kB, about 4.5 kB, or about 5 kB. In some instances, the metadata is at least about 0.1 kB, about 0.5 kB, about 1 kB, about 1.5 kB, about 2 kB, about 2.5 kB, about 3 kB, about 3.5 kB, about 4 kB, or about 4.5 kB. In some instances, the metadata is at most about 0.5 kB, about 1 kB, about 1.5 kB, about 2 kB, about 2.5 kB, about 3 kB, about 3.5 kB, about 4 kB, about 4.5 kB, or about 5 kB.

In some instances, two or more structures can comprise the same plurality of polynucleotides. The two or more structure with the same plurality of polynucleotides can provide redundancy of the stored digital information. Each of the two or more structures can comprise an RFID tag. In some examples, each of the RFID tags comprises metadata that points to the other structure comprising the same plurality of polynucleotides. As an example, if structure A and structure B comprise the same plurality of polynucleotides, tag A of structure A comprises, as metadata, identifying information of structure B, such as an identification (ID) number or a universally unique identifier (UUID). Similarly, tag B on structure B comprises, as metadata, identifying information of structure A.

The metadata on a tag can reduce redundancy of the stored digital information. In some instances, the decrease in redundancy increases the payload (e.g., digital information) stored in the plurality of polynucleotides. In some instances, the decrease in redundancy increases the storage capacity. In some instances, the metadata on the tag points to one or more other structures comprising a plurality of polynucleotides. In some instances, the metadata on the tag provides a structure's location relative to other structures. In some instances, the metadata on the tag provides a plurality of polynucleotides in a structure's location relative to other polynucleotides in other structures. In some examples, the metadata is indicative of an order of the structures. Referring to FIG. 14, one or more items of digital information 1410 can be divided into a plurality of sub-items 1420. The plurality of subitems 1420 can be encoded as a plurality of polynucleotides, where each of the plurality of polynucleotides are stored in a corresponding structures 1430. In such an example, the metadata on each of the structures 1430 can comprise the order of the structures (e.g., 1, 2, 3, 4, etc.), which can be used to reconstruct the one or more items of information.

Structures in a data storage system can be indexed using a tag. In some instances, a method for storing digital information comprises one or more of: (a) synthesizing a plurality of polynucleotides, wherein the plurality of polynucleotides collectively encode digital information; (b) writing metadata relating to the plurality of polynucleotides in a tag; and (c) storing the plurality of polynucleotides in a structure. In some instances, the tag is an RFID tag. In some instances, the structure comprises the RFID tag. In some examples, the plurality of polynucleotides are synthesized in (a) and metadata relating to the plurality of polynucleotides are written in (b) simultaneously. In some examples, the plurality of polynucleotides are synthesized in (a) and metadata relating to the plurality of polynucleotides are written in (b) sequentially. In some examples, one or more of (a). (b), and (c) are performed autonomously.

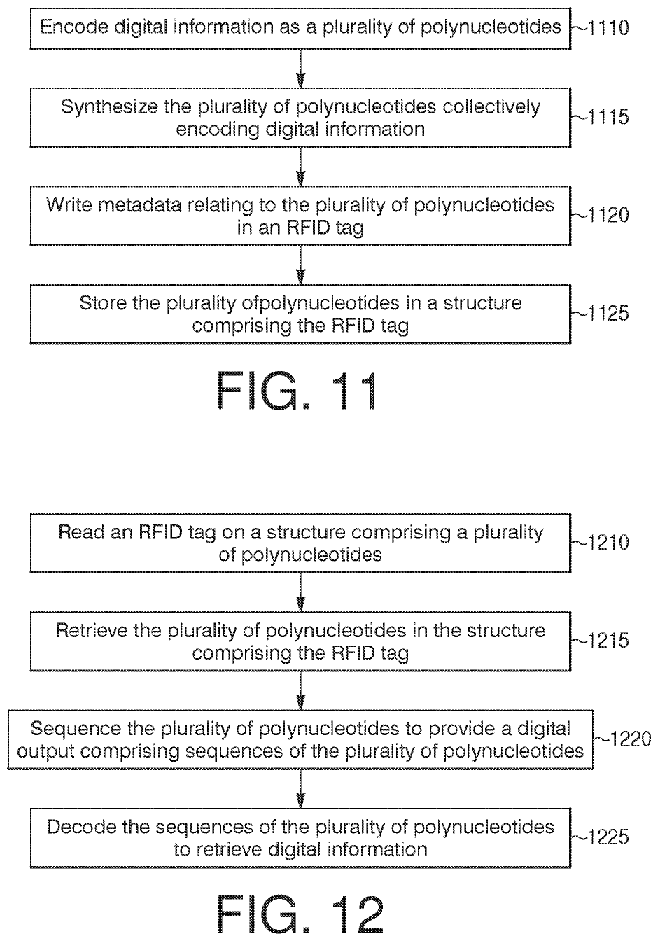

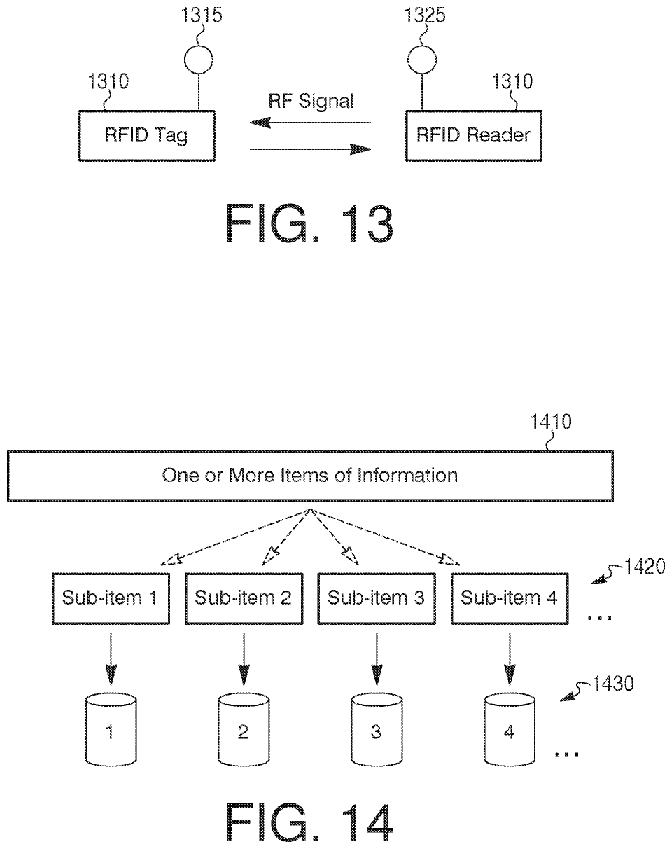

An exemplary method for storing digital information in a plurality of polynucleotides using a tag for content indexing is provided in FIG. 11. Digital information, such as an item of information described herein, can be encoded as a plurality of polynucleotides 1110. In some instances, the digital information is encoded as a plurality of polynucleotides using a codec. In some examples, the codec is a high level codec, a low level codec, or a combination thereof, such as those described herein. In some examples, the codec comprises an error correction code (ECC), such as those described herein. The plurality of polynucleotides collectively encoding the digital information are then synthesized 1115 (e.g., in a synthesizer unit). In some instances, the plurality of polynucleotides are synthesized using ligation-based synthesis, enzymatic-based synthesis, or phosphoramidite chemistry-based synthesis. In some instances, the plurality of polynucleotides are synthesized using synthesis methods further provided herein. The metadata, such as those provided herein, related to the plurality of polynucleotides is written to an RFID tag 1120. In some instances, the RFID tag is an UHF RFID tag. In some instances, the RFID tag is a passive tag. The plurality of polynucleotides are then stored in a structure comprising the RFID tag 1125. In some instances, the plurality of polynucleotides are stored in a structure before the metadata is written to the RFID tag. In some instances, the plurality of polynucleotides are stored in a structure after the metadata is written to the RFID tag. The structure can be stored in a DNA data storage system. In some instances, the DNA data storage system is partially autonomous. In some instances, the DNA data storage system is fully autonomous.

An exemplary method for retrieving digital information in a plurality of polynucleotides using a tag for content indexing is provided in FIG. 12. Digital information, such as an item of information described herein, can be encoded as a plurality of polynucleotides and stored in a structure comprising a tag in a DNA data storage system. In some instances, the tag is an RFID tag. The RFID tag can be read (e.g., scanned) by a reader 1210. In some instances, the reader scans a plurality of structures comprising an RFID tag in parallel (e.g., 100s or 1000s of structures). In some instances, the RFID tag is remotely accessed. In some instances, the structure storing the plurality of polynucleotide encoding the desired information can be identified based on metadata stored on the RFID tag. The plurality of polynucleotides are then retrieved 1215. The retrieved polynucleotides can be sequenced (e.g., in a sequencer unit) to provide a digital output comprising the sequences of the plurality of polynucleotides 1220. In some instances, the plurality of polynucleotides are amplified in an amplification chamber prior to being sequenced. The sequences of the plurality of polynucleotides are decoded to retrieve the digital information 1225. In some instances, the sequences are decode using a codec. In some examples, the codec is a high level codec, a low level codec, or a combination thereof, such as those described herein. In some examples, the codec comprises an error correction code (ECC), such as those described herein. In some instances, once the plurality of polynucleotides are accessed, the RFID tag is updated to reflect the access.

Referring to FIG. 3, an exemplary schematic of a passive RFID system is provided. In a passive RFID system, a passive RFID tag 1310 comprises an integrated circuit or chip for storing and processing information. The integrated circuit or chip can further modulate and/or demodulate a radio-frequency (RF) signal. The RFID tag can comprise a tag antenna 1315, which can receive an RF signal. Upon receiving an RF signal, an electric and magnetic field is generated. The RFID tag 1310 can draw power from the electric and magnetic field for the integrated circuit. An RFID reader 1320 comprising a reader antenna 1325 can be used to transmit the RF signal. In some instances, the RFID reader 1320 comprises a power source. The integrated circuit or chip of the RFID tag 1310 can modulate a backscatter RF signal back to the RFID reader 1320. The backscattered RF signal can comprise information (e.g., metadata) encoded in a memory of the RFID tag 1310. In some examples, the memory is a non-volatile memory. In some examples, the memory is rewritable.

An RFID reader can scan one or more RFID tags in parallel. In some instances, an RFID reader scans about 10 to 5000 RFID tags in parallel. In some instances, an RFID reader scans about 10 to 50, 10 to 100, 10 to 150, 10 to 200, 10 to 250, 10 to 500, 10 to 1,000, 10 to 1,500, 10 to 2,000, 10 to 2,500, 10 to 5.000, 50 to 100, 50 to 150, 50 to 200, 50 to 250, 50 to 500, 50 to 1,000, 50 to 1.500, 50 to 2.000, 50 to 2,500, 50 to 5,000, 100 to 150, 100 to 200, 100 to 250, 100 to 500, 100 to 1,000, 100 to 1,500, 100 to 2,000, 100 to 2,500, 100 to 5.000, 150 to 200, 150 to 250, 150 to 500, 150 to 1,000, 150 to 1.500, 150 to 2,000, 150 to 2,500, 150 to 5.000, 200 to 250, 200 to 500, 200 to 1,000, 200 to 1,500, 200 to 2,000, 200 to 2.500, 200 to 5,000, 250 to 500, 250 to 1,000, 250 to 1,500, 250 to 2.000, 250 to 2,500, 250 to 5.000, 500 to 1,000, 500 to 1,500, 500 to 2,000, 500 to 2,500, 500 to 5,000, 1,000 to 1,500, 1,000 to 2.000, 1,000 to 2.500, 1,000 to 5,000, 1,500 to 2.000, 1,500 to 2,500, 1,500 to 5,000, 2.000 to 2,500, 2,000 to 5,000, or 2,500 to 5,000 RFID tags in parallel. In some instances, an RFID reader scans about 10, 50, 100, 150, 200, 250, 500, 1000, 1500, 2000, 2500, or 5000 RFID tags in parallel. In some instances, an RFID reader scans at most about 10, 50, 100, 150, 200, 250, 500, 1000, 1500, 2000, 2500, or 5000 RFID tags in parallel. In some instances, an RFID reader scans at least about 10, 50, 100, 150, 200, 250, 500, 1000, 1500, 2000, 2500, or 5000 RFID tags in parallel.

An RFID reader can scan one or more RFID tags from a distance. In some instances, the distances is about 0.1 meters (m) to about 6 m. In some instances, the distances is 0.1 m to 0.25 m, 0.1 m to 0.5 m, 0.1 m to 0.75 m, 0.1 m to 1 m, 0.1 m to 1.5 m, 0.1 m to 2 m, 0.1 m to 2.5 m, 0.1 m to 3 m, 0.1 m to 4m, 0.1 m to 5 m, 0.1 m to 6 m, 0.25 m to 0.5 m, 0.25 m to 0.75 m, 0.25 m to 1 m, 0.25 m to 1.5 m, 0.25 m to 2 m, 0.25 m to 2.5 m, 0.25 m to 3 m, 0.25 m to 4 m, 0.25 m to 5 m, 0.25 m to 6 m, 0.5 m to 0.75 m, 0.5 m to 1 m, 0.5 m to 1.5 m, 0.5 m to 2 m, 0.5 m to 2.5 m, 0.5 m to 3 m, 0.5 m to 4 m, 0.5 m to 5 m, 0.5 m to 6 m, 0.75 m to 1 m, 0.75 m to 1.5 m, 0.75 m to 2 m, 0.75 m to 2.5 m, 0.75 m to 3 m, 0.75 m to 4m, 0.75 m to 5 m, 0.75 m to 6 m, 1 m to 1.5 m, 1 m to 2 m, 1 m to 2.5 m, 1 m to 3 m, 1 m to 4m, 1 m to 5 m, 1 m to 6m, 1.5 m to 2 m, 1.5 m to 2.5 m, 1.5 m to 3 m, 1.5 m to 4 m, 1.5 m to 5 m, 1.5 m to 6 m, 2 m to 2.5 m, 2 m to 3 m, 2 m to 4 m, 2 m to 5 m, 2 m to 6 m, 2.5 m to 3 m, 2.5 m to 4 m, 2.5 m to 5 m, 2.5 m to 6 m, 3 m to 4 m, 3 m to 5 m, 3 m to 6 m, 4 m to 5 m, 4 m to 6 m, or 5 m to 6 m. In some instances, the distances is 0.1 m, 0.25 m, 0.5 m, 0.75 m, 1 m, 1.5 m, 2 m, 2.5 m, 3 m, 4 m, 5 m, or 6 m. In some instances, the distances is at least 0.1 m, 0.25 m, 0.5 m, 0.75 m, 1 m, 1.5 m, 2 m, 2.5 m, 3 m, 4 m, or 5 m. In some instances, the distances is at most 0.25 m, 0.5 m, 0.75 m, 1 m, 1.5 m, 2 m, 2.5 m, 3 m, 4 m, 5 m, or 6 m.

Methods for content indexing described herein can be used to determine data integrity of stored digital information. The digital information can be stored in a structure comprising a tag as described herein. A method for determining data integrity of stored digital information can comprise one or more of: (a) writing metadata relating to the plurality of polynucleotides to a RFID tag, and (b) scanning the RFID tag after a duration of time to determine data integrity. In some instances, the method further comprises one or more of: (c) providing a plurality of polynucleotides collectively encoding for digital information, and (d) storing the plurality of polynucleotides in a structure. In some instances, the structure comprises an RFID tag.

An RFID tag can be valid or invalid when scanned. In some instances, a valid scan of an RFID tag comprises no errors. With no errors, a backscatter RF signal is transmitted to the RFID reader from the RFID tag. The backscatter RF signal can comprise metadata encoded in a memory of the RFID tag. The metadata can be related to the plurality of polynucleotides stored in the structure comprising the RFID tag. In some instances, a valid scan of a RFID tag is verified based at least in part on the metadata received by the reader. In some examples, the metadata encoded in an RFID tag is verified against information in a database or a file system. In some examples, the RFID tag comprises a trusted platform module (TPM). The TPM may be used to ensure integrity of the RFID tag, such as, for example, the systems and methods provided in Mubarak et al., Mutual Attestation Using TPM for Trusted RFID Protocol, Second International Conference on Network Applications, Protocols and Services, 2010. pp. 153-158. In some instances, an invalid scan of an RFID tag comprises errors. In some examples, the RFID tag is invalid if the RFID tag is not scannable or readable. An RFID tag may not be scannable if the RFID tag and/or the structure comprising the RFID tag is damaged. Non-limiting examples of an invalid RFID tag may comprise exposure of the structure to corrosive material, crushing, water, heat, humidity, or any combination thereof. In some examples, the RFID tag is invalid if the structure has been accessed without authorization and/or tampered with. In some examples, the connection between an RFID tag and an antenna is broken if the structure has been tampered with.

The RFID tag may be updated when the structure is accessed. In some examples, the RFID tag is updated (or invalidated) every time the structure is accessed. The RFID tag may be updated during one or more steps of handling of the plurality of polynucleotides. The one or more handling steps can comprise, by way of non-limiting example, synthesis, storage, sequencing, amplification, or any combination thereof. The RFID tag may be updated with metadata, such as the date, time, location, and/or any procedure information associated with the one or more handling steps. Updating the RFID tag during access of the structure can provide for data provenance, fixity, security, or any combination thereof. If the data is duplicated (e.g., during amplification) and stored in a new capsule, then a new identifying RFID tag can be written. This new RFID tag can uniquely identify the content. In some cases, the new RFID tag comprises metadata relating to one or more process steps (e.g., date, time, temperature, duration, etc. of amplification). In some instances, the new RFID tag comprises metadata relating to the one or more ‘parent’ structures. In some instances, the original RFID tag and the new RFID tag have wholly or partially the same metadata. In some instances, the original RFID tag and the new RFID tag have different metadata.

In some instances, the duration of time between after which the RFID tag is scanned is about 1 year to about 100 years. In some instances, the duration of time is 1 year to 2 years, 1 year to 5 years, 1 year to 10 years, 1 year to 20 years, 1 year to 30 years, 1 year to 40 years, 1 year to 50 years, 1 year to 60 years, 1 year to 70 years, 1 year to 80 years, 1 year to 90 years, 1 year to 100 years, 2 years to 5 years, 2 years to 10 years, 2 years to 20 years, 2 years to 30 years, 2 years to 40 years, 2 years to 50 years, 2 years to 60 years, 2 years to 70 years, 2 years to 80 years, 2 years to 90 years, 2 years to 100 years, 5 years to 10 years, 5 years to 20 years, 5 years to 30 years, 5 years to 40 years, 5 years to 50 years, 5 years to 60 years, 5 years to 70 years, 5 years to 80 years, 5 years to 90 years, 5 years to 100 years, 10 years to 20 years, 10 years to 30 years, 10 years to 40 years, 10 years to 50 years, 10 years to 60 years, 10 years to 70 years, 10 years to 80 years, 10 years to 90 years, 10 years to 100 years, 20 years to 30 years, 20 years to 40 years, 20 years to 50 years, 20 years to 60 years, 20 years to 70 years, 20 years to 80 years, 20 years to 90 years, 20 to 90 years, 30 years to 40 years, 30 years to 50 years, 30 years to 60 years, 30 years to 70 years, 30 years to 80 years, 30 years to 90 years, 30 to 100 years, 40 years to 50 years, 40 years to 60 years, 40 years to 70 years, 40 years to 80 years, 40 years to 90 years, 40 years to 100 years, 50 years to 60 years, 50 years to 70 years, 50 years to 80 years, 50 years to 90 years, 50 years to 100 years, 60 years to 70 years, 60 years to 80 years, 60 years to 90 years, 60 years to 100 years, 70 years to 80 years, 70 years to 90 years, 70 years to 100 years, 80 years to 90 years, 80 years to 100 years, or 90 to 100 years. In some instances, the duration of time is 1 year, 2 years, 5 years, 10 years, 20 years, 30 years, 40 years, 50 years, 60 years, 70 years, 80 years, 90 years, or 100 years. In some instances, the duration of time is at least 1 year, 2 years, 5 years, 10 years, 20 years, 30 years, 40 years, 50 years, 60 years, 70 years, 80 years, or 90 years. In some instances, the duration of time is at most 2 years, 5 years, 10 years, 20 years, 30 years, 40 years, 50 years, 60 years, 70 years, 80 years, 90 years, or 100 years.

During such storage periods, a minimal amount of digital information stored in polynucleotides is lost (e.g., unable to be recovered after retrieval, sequencing, and decoding) using the method, systems, and devices described herein. The amount lost includes loss of a base, loss of a series of bases, or loss of a digital information unit (e.g., bit, byte, trit, or other unit.) For example, less than 0.1 percent, or less than 0.5 percent, 1 percent, 2 percent, or less than 5 percent of the digital information is lost after 5 years of storage at 25 degrees C. In some instances, less than 0.1 percent, or less than 0.5 percent, 1 percent, 2 percent, or less than 5 percent of the digital information is lost after 5 years of storage at 30 degrees C. In some instances, less than 0.1 percent, or less than 0.5 percent, 1 percent, 2 percent, or less than 5 percent of the digital information is lost after 5 years of storage at 35 degrees C.

### Post-Processing

One or more components such as flow cells, chambers, or other component may be used for post-processing operations. In some instances, post-processing comprises addition of adapters, purification, or other step occurring after synthesis and extraction. Post processing operations in some instances occur after storage. The system for storing polynucleotides may further comprise an amplification chamber 1820. The amplification unit may be used to amplify the plurality of polynucleotides. In some instances, the system comprises more than one amplification chamber 1820. In some instances, a structure is selected from a storage unit 1815 and the polynucleotides from the structure are transferred to the amplification chamber 1820. In some instances, the polynucleotides from a synthesizer unit 1810 are transferred to the amplification chamber 1820 for size selection, PCR, or other type of amplification or preparation for storage. Size selection generally involves selecting DNA in the target size and rejecting strands that are much shorter or much longer. In some instances, filters are tuned to capture DNA of a particular size range. In some instances, other methods include PCR, electrophoresis, capture by solid phase bound primers, which are complementary to the end sequences of synthesized oligonucleotides, or the use of an isothermal polymerase. The fluidic and/or electronic control of polynucleotide synthesis in the amplification chamber 1820 may be performed by a controller 1835. In some instances, the electronics in the amplification chamber 1820 are in communication with the controller 1835. In some instances, post-processing modules and operations are shown in FIG. 2B. In some instances, a module for amplification comprises one or more of a thermal cycler, a seal, and a chamber for rehydration and aliquoting.

### Encoding

Provided herein are devices, assemblies, compositions, systems and methods for nucleic acid-based information (data) storage. In some instances, the devices, compositions, platforms, systems, or methods provided herein are used for DNA data storage of biomolecules that have been extracted from a substrate. In some instances, these devices, assemblies, compositions, systems and methods are used for encapsulation of polynucleotides encoding digital information for DNA data storage. In some examples, the system comprises a base plate (and/or cooling plate), a plurality of compartments, and a cover plate. The plurality of compartments and/or base plate can comprise a material with a smaller coefficient of thermal expansion (CTE) than the material of the cover plate, such that the cover plate (which may be heated prior) can be placed on the compartments, and subsequently cooled to seal the compartments. In some further instances, these devices, compositions, systems and methods are used for DNA data storage using a radio-frequency identification (RFID) tag. A biomolecule such as a DNA molecule provides a suitable host for storage of information, such as digital information, in-part due to its stability over time and capacity for enhanced information coding, as opposed to traditional binary information coding. In addition, a biomolecule such as a DNA molecule can provide high volumetric storage density. In a first step, a digital sequence encoding an item of information (e.g., digital information in a binary code for processing by a computer) is received. The digital sequence can comprise a first plurality of symbols, such a binary, octal, decimal, or hexadecimal data. An encryption scheme is applied to convert the digital sequence from the first string of symbols to a second string of symbols. The second string of symbols can comprise an alternative representation to the first string of symbols. In some examples, the second string of symbols comprises a nucleic acid sequence.

Once an item of information is converted to a nucleic acid sequence, the nucleic acids can be synthesized. A surface material for nucleic acid extension, a design for loci for nucleic acid extension (aka, arrangement spots), and reagents for nucleic acid synthesis are selected. The surface of a structure is prepared for nucleic acid synthesis. De novo polynucleotide synthesis is then performed. The synthesized polynucleotides can be extracted, in whole or in part, using the systems, devices, methods, or platforms provided herein. The synthesized polynucleotides are stored in a structure and, in some cases, are available for subsequent release, in whole or in part. The synthesized polynucleotides may be stored in a structure suitable for long term storage (e.g., weeks, months, years, etc.). A structure suitable for long term storage may be identifiable and/or capable of being catalogues, such as, for example, using a tag (e.g., barcode or RFID tag). Once released, the polynucleotides, in whole or in part, are sequenced, subject to decryption to convert nucleic sequence back to digital sequence. The digital sequence is then assembled to obtain an alignment encoding for the original item of information.

### Items of Information

Optionally, an early step of data storage process disclosed herein includes obtaining or receiving one or more items of information in the form of an initial code. In some instances, the items of information are encoded as a plurality of polynucleotides that have been extracted from a substrate, using systems, methods, platforms, or devices provided herein. In some instances, the items of information are encoded as a plurality of polynucleotides and encapsulated using the systems and methods described herein. In some examples, the system comprises a base plate (and/or cooling plate), a plurality of compartments, and a cover plate. The plurality of compartments and/or base plate can comprise a material with a smaller coefficient of thermal expansion (CTE) than the material of the cover plate, such that the cover plate (which may be heated prior) can be placed on the compartments, and subsequently cooled to seal the compartments. In some further instances, the items of information are encoded as a plurality of polynucleotides and stored in a structure comprising a radio-frequency identification (RFID) tag. Items of information (e.g., digital information) include, without limitation, text, audio and visual information. Exemplary sources for items of information include, without limitation, books, periodicals, electronic databases, medical records, letters, forms, voice recordings, animal recordings, biological profiles, broadcasts, films, short videos, emails, bookkeeping phone logs, internet activity logs, drawings, paintings, prints, photographs, pixelated graphics, and software code. Exemplary biological profile sources for items of information include, without limitation, gene libraries, genomes, gene expression data, and protein activity data. Exemplary formats for items of information include, without limitation, .txt, .PDF, .doc, .docx, .ppt, .pptx, .xls, .xlsx, .rtf, .jpg, .gif, .psd, .bmp, tiff, .png, and, mpeg. The amount of individual file sizes encoding for an item of information, or a plurality of files encoding for items of information, in digital format include, without limitation, up to 1024 bytes (equal to 1 KB), 1024 KB (equal to 1 MB), 1024 MB (equal to 1 GB), 1024 GB (equal to 1 TB), 1024 TB (equal to 1 PB), 1 exabyte, 1 zettabyte, 1 yottabyte, 1 xenottabyte or more. In some instances, an amount of digital information is at least 1 gigabyte (GB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 gigabytes. In some instances, the amount of digital information is at least 1 terabyte (TB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 terabytes. In some instances, the amount of digital information is at least 1 petabyte (PB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 petabytes. In some instances, the digital information does not contain genomic data acquired from an organism. Items of information in some instances are encoded. Non-limiting encoding method examples include 1 bit/base, 2 bit/base, 4 bit/base or other encoding method.

### Sequencing

Polynucleotides are extracted and/or amplified from surfaces where they are synthesized or stored. After extraction and/or amplification of polynucleotides from the surface of a structure, suitable sequencing technology may be employed to sequence the polynucleotides. In some cases, the DNA sequence is read on the substrate or within a feature of a structure. In some cases, the polynucleotides stored on the substrate are extracted is optionally assembled into longer polynucleotides and then sequenced.

### Sequencing/Decoding

The system for storing polynucleotides may further comprise a sequencing unit 1825. The sequencing unit 1825 may be used to sequence a plurality of polynucleotides. In some instances, the plurality of polynucleotides are transferred from the amplification chamber 1820 to the sequencing unit 1825. In some instances, the system may comprise additional modules for performing additional sequencing preparation steps. In some examples, the plurality of polynucleotides are transferred from the amplification chamber 1820 to the sequencing unit 1825 using one or more tubes or the robotic system 1830. In some instances, the amplification chamber 1820 and the sequencing unit 1825 are fluidically coupled. The fluidic and/or electronic control of polynucleotide synthesis in the sequencing unit 1825 may be performed by a controller 1835. In some instances, the electronics in the sequencing unit 1825 are in communication with the controller 1835.

In some instances, the system comprises large-scale sequencing of polynucleotides. In some instances, large-scale sequencing comprises dense and highly parallel sequencers. In some instances, the system comprises more than one sequencing unit 1825. In some instances, the sequencing unit 1825 use centrifugal forces and/or vacuum/pressure to add or evacuate reagents from the sequencing unit 1825. In some instances, the sequencing unit 1825 is light-based (e.g., with light sources and sensors on chip), nanopore-based (e.g., Oxford Nanopore Technologies (ONT)), or involve other operations (e.g., a light-based method such as PacBio/SMRT single-molecule smart sequencing or other sequencing technologies). In some instances, the sequencing unit 1825 employs sequencing methods provided herein. In some instances, the sequencing unit 1825 uses of nanopores or other electrical sequencing technology that benefits from the bulk fluidics provided by semiconductor fabrication equipment. In some instances, the one or more modules described herein comprises a camera. A camera may be used to capture one or more optical features of polynucleotides in a module. As an example, a camera may be used in a synthesizer unit, a sequencing unit, or both, to capture an optical feature of polynucleotides attached to a surface on a solid support as described herein. In some instances sequencing comprises use of nanoball generation (e.g., MGI/BGI) and sequencing by synthesis. In some instances sequencing comprises sequencing by synthesis (e.g., Illumina).

The system for storing polynucleotides can comprise a robotic system 1830 as described herein. The robotic system may generally be used to manipulate the polynucleotides in a system. Manipulation can comprise, without limitation, moving, storing, retrieving, handling, transferring, or any combination thereof. In some instances, the robotic system transfers the plurality of polynucleotides between modules in the system. In some examples, the robotic system manipulates (e.g., transfers) the plurality of polynucleotides in structure for storage as described herein. In some instances, the robotic system manipulates (e.g., transfers) the plurality of polynucleotides in a rack. In some examples, the rack comprises a plurality of structures each comprising an RFID tag. In some examples, the rack comprises a plurality of solid supports for synthesis and/or sequencing. In some instances, the robotic system comprises a robotic hand or a robotic picker. In some instances, the robotic system 1830 is fully integrated with the storage system control software and/or firmware in the controller 1835. In some instances, the robotic system 1830 is fully integrated with an external host application. In some instances, the robotic system 1830 is fully automated.

The system for storing polynucleotides can comprise a controller 1835. The controller may generally be used for controlling modules, components, fluidics, robots, or any combination thereof. The modules, components, fluidics, electronics, robots, or any combination thereof may be used for synthesizing, storing, retrieving, sequencing, and/or amplifying polynucleotides. In some instances, the controller 1835 is capable of cataloguing all storage structures loaded, unloaded, and/or stored within a rack. The polynucleotides can encode digital information as described herein. The modules, components, fluidics, electronics, robots, or any combination thereof may be used for performing methods, models, or algorithms, such as encoding or decoding the polynucleotides.

In some instances, the controller 1835 controls the physical location of the plurality of polynucleotides. In some instances, the controller 1835 provides commands to one or more modulus of the system. In some examples, the controller 1835 controls robotics (e.g., robotic system 1830), actuators, and fluidic valves, or any other equipment of the system. In some instances, the controller 1835 allows for synchronizing and controlling the modules for processing and/or transferring polynucleotides. In some examples, the polynucleotides are processed and/or transferred via fluidics. In some examples, the controller 1835 controls one or more valves or parameters (e.g., pressure, vacuum, temperature, volume, etc.) in the system for biomolecule extraction, for example, as provided in FIG. 16. In some examples, the controller 1835 can be used to orient or adjust the orientation of a flow cell in a system for biomolecule synthesis and/or extraction. This can allow for flexibility of the system and maximize recovery of liquid or material (e.g., polynucleotides). In some examples, the polynucleotides are processed and/or transferred via electronics. In some instances, the controller 1835 controls physical parameters in one or more modules, such as, without limitation, pressure, vacuum, temperature, volume (e.g., of fluids), or any combination thereof.

In some instances, the controller 1835 invokes an encoder module or a decoder module. In some instances, the encoder module encodes the digital information as a plurality of polynucleotides. In some instances, the encoder module applies one or more codecs, such as those described herein, to the digital information. In some instances, the decoder module decodes the sequences of the plurality of polynucleotides to retrieve the digital information. In some instances, the decoder module applies one or more codecs, such as those described herein, to the sequences of the plurality of polynucleotides. In some instances, the decode module performs reassembly, error correction, and outputs digital information (e.g., binary data). In some instances, the output comprising digital information is transferred to an operating system and/or a file system. The output may be provided on a display, such as a graphical user interface (GUI), or any other suitable display such as those described herein, for providing the digital information. In some instances, the controller 1835 is implemented on one or more software modules, such as those described herein. In some instances, the controller 1835 responds to commands from an operating system, such as those described herein.

An encoder module generally encodes the digital information as a plurality of polynucleotides. An encoder can apply an encoding scheme to digital information. In some instances, the encoding scheme comprises codecs for encoding binary data as polynucleotide sequences (e.g., inner codec). In some instances, the encoding scheme comprises an error correction code (ECC) (e.g., outer codec). In some instances, employing a flow cell optimized for maximum recovery of material from a substrate decreases the need for error correction, since less material is lost between transfers of material. In some cases, the encoding scheme is designed and implemented to allow streaming read and write API access. In some cases, the encoding scheme is designed and implemented to match the streaming of the systems and methods for digital storage described herein.

The encoding scheme can generally comprise one or more operations. The one or more operations can comprise one or more operation to manipulate or transform data (e.g., digital information). The one or more operations can comprise by way of non-limiting example, splitting, shuffling, concatenating, transposing, translating, duplicating, labeling (e.g., using an index) data or a part of the data, or any combination thereof.

In some instances, the outer codec comprises an error correction code (ECC) or scheme, such as, without limitation, a Reed-Solomon (RS) code, a low-density parity-check (LDPC) code, a polar code, a turbo code, or any variation thereof. This outer codec is used for spreading the digital or binary data to be stored over many oligonucleotides. In some instances, spreading the data builds redundancy to correct for erasures (e.g., lost oligonucleotides). In some further embodiments, spreading the data also builds redundancy to correct errors from an inner codec. In some instances, the methods for encoding digital or binary data in a plurality of nucleotide sequences comprise an inner codec. In some instances, the inner codec is applied to the binary data. In some instances, the inner codec is applied to the binary data from the ECC. In some instances, the inner codec is applied to the lanes of the binary data. In some instances, the inner codec is applied to binary data that has been shuffled.

In some instances, the encoding scheme comprises an inner codec. In some instances, an inner codec is applied to encode the binary data as a polynucleotide sequence. The inner codec is used to transform digital or binary data into nucleotide bases. In some instances, the inner codec is capable of correcting deletion, substitution, or insertion errors, or any combination thereof. In some further embodiments, the inner codec is used to validate oligonucleotides and discard oligonucleotides which fail error checking algorithms to avoid contaminating the outer decoding. The inner codec further encodes the indices, which can allow for efficient clustering during decoding. In some instances, the encoding scheme adds redundancy across the plurality of oligonucleotide sequences. In some instances, the inner codec comprises generating base candidates. In some instances, base candidates are generated using a codebook, a lookup table, a hash, or any suitable method known in the art. In some instances, the inner codec further comprises a base repetition check. In some instances, the inner codec further comprises performing GC filtering.

A decoder module generally decodes the sequences of the plurality of polynucleotides to retrieve the digital information. A decoder can apply a decoding scheme to the sequences of the plurality of polynucleotides. In some instances, a decoding scheme comprises an inner codec, an outer codec (e.g., ECC), or a combination thereof. In some instances, the decoding scheme decodes a plurality of polynucleotide sequences to generate an output comprising digital information. In some instances, the decoding scheme comprises undoing operations in the encoding scheme. In some examples, the operations comprise, without limitation, splitting, shuffling, concatenating, transposing, translating, duplicating, labeling (e.g., using an index) data or a part of the data, or any combination thereof.

A digital output from a sequencer unit comprising sequences of the plurality of polynucleotides may be provided to the decoding module. In some instances, the decoder module orders, clusters, and/or aligns sequences of the plurality of polynucleotides. In some examples, the decoder module comprises an alignment algorithm, such as with limitation, a pairwise alignment algorithm, a multi-sequence alignment algorithm, or any other suitable algorithm.

In some instances, decoding scheme comprise an inner codec. In some instances, the inner codec is applied to the plurality of polynucleotide sequences. The inner codec is used to transform the polynucleotide sequences into digital or binary data. In some instances, the inner codec is capable of correcting deletion, substitution, or insertion errors, or any combination thereof. In some further embodiments, the inner codec is used to validate oligonucleotides and discard any suspicious oligonucleotides to avoid contaminating the outer decoding. In some instances, the inner codec allows for efficient decoding using the indices.

An inner codec comprising a decoding scheme can be applied to the plurality of polynucleotide sequences. In some instances, the inner codec transforms each of the plurality of polynucleotide sequences into binary data. In some instances, the inner codec is applied to a plurality of polynucleotides that have been sequenced. In some examples, the plurality of clustered have been ordered, clustered, aligned, or any combination thereof. In some instances, one or more codecs comprises a cyclic redundancy check (CRC).

In some instances, the inner codec comprises a greedy algorithm. A greedy algorithm generally takes into account transitions from only the most probably state as it decodes each bit position in a sequence. In some instances, the inner codec comprises a maximum likelihood (ML) algorithm. A ML algorithm generally takes into account transitions from all states as it decodes each bit position in a sequence In some instances, the inner codec comprises a mixed greedy ML algorithm. A mixed greedy ML algorithm can generally take into account transitions from a plurality of states as it decodes each bit position in a sequence. In some instances, the inner codec comprises a beam search decoder or a random sampling decoder (e.g., pure sampling decoder, a top-K sampling decoder, etc.). In some cases, a beam search decoder or a random sampling decoder provides a diversity of candidate states compared to a greedy decoder. In some instances, the inner codec further comprises a checksum. In some instances, the inner codec comprises a hash (e.g., SHA-256). In some instances, the hash verifies that the data was correctly decoded. In some instances, by using a hash at the end (after the ECC), the encoding and decoding are performed as a stream. In some instances, this can limit memory use to only temporary buffers.

In some cases, an inner codec is applied to the subset of the plurality of data polynucleotides. In some instances, the inner codec comprises probabilistic decoding. An inner codec generally comprises a decoding polynucleotides into digital information. In some cases, the inner codec comprises converting or transforming each of the subset of the plurality of data polynucleotides into binary data. In some instances, a full length of the subset of the plurality of data polynucleotides are transformed or converted into binary data (e.g., full decoding). In some instances, a partial length of the subset of the plurality of data polynucleotides are transformed or converted into binary data (e.g., partial decoding). In some examples, the partial length comprises an index, such as those described herein (e.g., lane index, frame index. UUID, content ID, etc.). In some instances, the inner codec is applied to the subset of the plurality of data polynucleotides that have been sequenced. In some instances, the inner codec is applied to the subset of the plurality of data polynucleotides that have or have not been ordered, aligned, clustered, or any combination thereof.

In some cases, the plurality of data polynucleotides and/or the subset of the plurality of data polynucleotides are encoded using the methods described herein. In some cases, the plurality of data polynucleotides and/or the subset of the plurality of data polynucleotides are decoded using the methods described herein. In some instances, the inner codec comprises a greedy algorithm. In some instances, the inner codec comprises a maximum likelihood (ML) algorithm. In some instances, the inner codec comprises a mixed greedy ML algorithm.

In some cases, the probabilistic decoding of the inner codec provides a likelihood of the overall decoded sequence. In some instances, redundancy within each polynucleotide sequences helps to estimate error rates without knowing a reference polynucleotide. As an example, if the inner codec decodes sequences with high probabilities and/or in very few steps, then the error rate is likely low. As a further example, if the inner codec decodes sequences with low probabilities and/or takes more steps, then the error rate is likely high.

In some cases, the data polynucleotides comprises an index. In some instances, an index of the subset of the plurality of data polynucleotides is decoded. In some instances, the index is decoded using an inner codec, an outer codec, or a combination thereof, such as, but not limited to, those described herein. In some instances, the index is used to estimate a relative distribution of the subset of the plurality of polynucleotides. In some examples, the relative distribution is used to estimate uniformity of the data polynucleotides. For example, if the plurality of data polynucleotides comprises about 100.000 polynucleotide sequences, and the subset selected is 0.1% of the data polynucleotides, a distribution centered around 100 decoded indexes can be expected. In some examples, relative distribution changes between subsets of the data polynucleotides indicate a loss of uniformity across the data polynucleotides.

In some instances, the decoding module comprises an outer codec (e.g., ECC). In some instances, the plurality of nucleotide sequences are decoded into digital or binary data. In some instances, an outer codec (e.g., ECC) is applied to the digital or binary data. In some instances, the outer codec comprises an ECC used to encode the data (e.g., binary data). In some instances, the ECC comprises a Reed-Solomon (RS) code, a LDPC code, a polar code, a turbo code, or any combination thereof. In some instances, the decoding scheme comprises soft decoding. Soft decoding generally refers to decoding by considering a range of possible values (e.g., using probability estimates).

Polynucleotides synthesized and stored on the structures described herein encode data that can be interpreted by reading the sequence of the synthesized polynucleotides and converting the sequence into binary code readable by a computer. In some cases the sequences require assembly, and the assembly step may need to be at the nucleic acid sequence stage or at the digital sequence stage. Polynucleotides are extracted and/or amplified from surfaces where they are synthesized or stored. After extraction and/or amplification of polynucleotides from the surface of a structure, suitable sequencing technology may be employed to sequence the polynucleotides. In some cases, the DNA sequence is read on the substrate or within a feature of a structure. In some cases, the polynucleotides stored on the substrate are extracted is optionally assembled into longer polynucleotides and then sequenced.

Provided herein are detection systems comprising a device capable of sequencing stored polynucleotides, either directly on the synthesis structure and/or after removal from the main structure (e.g., synthesis structure, storage structure, etc.). In cases where the synthesis structure is a reel-to-reel tape of flexible material, the detection system comprises a device for holding and advancing the structure through a detection location and a detector disposed proximate the detection location for detecting a signal originated from a section of the tape when the section is at the detection location. In some instances, the signal is indicative of a presence of a polynucleotide. In some instances, the signal is indicative of a sequence of a polynucleotide (e.g., a fluorescent signal). In some instances, information encoded within polynucleotides on a continuous tape is read by a computer as the tape is conveyed continuously through a detector operably connected to the computer. In some instances, a detection system comprises a computer system comprising a polynucleotide sequencing device, a database for storage and retrieval of data relating to polynucleotide sequence, software for converting DNA code of a polynucleotide sequence to binary code, a computer for reading the binary code, or any combination thereof.

Provided herein are sequencing systems that can be integrated into the devices described herein. Various methods of sequencing are well known in the art, and comprise “base calling” wherein the identity of a base in the target polynucleotide is identified. In some instances, polynucleotides synthesized using the methods, devices, compositions, and systems described herein are sequenced after cleavage from the synthesis surface. In some instances, sequencing occurs during or simultaneously with polynucleotide synthesis, wherein base calling occurs immediately after or before extension of a nucleoside monomer into the growing polynucleotide chain. Methods for base calling include measurement of electrical currents/voltages generated by polymerase-catalyzed addition of bases to a template strand. In some instances, synthesis surfaces comprise enzymes, such as polymerases. In some instances, such enzymes are tethered to electrodes or to the synthesis surface. In some instances, enzymes comprise terminal deoxynucleotidyl transferases, or variants thereof.

### Commuting System

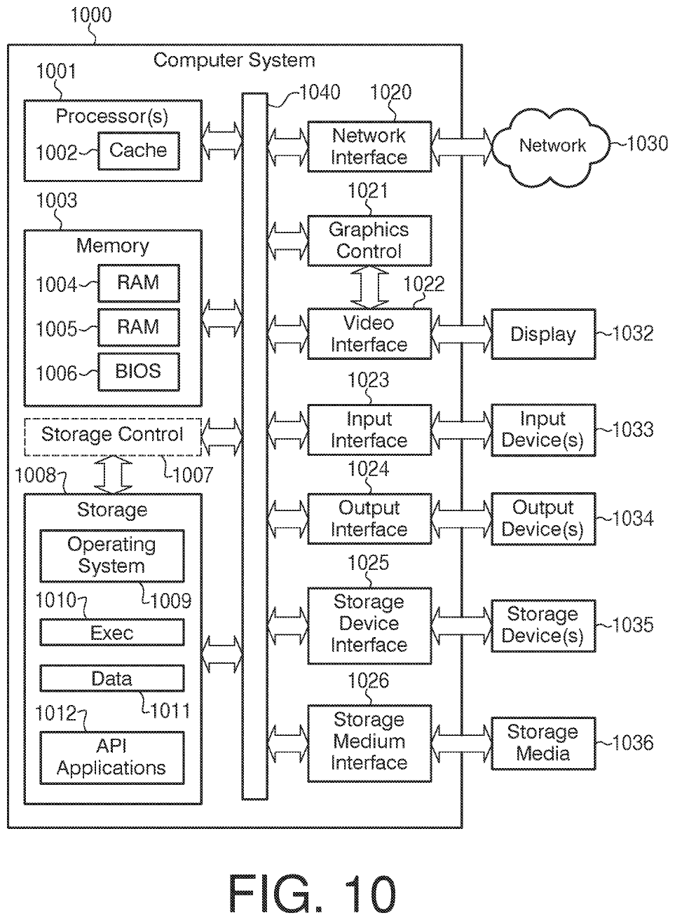

Referring to FIG. 10, a block diagram is shown depicting an exemplary machine that includes a computer system 1000 (e.g., a processing or computing system) within which a set of instructions can execute for causing a device to perform or execute any one or more of the aspects and/or methodologies for static code scheduling of the present disclosure. The components in FIG. 10 are examples only and do not limit the scope of use or functionality of any hardware, software, embedded logic component, or a combination of two or more such components implementing particular embodiments. A computing system as generally illustrated in FIG. 10 may be part of a data storage system, as exemplary illustrated in FIG. 8.

In various aspects, any of the systems (e.g., FIGS. 1-8) described herein are operably linked to a computer and are optionally automated through a computer either locally or remotely. In various instances, the methods and systems described herein further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the disclosure provided herein. In some instances, the computer systems are programmed to interface between the user specified base sequence and the position of a material deposition device to deliver the correct building blocks and/or reagents to specified regions of the substrate (e.g., specific loci).

As an example, a computer system, such as the system shown in FIG. 10, may be used for encoding data represented as a set of symbols to another set of symbols. For example, the data may be represented as numerical symbols, such as binary values of “0”s and “1” s and the computer system may execute a program comprising a codec (e.g., an error correction code, such as RS code, LDPC code, Turbo code, etc.). In some instances, the computer system converts a first string of symbols to a second string of symbols using the program. In some instances, the computer system executes a program to convert the data to a plurality of nucleic acid sequences, convert a plurality of nucleic acid sequences to data, or both. In some instances, the computer system executes a program to convert a first one or more nucleic acid sequence to a second one or more nucleic acid sequences. For example, the computer program may convert a first one or more nucleic acid sequence to a second one or more nucleic acid sequence, where the second one or more nucleic acid sequences is more resistant to oxidation compared to the first one or more nucleic acid sequences.

As an example, a computer system, such as the system shown in FIG. 10, may be used for monitoring one or more components in a data storage system (e.g., FIGS. 1-8). For example, the computer system may be used to monitor one or more sensor data from a sensor integrated in or connected to a components or modules in the systems illustrated herein. In some instances, the computer system employs a program to monitor and detect irregularities in one or more parameters, such as pressure, volume, flow rate, temperature, vacuum, angles of orientation, humidity, or any other physical parameters that can be measured in the systems and platforms described herein. The computer system comprising the program may analyze patterns in one or more sensor data and optionally alert a user through an HMI if any irregularities are detected or if any data or combination of data fall outside of a threshold (e.g., predetermined or dynamic thresholds).

A program may be executed on a computer system provided herein. In some instances, a program comprises a statistical algorithm or a machine learning algorithm. In some instances, an algorithm comprising machine learning (ML) is trained to perform the functions or operations described herein. In some cases, the algorithm comprises classical ML algorithms for classification and/or clustering (e.g., K-means clustering, mean-shift clustering, density-based spatial clustering of applications with noise (DBSCAN), expectation-maximization (EM) clustering, agglomerative hierarchical clustering, logistic regression, naïve Bayes, K-nearest neighbors, random forests or decision trees, gradient boosting, support vector machines (SVMs), or a combination thereof).

In some cases, the algorithm comprises a learning algorithm comprising layers, such as one or more neural networks. Neural networks may comprise connected nodes in a network, which may perform functions, such as transforming or translating input data. In some examples, the output from a given node may be passed on as input to another node. In some embodiments, the nodes in the network may comprise input units, hidden units, output units, or a combination thereof. In some cases, an input node may be connected to one or more hidden units. In some cases, one or more hidden units may be connected to an output unit. The nodes may take in input and may generate an output based on an activation function. In some embodiments, the input or output may be a tensor, a matrix, a vector, an array, or a scalar. In some embodiments, the activation function may be a Rectified Linear Unit (ReLU) activation function, a sigmoid activation function, or a hyperbolic tangent activation function. In some embodiments, the activation function may be a Softmax activation function. The connections between nodes may further comprise weights for adjusting input data to a given node (e.g., to activate input data or deactivate input data). In some embodiments, the weights may be learned by the neural network. In some embodiments, the neural network may be trained using gradient-based optimizations. In some cases, the gradient-based optimization may comprise of one or more loss functions. In some examples, the gradient-based optimization may be conjugate gradient descent, stochastic gradient descent, or a variation thereof (e.g., adaptive moment estimation (ADAM)). In further examples, the gradient in the gradient-based optimization may be computed using backpropagation. In some embodiments, the nodes may be organized into graphs to generate a network (e.g., graph neural networks). In some embodiments, the nodes may be organized into one or more layers to generate a network (e.g., feed forward neural networks, convolutional neural networks (CNNs), recurrent neural networks (RNNs), etc.). In some cases, the neural network may be a deep neural network comprising of more than one layer.

In some cases, the neural network may comprise one or more recurrent layer. In some examples, the one or more recurrent layer may be one or more long short-term memory (LSTM) layers or gated recurrent unit (GRU), which may perform sequential data classification and clustering. In some embodiments, the neural network may comprise one or more convolutional layers. The input and output may be a tensor representing of variables or attributes in a data set (e.g., features), which may be referred to as a feature map (or activation map). In some cases, the convolutions may be one dimensional (1D) convolutions, two dimensional (2D) convolutions, three dimensional (3D) convolutions, or any combination thereof. In further cases, the convolutions may be 1D transpose convolutions. 2D transpose convolutions, 3D transpose convolutions, or any combination thereof. In some examples, one-dimensional convolutional layers may be suited for time series data since it may classify time series through parallel convolutions. In some examples, convolutional layers may be used for analyzing a signal (e.g., sensor data) from one or more components of a system described herein.

The layers in a neural network may further comprise one or more pooling layers before or after a convolutional layer. The one or more pooling layers may reduce the dimensionality of the feature map using filters that summarize regions of a matrix. This may down sample the number of outputs, and thus reduce the parameters and computational resources needed for the neural network. In some embodiments, the one or more pooling layers may be max pooling, min pooling, average pooling, global pooling, norm pooling, or a combination thereof. Max pooling may reduce the dimensionality of the data by taking only the maximums values in the region of the matrix, which helps capture the significant feature. In some embodiments, the one or more pooling layers may be one dimensional (1D), two dimensional (2D), three dimensional (3D), or any combination thereof. The neural network may further comprise of one or more flattening layers, which may flatten the input to be passed on to the next layer. In some cases, the input may be flattened by reducing it to a one-dimensional array. The flattened inputs may be used to output a classification of an object (e.g., classification of signals (e.g., sensor data) in a system described herein). The neural networks may further comprise one or more dropout layers. Dropout layers may be used during training of the neural network (e.g., to perform binary or multi-class classifications). The one or more dropout layers may randomly set certain weights as 0, which may set corresponding elements in the feature map as 0, so the neural network may avoid overfitting. The neural network may further comprise one or more dense layers, which comprise a fully connected network. In the dense layer, information may be passed through the fully connected network to generate a predicted classification of an object, and the error may be calculated. In some embodiments, the error may be backpropagated to improve the prediction. The one or more dense layers may comprise a Softmax activation function, which may convert a vector of numbers to a vector of probabilities. These probabilities may be subsequently used in classifications, such as classifications of signal (e.g., sensor data) from a system described herein, or probable nucleobases during decoding (e.g., as part of a codec).

Computer system 1000 (FIG. 10) may include one or more processors 1001, a memory 1003, and a storage 1008 that communicate with each other, and with other components, via a bus 1040. The bus 1040 may also link a display 1032, one or more input devices 1033 (which may, for example, include a keypad, a keyboard, a mouse, a stylus, etc.), one or more output devices 1034, one or more storage devices 1035, and various tangible storage media 1036. All of these elements may interface directly or via one or more interfaces or adaptors to the bus 1040. For instance, the various tangible storage media 1036 can interface with the bus 1040 via storage medium interface 1026. Computer system 1000 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 1000 includes one or more processor(s) 1001 (e.g., central processing units (CPUs), general purpose graphics processing units (GPGPUs), or quantum processing units (QPUs)) that carry out functions. Processor(s) 1001 optionally contains a cache memory unit 1002 for temporary local storage of instructions, data, or computer addresses. Processor(s) 1001 are configured to assist in execution of computer readable instructions. Computer system 1000 may provide functionality for the components depicted in FIG. 10 as a result of the processor(s) 1001 executing non-transitory, processor-executable instructions embodied in one or more tangible computer-readable storage media, such as memory 1003, storage 1008, storage devices 1035, and/or storage medium 1036. The computer-readable media may store software that implements particular embodiments, and processor(s) 1001 may execute the software. Memory 1003 may read the software from one or more other computer-readable media (such as mass storage device(s) 1035, 1036) or from one or more other sources through a suitable interface, such as network interface 1020. The software may cause processor(s) 1001 to carry out one or more processes or one or more steps of one or more processes described or illustrated herein. Carrying out such processes or steps may include defining data structures stored in memory 1003 and modifying the data structures as directed by the software.

The memory 1003 may include various components (e.g., machine readable media) including, but not limited to, a random access memory component (e.g., RAM 1004) (e.g., static RAM (SRAM), dynamic RAM (DRAM), ferroelectric random access memory (FRAM), phase-change random access memory (PRAM), etc.), a read-only memory component (e.g., ROM 1005), and any combinations thereof. ROM 1005 may act to communicate data and instructions unidirectionally to processor(s) 1001, and RAM 1004 may act to communicate data and instructions bidirectionally with processor(s) 1001. ROM 1005 and RAM 1004 may include any suitable tangible computer-readable media described below. In one example, a basic input/output system 1006 (BIOS), including basic routines that help to transfer information between elements within computer system 1000, such as during start-up, may be stored in the memory 1003.

Fixed storage 1008 is connected bidirectionally to processor(s) 1001, optionally through storage control unit 1007. Fixed storage 1008 provides additional data storage capacity and may also include any suitable tangible computer-readable media described herein. Storage 1006 may be used to store operating system 1009, executable(s) 1010, data 1011, applications 1012 (application programs), and the like. Storage 1008 can also include an optical disk drive, a solid-state memory device (e.g., flash-based systems), or a combination of any of the above. Information in storage 1008 may, in appropriate cases, be incorporated as virtual memory in memory 1003.

In one example, storage device(s) 1035 may be removably interfaced with computer system 1000 (e.g., via an external port connector (not shown)) via a storage device interface 1025. Particularly, storage device(s) 1035 and an associated machine-readable medium may provide non-volatile and/or volatile storage of machine-readable instructions, data structures, program modules, and/or other data for the computer system 1000. In one example, software may reside, completely or partially, within a machine-readable medium on storage device(s) 1035. In another example, software may reside, completely or partially, within processor(s) 1001.

Bus 1040 connects a wide variety of subsystems. Herein, reference to a bus may encompass one or more digital signal lines serving a common function, where appropriate. Bus 1040 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus. HyperTransport (HTX) bus, serial advanced technology attachment (SATA) bus, and any combinations thereof.

Computer system 1000 may also include an input device 1033. In one example, a user of computer system 1000 may enter commands and/or other information into computer system 1000 via input device(s) 1033. Examples of an input device(s) 1033 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 1033 may be interfaced to bus 1040 via any of a variety of input interfaces 1023 (e.g., input interface 1023) including, but not limited to, serial, parallel, game port, USB, FIREWIRE. THUNDERBOLT, or any combination of the above.

In particular embodiments, when computer system 1000 is connected to network 1030, computer system 1000 may communicate with other devices, specifically mobile devices and enterprise systems, distributed computing systems, cloud storage systems, cloud computing systems, and the like, connected to network 1030. In some embodiments, the computing system 1000 may communicate with one or more components of a system of data storage (e.g., FIGS. 1-8). For example, the computing system 1000 may communicate with (e.g., control or manage) the robotic system 1330. Communications to and from computer system 1000 may be sent through network interface 1020. For example, network interface 1020 may receive incoming communications (such as requests or responses from other devices) in the form of one or more packets (such as Internet Protocol (IP) packets) from network 1030, and computer system 1000 may store the incoming communications in memory 1003 for processing. Computer system 1000 may similarly store outgoing communications (such as requests or responses to other devices) in the form of one or more packets in memory 1003 and communicated to network 1030 from network interface 1020. In some embodiments, the computing system 1000 has access to a tag on a structure for data storage, such as, for example, an RFID tag. In some embodiments, the computing system 1000 manages the information of the tag, as well as an associated file system or database. Processor(s) 1001 may access these communication packets stored in memory 1003 for processing.

Examples of the network interface 1020 include, but are not limited to, a network interface card, a modem, and any combination thereof. Examples of a network 1030 or network segment 1030 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 1030, may employ a wired and/or a wireless mode of communication. In general, any network topology may be used.

Information and data can be displayed through a display 1032. Examples of a display 1032 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 1032 can interface to the processor(s) 1001, memory 1003, and fixed storage 1008, as well as other devices, such as input device(s) 1033, via the bus 1040. The display 1032 is linked to the bus 1040 via a video interface 1022, and transport of data between the display 1032 and the bus 1040 can be controlled via the graphics control 1021. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 1032, computer system 1000 may include one or more other peripheral output devices 1034 including, but not limited to, an audio speaker, a printer, a storage device, and any combinations thereof. In some instances, a peripheral output device 1034 may correspond to a tag on a structure, such as, for example, an RFID tag. Such peripheral output devices may be connected to the bus 1040 via an output interface 1024. Examples of an output interface 1024 include, but are not limited to, a serial port, a parallel connection, a USB port, a FIREWIRE port, a THUNDERBOLT port, PCi, fiber optic channel. SAS. SATA, and any combinations thereof.

In addition or as an alternative, computer system 1000 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers. Internet appliances, mobile smartphones, tablet computers, personal digital assistants, video game consoles, and vehicles.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®. Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

The systems provided herein may be controlled or monitored using a web application. In some embodiments, a computer program includes a web application. In some instances, a computer program for monitoring a data storage system or platform described herein is provided as a web application. The front-end of the web application may display one or more layers or elements that allows a user (e.g., technician or engineer) to interact, such as, for example, buttons, images, navigation menus, and text. The web application may further display an alert if a fault or malfunction is detected in the data storage system. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, XML, and document oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous JavaScript and XML (AJAX), Flash® ActionScript, JavaScript. or Silverlight. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein. Similar to the web application, the mobile application may be provided for monitoring a data storage system or platform described herein. The front-end of the mobile application may display one or more layers or elements that allows a user (e.g., technician or engineer) to interact, such as, for example, buttons, images, navigation menus, and text. The mobile application may further display an alert as a notification if a fault or malfunction is detected in the data storage system.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock. Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Stork, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, a distributed computing resource, a cloud computing resource, or combinations thereof. In further various embodiments, a software module comprises a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for one or more of storage, cataloging, and retrieval of information, such as information related to a system or platform (e.g., for data storage or biomolecule extraction) provided herein. In various embodiments, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, My SQL. Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices.

### Certain Definitions

Unless otherwise defined, all technical terms used herein have the same meaning as commonly understood by one of ordinary skill in the art to which the present subject matter belongs.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Reference throughout this specification to “some instances,” “further instances,” or “a particular instance,” means that a particular feature, structure, or characteristic described in connection with the instance is included in at least one instance. Thus, the appearances of the phrase “in some instances,” or “in further instances,” or “in a particular instance” in various places throughout this specification are not necessarily all referring to the same instance. Furthermore, the particular features, structures, or characteristics may be combined in any suitable manner in one or more instances.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

Polynucleotide sequences described herein may be, unless stated otherwise, comprise DNA or RNA or an analog or derivative thereof. As used herein, the terms nucleic acids, polynucleotides, oligonucleotides, oligos, oligonucleic acids are used synonymously throughout to represent a polymer of nucleoside monomers. In some instances, nucleic acids are connected via phosphate or sulfur-containing linkages. Nucleic acids in some instances comprise DNA, RNA, non-canonical nucleic acids, unnatural nucleic acids, or other nucleoside. In some instances, nucleotides comprise non-canonical bases, sugars, or other moiety. In some instances, nucleotides comprise terminators which are configured to prevent extension reactions. In some instances, such terminators are removed before addition of subsequent nucleotides to the growing chain.

The present disclosure is further described by the following non-limiting items.

Item 1. A system for data storage comprising:
(a) a computing system comprising at least one processor and instructions executable by the at least one processor to perform operations comprising:
   (i) receiving digital information;
   (ii) encoding digital information in one or more nucleic acid sequences; and
   (iii) synthesizing a library of polynucleotides corresponding to the nucleic acid sequences; and
(b) one or more modular rack-mount synthesis units.

Item 2. The system of item 1, wherein the rack mount synthesis unit is configured for user access.

Item 3. The system of item 1 or 2, wherein the rack mount synthesis unit is configured to slide laterally from a rack enclosure.

Item 4. The system of any one of items 1-3, wherein the rack mount synthesis unit comprises rollers, tracks, or chains attached to the synthesis unit.

Item 5. The system of any one of items 1-4, wherein the rack mount synthesis unit is configured to fit into an OCP rack.

Item 6. The system of item 5, wherein the OCP rack comprises a standard 539 mm (21.22″) wide equipment space with a 48 mm (1.89″) OpenU tall space.

Item 7. The system of any one of items 1-6, wherein the one or more synthesis unit comprises one or more of:
(a) a computer controller;
(b) one or more reservoirs;
(c) at least one flow cell block;
(d) an extraction stage;
(e) a post-processing unit;
(f) a storage unit;
(g) a robotic interface; and
(h) a rack interface.

Item 8. The system of item 7, wherein the flow cell block comprises at least 12 flow cells.

Item 9. The system of item 7 or 8, wherein the flow cell block comprises a spacing of no more than 100 mm.

Item 10. The system of any one of items 7-9, wherein the system comprises at least 8 flow cell blocks.

Item 11. The system of any one of items 7-10, wherein the flow cell block comprises:
one or more flow cells comprising:
   (a) one or more solid supports configured for polynucleotide synthesis; and
   (b) one or more ports for exchange of gases, synthesis reagents and/or extracted polynucleotides.

Item 12. The system of item 11, wherein at least one of the ports is controlled by a valve.

Item 13. The system of item 11 or 12, wherein each flow cell is configured for synthesizing a library of polynucleotides.

Item 14. The system of item 13, wherein synthesizing comprises one or more step of:
filling the flow cell with one or more reagents;
washing the flow cell with a solvent; and
drying the flow cell.

Item 15. The system of item 13 or 14, wherein synthesizing comprises coupling one or more nucleic acid monomers to one or more polynucleotides to generate the library of polynucleotides.

Item 16. The system of item 15, wherein the method further comprises one or more of:
deprotecting one or more polynucleotides attached to a surface;
oxidizing one or more polynucleotides attached to a surface;
capping one or more polynucleotides attached to a surface; and
extracting one or more polynucleotides from the surface.

Item 17. The system of item 16, wherein extracting comprises one or more of:
cleaning the flow cell;
filling the flow cell with one or more reagents; and
extracting the polynucleotide library from the flow cell.

Item 18. The system of any one of items 11-17, wherein the one or more flow cells have a pitch distance of no more than 9 mm.

Item 19. The system of any one of items 11-18, wherein the one or more flow cells are fluidically connected via a reagent distribution manifold.

Item 20. The system of any one of items 11-19, wherein the one or more flow cells are fluidically connected via at least one reagent distribution manifold.

Item 21. The system of item 20, wherein the at least one reagent distribution manifold is connected to one or more of a gas supply, flow waste, supply prime waste, and supply inlet.

Item 22. The system of any one of items 11-21, wherein the one or more flow cells comprise an input and an output port.

Item 23. The system of item 22, wherein the output port is configured to direct liquids to waste or an extraction stage.

Item 24. The system of any one of items 11-23, wherein the one or more flow cells comprise a volume of 50-200 microliters.

Item 25. The system of any one of items 11-24, wherein the one or more solid supports comprise a plurality of addressable loci.

Item 26. The system of item 25, wherein each addressable loci is configured to synthesize a polynucleotide having a different sequence.

Item 27. The system of item 7, wherein the extraction stage comprises:
an extraction stage e-chain (energy supply chain); and
one or more ports for exchange of synthesis reagents and/or extracted polynucleotides.

Item 28. The system of item 27, wherein at least one of the ports is controlled by a valve.

Item 29. The system of item 27 or 28, wherein the extraction stage is configured to performing an extraction and/or one or more post processing operations, wherein extraction operations occur in the liquid phase.

Item 30. The system of item 7, wherein the post-processing unit comprises:
an e-chain (energy supply chain); and
one or more ports for exchange of synthesis reagents and/or extracted polynucleotides.

Item 31. The system of item 30, wherein at least one of the ports is controlled by a valve.

Item 32. The system of item 30 or 31, wherein the extraction stage is configured to performing one or more post processing operations, wherein post processing operations occur in the liquid phase.

Item 33. The system of item 32, wherein one or more post processing operations comprise drying, concentration, amplification, ligation, digestion, deprotection, or one or more quality control processes.

Item 34. The system of item 33, wherein one or more quality control processes comprises analysis of
(a) concentration;
(b) length;
(c) yield;
(d) melting temperature;
(e) conductivity; or
(f) hybridization.

Item 35. The system of item 33 or 34, wherein one or more quality control processes comprises:
(a) qPCR;
(b) amplification;
(c) sequencing;
(d) measuring UV adsorption; or
(e) gel electrophoresis.

Item 36. The system of item 6, wherein the storage unit comprises:
(a) one or more storage plates;
(b) a robotics interface configured to move storage plates; and
(c) an access port.

Item 37. The system of item 36, wherein the storage plate comprises at least 90 wells.

Item 38. The system of item 36 or 37, wherein the storage plate comprises polynucleotides associated with a specific flow cell.

Item 39. The system of item 6, wherein the rack interface comprises:
(a) a power supply;
(b) a rack reagent bulkhead;
(c) a synthesis unit reagent bulkhead;
(d) one or more reservoirs;
(e) a reagent e-chain (energy supply chain); and
(f) a pilot valve bank.

Item 40. The system of item 39, wherein the power supply comprises a 24V power supply.

Item 41. The system of item 39 or 40, wherein the reagent e-chain comprises 1/16″ ID tubing.

Item 42. The system of any one of items 39-41, wherein the rack interface comprises one or more panels.

Item 43. The system of item 42, wherein the rack interface comprises one or more of a front panel, rear panel, top panel, bottom panel, and side panel.

Item 44. The system of item 43, wherein the panel comprises interfaces for one or more of liquid reagents, gases, and electrical communication.

Item 45. The system of item 44, wherein the interfaces comprise ports for one or more of extraction, coupling reagents, bulk reagents, and waste.

Item 46. The system of any one of items 43-45, wherein the panel comprises interfaces for at least two gas pressures.

Item 47. The system of any one of items 43-46, wherein the panel comprises interfaces for at least three gas pressures.

Item 48. The system of any one of items 43-47, wherein interface for electrical communication comprises at least one of a power supply and a communications interface.

Item 49. The system of item 48, wherein the communications interface comprises a wireless communications interface.

Item 50. The system of item 48, wherein the communications interface comprises USB.

Item 51. The system of any one of items 43-50, wherein the panel comprises an access panel.

Item 52. The system of item 51, wherein the access panel comprises a transparent material.

Item 53. The system of item 51, wherein the access panel comprises a single pane of glass.

Item 54. The system of any one of items 51-53, wherein the access panel is configured to enable one or more of system monitoring, fault evaluation, identification, and field service capability.

Item 55. The system of item 12, wherein the valves are controlled by a pilot valve bank.

Item 56. The system of item 55, wherein the pilot valve bank comprises at least 24 channels.

Item 57. The system of item 45, wherein bulk reagents comprise one or more of capping reagent, deblocking reagent, oxidation reagent, and solvents.

Item 58. The system of item 57, wherein solvents comprise acetonitrile, acetone, or a mixture thereof.

Item 59. The system of item 45, wherein coupling reagents comprises one more of activator, linker, and nucleotide monomer.

Item 60. The system of item 45, wherein extraction reagents comprise one or more of tert-butylamine (TBA) and one or more solvents.

Item 61. The system of item 60, wherein one or more solvents comprises water, acetonitrile, or a mixture thereof.

Item 62. The system of any one of items 1-61, wherein the system further comprises a sequencing unit.

Item 63. The system of item 62, wherein the sequencing unit is configured to sequence polynucleotide libraries.

Item 64. The system of any one of items 1-63, wherein the polynucleotide libraries are obtained from the storage unit.

Item 65. The system of any one of items 1-64, wherein the system further comprises one or more system monitoring modules.

Item 66. The system of item 65, wherein the one more system monitoring modules comprise one or more sensors.

Item 67. The system of item 66, wherein the one or more sensors are configured to detect temperature, pressure, humidity, changes in voltage or current, capacitance, conductivity, storage plate position, liquid volume, flow rate, or presence of liquids.

Item 68. The system of item 66 or 67, wherein the one or more sensors are configured to report a status to the computer system.

Item 69. The system of item 68, wherein the status comprises a value and/or fault condition.

Item 70. The system of any one of items 66-69, wherein the one or more sensors are configured to disconnect main power to the data storage system.

Item 71. The system of any one of items 66-70, wherein the one or more sensors are configured to pause synthesis at one or more flow cell blocks.

Item 72. The system of any one of items 66-70, wherein the one or more sensors are configured to pause synthesis at one or more flow cells.

Item 73. The system of item any one of items 66-72, wherein at least one sensor configured to detect the presence of liquid is located near the base or bottom of the system.

Item 74. The system of item any one of items 66-72, wherein at least one sensor configured to detect the presence of liquid is located near the base or bottom of a rack.

Item 75. The system of item 6, wherein the controller comprises one or more printed circuit board assemblies (PCBAs).

Item 76. The system of item 75, wherein the one or more PCBAs comprises a control, mother1, mother2, or field programmable array gate (FPGA).

Item 77. The system of item 76, wherein at least 12 flow cells are controlled per FPGA.

Item 78. The system of any one of items 1-77, wherein the system is configured to store at least 1 petabyte (PB) of data.

Item 79. The system of any one of items 1-77, wherein the rack mount synthesis unit is configured to store at least 10 petabyte (PB) of data.

Item 80. The system of any one of items 1-77, wherein the rack mount synthesis unit is configured to store at least 1 exabyte (EB) of data.

Item 81. The system of any one of items 1-80, wherein the system is configured for fault tolerance.

Item 82. The system of item 81, wherein the computer system is configured to divert encoded data from a paused flow cell or flow cell block to an active flow cell or flow cell block, respectively.

Item 83. The system of item 81, wherein the computer system is configured to divert encoded data from a paused flow cell or flow cell block to a different data storage system.

Item 84. The system of item 81, wherein the computer system further comprises a cache.

Item 85. The system of item 81, wherein digital information is retained in the cache until polynucleotides encoding the digital information are stored.

Item 86. A storage network comprising:
one or more of:
   (a) one or more systems of any one of items 1-85;
   (b) one or more magnetic media storage devices; and
   (c) one or more solid-state media storage devices; and
a network system configured for data transfer between (a)-(c).

Item 87. An assembly for storing information comprising:
(a) a plurality of compartments comprising a first material;
(b) a base plate comprising a second material; and
(c) at least one cover plate comprising a third material, wherein a coefficient of thermal expansion (CTE) of the first material, the second material, or both is less than the third material.

Item 88. The assembly of item 87, wherein the plurality of compartments are positioned in an array on the base plate.

Item 89. The assembly of item 87 or 88, wherein the plurality of compartments are separable from the base plate.

Item 90. The assembly of item 87 or 88, wherein the plurality of compartments are fixed on the base plate.

Item 91. The assembly any one of the preceding items, wherein the cover plate, the base plate, or both comprise a plurality of recessed features.

Item 92. The assembly of item 91, wherein each wall of the recessed feature is at least partially angled at no more than 10.

Item 93. The assembly of item 91 or 92, wherein each wall of the recessed feature of the cover plate and an outer wall of a compartment is separated no more than about 5 μm.

Item 94. The assembly of any one of items 91-93, wherein each compartment is located wholly or partially within a recessed feature of the base plate, the cover plate, or both.

Item 95. The assembly of any one of items 91-94, wherein each of the recessed feature of the cover plate or the base plate has a diameter of about 1 mm to 5 mm.

Item 96. The assembly of any one of items 91-95, wherein the plurality of recessed features is separated by a pitch distance of about 3 mm to about 5 mm.

Item 97. The assembly of any one of items 91-96, wherein each wall between each of the recessed features of the cover plate or the base plate has a thickness of about 0.5 mm to about 3 mm.

Item 98. The assembly of any one of items 91-97, wherein each wall of the recessed feature of the cover plate or the base plate has a length of about 1 mm to about 10 mm.

Item 99. The assembly of any one of the preceding items, wherein a wall of each of the compartments comprises a thickness of about 0.1 mm to about 2 mm.

Item 100. The assembly of any one of items 95-98, wherein the diameter, the pitch distance, the length, or the thickness comprises a tolerance of about 10 μm or less.

Item 101. The assembly of any one of items 95-100, wherein the diameter, the pitch distance, the length, or the thickness comprises a tolerance of about 5 μm or less.

Item 102. The assembly of any one of the preceding items, wherein the assembly comprises at least two cover plates.

Item 103. The assembly of item 102, wherein each of the at least two cover plates seals about six compartments of the plurality of compartments.

Item 104. The assembly of any one of the preceding items, wherein the second material, the third material, or both comprise a metal.

Item 105. The assembly of any one of the preceding items, wherein the second material comprises a high specific heat capacity material.

Item 106. The assembly of item 105, wherein the high specific heat capacity material comprises a specific heat capacity of about 0.5 J/k-° C. to 2.5 J/k-° C.

Item 107. The assembly of any one of the preceding items, wherein the first material, the second material, or both is inert.

Item 108. The assembly of any one of the preceding items, wherein the first material comprise borosilicate.

Item 109. The assembly of any one of the preceding items, wherein the third material comprises low emissivity.

Item 110. The assembly of any one of the preceding items, wherein the third material comprises stainless steel.

Item 111. The assembly of any one of the preceding items, wherein the third material comprises a CTE of about 15 μm/m-° C. to 20 μm/m-° C.

Item 112. The assembly of any one of the preceding items, wherein the third material comprises a thermal conductivity of about 15 W/m-K to about 20 W/m-K.

Item 113. The assembly of any one of the preceding items, wherein the third material comprises a specific heat capacity of about 0.5 J/g-° C.

Item 114. A method for storing information comprising:
(a) providing an assembly comprising:
   (i) a plurality of compartments, wherein the plurality of compartments comprise a plurality of polynucleotides;
   (ii) a base plate; and
   (iii) a cover plate;
(b) generating a temperature gradient between the base plate and the cover plate, wherein the temperature gradient causes the base plate, the cover plate, or both to expand or contract to seal the plurality of compartments.

Item 115. The method of item 114, wherein the plurality of compartments are arranged in an array on the base plate.

Item 116. The method of item 114 or 115, wherein the method further comprises transferring the plurality of polynucleotides to the plurality of compartments prior to (a).

Item 117. The method of item 116, wherein transferring comprises transferring the plurality of polynucleotides in a solution.

Item 118. The method of item 116 or 117, wherein transferring comprises depositing one or more droplets comprising the plurality of polynucleotides using a nozzle of a deposition system.

Item 119. The method of any one of the preceding items, wherein each compartment of the plurality of compartments comprises a volume of a solution comprising the plurality of polynucleotides.

Item 120. The method of item 119, wherein the volume is about 10 μL to about 200 μL.

Item 121. The method of any one of the preceding items, wherein the base plate or the plurality of compartments are not in contact with the cover plate in (a).

Item 122. The method of any one of the preceding items, wherein the method further comprises positioning the cover plate above the base plate or the plurality of compartments.

Item 123. The method of item 122, wherein the cover plate is positioned above the base plate or the plurality of compartments using a piezo stage, vision system, linear motor, or rotary motor.

Item 124. The method of any one of the preceding items, wherein generating the temperature gradient comprises cooling or heating the base plate, the cover plate, or both.

Item 125. The method of item 124, wherein generating the temperature gradient comprises sequentially changing a temperature of the base plate and the cover plate.

Item 126. The method of item 124, wherein generating the temperature gradient comprises simultaneously changing a temperature of the base plate and the cover plate.

Item 127. The method of any one of the preceding items, wherein generating the temperature gradient comprises one or more operations comprising:
(i) cooling the base plate;
(ii) heating the cover plate;
(iii) placing the cover plate on the base plate comprising the plurality of compartments; and
(iv) returning the cover plate, the base plate, or both to ambient temperature.

Item 128. The method of item 127, wherein (i) and (ii) are performed simultaneously.

Item 129. The method of item 127, wherein (i) and (ii) are performed sequentially.

Item 130. The method of any one of the preceding items, wherein the base plate is cooled to about −50° C. to 50° C.

Item 131. The method of any one of the preceding items, wherein the cover plate is heated to about 20° C. to about 100° C.

Item 132. The method of any one of the preceding items, wherein the base plate contracts by about 1% to 25% in (i).

Item 133. The method of any one of the preceding items, wherein the cover plate expands by about 1% to 25% in (ii).

Item 134. The method of any one of the preceding items, wherein the temperature gradient is monitored radiantly, inductively, or resistively.

Item 135. The method of any one of the preceding items, wherein the temperature gradient is monitored by sensor for measuring one or more of temperature, emission, or resistance.

Item 136. The method of any one of the preceding items, wherein the method further comprises drying the plurality of polynucleotides.

Item 137. The method of any one of the preceding items, wherein the method further comprises retrieving the plurality of polynucleotides or a portion thereof.

Item 138. The method of item 137, wherein retrieving comprises removing the cover plate in contact with the plurality of containers, the base plate, or both.

Item 139. The method of item 137 or 138, wherein retrieving comprises heating the cover plate or a portion thereof.

Item 140. The method of any one of items 137-139, wherein retrieving comprises cooling the base plate or a portion thereof.

Item 141. The method of item 137, wherein retrieving comprises accessing the plurality of polynucleotides without removing the cover plate in contact with the plurality of containers, the base plate, or both.

Item 142. The method of item 141, wherein retrieving comprises piercing a portion of the cover plate with a needle.

Item 143. The method of any one of items 137-142, wherein retrieving comprises dissolving the plurality of polynucleotides in a solution.

Item 144. A system for storing information comprising:
(a) an assembly for storing information, the assembly comprising:
   (i) a plurality of compartments;
   (ii) a base plate; and
   (iii) at least one cover plate;
(b) a material deposition system comprising a dispenser, wherein the dispenser deposits a plurality of polynucleotides to a compartment of the plurality of compartments;
(c) one or more temperature control systems for heating or cooling the base plate, the cover plate, or both; and
(d) a computing system comprising at least one processor and instructions executable by the at least one processor to perform one or more operations, the one or more operations comprising:
   (i) orchestrating movement of one or more components of the system; and
   (ii) monitoring a property of the one or more components of the system.

Item 145. The system of item 144, wherein the one or more components comprises the cover plate, the base plate, the material deposition system, or the one or more temperature control systems.

Item 146. The system of item 144 or 145, wherein the plurality of compartments comprises a first material, the base plate comprises a second material, or the cover plate comprises a third material.

Item 147. The system of item 146, wherein a coefficient of thermal expansion (CTE) of the third material is less than the second material.

Item 148. The system of any one of the preceding items, wherein the dispenser deposits a plurality of droplets comprising the plurality of polynucleotides to the compartment of the plurality of compartments.

Item 149. The system of any one of the preceding items, wherein orchestrating the movement of one or more components of the system comprises positioning the cover plate over the base plate, placing the cover plate on the base plate, or both.

Item 150. The system of any one of the preceding items, wherein orchestrating the movement of one or more components of the system comprises aligning the dispenser of the material deposition system to a compartment of the plurality of compartments.

Item 151. The system of any one of the preceding items, wherein the property comprises temperature, humidity, pressure, salinity, light sensor, UV, O₂, or any combination thereof.

Item 152. The system of any one of the preceding items, wherein monitoring the property of one or more components comprises monitoring the temperature of the base plate, the cover plate, or both.

### EXAMPLES

The following illustrative examples are representative of embodiments of the software applications, systems, and methods described herein and are not meant to be limiting in any way.

### Example 1: Electrochemical Synthesis Device

An electrochemical polynucleotide synthesis device was fabricated according to the general methods and examples disclosed in U.S. Patent Publication No. 2022/0064206, which is incorporated by reference in its entirety.

### Example 2: Automated Data Storage System

Using the general methods of Example 1, a modular data storage system is assembled with functions as described in FIGS. 1A-1B and FIG. 2B. The system comprises separate dedicated modules for DNA synthesis (writing), post processing, long term storage and cataloging, and optionally DNA sequencing (reading) (FIG. 1C). The synthesis module consists of many tens to hundreds of individual, independently addressable flow cells (FIG. 2A), each of which contains one DNA synthesis chip (e.g., Clio). There are several potential advantages to the combination of one chip—containing many millions to billions of synthesis cavities—per flow cell, and a large number of autonomous flow cells per synthesis module. Firstly, it significantly increases the rate at which data can be written (synthesized). Secondly, it enables the use of liquid extraction of the DNA synthesized by each individual flow cell into a separate and uniquely identifiable compartment (well or capsule) located in a separate (removeable) plate. Thirdly, it enables the use of an independent robotic system to move plates between and within the individual modules located within the rack—for synthesis, post-processing, sealing and storage (see figures). Fourthly, it enables end-to-end data protection—each flow cell can be completely instrumented using multiple sensors which monitor in real time such conditions as, for example, internal temperature and pressure, rate of flow of reagents through the cell, electronic functionality for each synthesis well, interface health and proper communication protocols and the cell's external environmental conditions. By providing real time process conditions for each individual cell, the system can determine the successful completion of any cell's synthesis cycle, flag (for repeat or duplicate runs with alternate flow cells) and mark for replacement, and service any flow cell whose performance is detected to be sub-optimal. In some instances, provided herein are systems wherein a robotic or automated platform is configured to replace any data storage system described herein (e.g., flow cell, flow cell block, reservoir, single rack unit, or other component).

An isometric side view of the synthesis unit inside a rack is shown in FIG. 3A, with the unit in the pulled-out or user-accessible position. A detailed view of the synthesis unit is shown in FIG. 3B showing electrical components (power supply, four PCBAs), eight flow cell blocks (with 12 flow cells per block), reagent bulkheads, a 24-channel pilot valve bank. DNA storage unit, and e-chains for reagent and extraction delivery. A transparent front view of a rack-mount synthesis unit is shown in FIG. 3C. The rear panel 307 (e.g., reagent bulkhead) of the synthesis unit is shown in FIG. 3D, and includes ports for extraction, amidites for synthesis, bulk reagents, three different nitrogen pressures (N2-1, N2-2, N2-3), a port for waste, a power connector, and a communications port for data transfer (optionally USB 3.0). A side cutout view of the synthesis device showing e-chain locations, dispense/waste locations, and a 96-well plate for DNA storage is shown in FIG. 4A. A robotics stage moves a storage unit comprising a 96-well plate to different flow cell block positions in the synthesis unit. FIG. 4B depicts a fluidics diagram of reagents through the synthesis device from the reagent e-chain to a reagent distribution manifold fluidically connected to the flow cell blocks, as well as a manifold bypass to waste. FIG. 4C depicts a fluidics diagram from the reagent distribution manifold to all 8 individual flow cell blocks. Each flow cell block comprises 12 flow cells. The length of all eight flow cell blocks is 700 mm. Each flowcell block also comprises an inlet for nitrogen and an outlet for waste. FIG. 4D depicts a detail top view of a single flow cell block including reagent and nitrogen valve positions. FIG. 4E depicts another view of a single flow cell block with inputs for reagents and nitrogen shown. FIG. 6 depicts a fluidics diagram for a flow cell block, including routing and valve positions for a single flow cell in the inset.

FIG. 5A depicts the operation of a single flow cell in a flow cell block during DNA synthesis. After each step, reagents may be flowed to waste. First, the flow cell is filled with a phosphoramidite and activator to couple a nucleoside to a polynucleotide chain on a surface within the flow cell. Electrochemical masking control (e.g., by controlling which loci comprise free OH groups for coupling) of each addressable loci on the surface allows only specific loci to couple the phosphoramidite. After coupling, bulk reagents and one or more wash solvents are used to prepare the surface for another coupling step using a different phosphoramidite. The surface is also dried. A new mask is also applied to the surface through electrochemical generation of acid at specific loci which have not yet been coupled. The acid deprotects polynucleotides at these new positions by exposing free OH groups. After all four phosphoramidites have been added to loci on the support (e.g., corresponding to A, T, G. C) a “layer” has been completed. Additional steps include oxidation and capping. This process is completed until the polynucleotides of the desired sequence and length are synthesized.

FIG. 5B depicts extraction of polynucleotides (e.g., a polynucleotide library) from the flow cell after synthesis is completed. DNA extraction occurs entirely in the liquid phase to facilitate transfer and storage.

Utilizing a modular, fluidically coupled system level concept in some instances provides flexibility and scalability, while simplifying the design (and engineering development) to individual functional blocks. For example, the design in some instances addresses the module form factor and centralized/standardized connectivity of the individual DNA synthesis units (flow cells) which can enable advances in synthesis technologies to be easily incorporated in the overall storage system, resulting in generational increases in storage capacities and throughput within the standard (data center) rack scale form factor. In addition, by aggregating the individual synthesis units (flow cells) across a common reagent/chemical delivery and recovery platform, which can be located remotely from the DNA data storage rack, the delivery of the required reagents to the system can be simplified for process monitoring, fault tolerance and (isolated and accessible) servicing.

The incorporation of a fully automated robotic plate handling system enables the separation of synthesis modules from post-processing, sealing and storage modules also simplifying design field servicing and replacement/upgrading. The robotic systems is fully integrated within the storage system control software/firmware (and external host applications) and these are capable of cataloguing all storage capsules/trays loaded, unloaded and stored within the rack. Designing these robotic systems around a standard (and potentially proprietary) storage (and intermediate) tray design enables the system design costs to be amortized (and monetized) over many generations. Finally, developing a standardized, modular system significantly improves field serviceability, system maintenance and the incorporation of generational performance improvements.

Once synthesized, the DNA is extracted (purged) from the individual flow cells into a single dedicated structure for storage (e.g., storage chamber) which in some instances is sealed after the completion of any necessary ancillary processing steps. Each chamber and tray is capable of being catalogued and tracked by the DNA storage system and this information (metadata) is available to the host applications. A structure for storing a plurality of polynucleotides encoding for digital information may comprise coatings on one or more surfaces. In some instances, coatings are present on the inside surface of a structure (not in contact with the outside environment when the structure is closed/sealed). In some instances, coatings are present on the outside surface of a structure (exposed to environment). A structure in some instances comprises one or more coatings, such as 1, 2, 3, 4, 5, or more than 5 coatings. Coatings in some instances comprise similar materials, or alternatively at least some of the coatings comprise different materials. Various coatings in some instances provide one or more properties to the surface of the structure such as increased resistance to corrosion, desiccation, hydrophobicity, oxygen absorption, or other property conducive to polynucleotide storage. Exemplary coatings include but are not limited to coatings that comprise plastics, synthetic polymers, glass, silica, metals, biological polymers, proteins, or other material. In some instances, the solid support, the surface, or both comprise a material described herein. In some instances, the material comprises a metal or organic polymer. In some instances, the material comprises steel (e.g., stainless steel) or other metal alloy. In some instances, the material comprises polyethylene, polypropylene, or other polymer. In some instances, the structure comprises a flexible material, such as those provided herein. Exemplary flexible materials include, without limitation, modified nylon, unmodified nylon, nitrocellulose, and polypropylene. In some instances, the materials comprise a rigid material, such as those provided herein. Exemplary rigid materials include, without limitation, glass, fuse silica, silicon, silicon dioxide, silicon nitride, plastics (e.g., polytetrafluoroethylene, polypropylene, polystyrene, polycarbonate, and blends thereof, and metals (e.g., steel, gold, platinum). In some instances, materials disclosed herein may be fabricated from a material comprising silicon, polystyrene, agarose, dextran, cellulosic polymers, polyacrylamides, polydimethylsiloxane (PDMS), glass, or any combination thereof. In some examples, materials disclosed herein are manufactured with a combination of materials listed herein or any other suitable material known in the art.

The system robotics are capable of moving storage trays to and from the flow cells and then moving the trays from the synthesis module(s) to the storage module(s). In some instances, an extraction stage comprises a robotics system. Empty storage trays are loaded and unloaded into the DNA data storage rack (via a dedicated and controlled access port) and shall be both shock and vibration resistant (c.f, a conventional tape cartridge “drop test”) with the individual capsules/wells locked into position within the tray. Depending on the degree of synthesis/sequencing integration that is required, some manual operations may be employed. For example, loading and unloading DNA data storage trays into the system and replacement of the DNA synthesis units as necessary. An exemplary arrangement for a robotics system is shown in FIG. 4A. A 96-well plate 317 in some instances is moved along an axis from dispense location #1 (319 top diagram) to waste/wash location #8 (320, bottom diagram). The plate may also be moved to any location corresponding to one or more flow cell block positions. In some instances a robotics system moves a storage chamber along a stage travel. In some instances, a stage travel comprise a distance of 100-5000 mm, 100-3000 mm, 100-2500, 100-2000, 100-1500, 100-1000, 250-5000, 250-2500, 250-1500, 250-1000, 500-2000, 500-3000, 500-5000, 700-1500, 700-1000, 700-900, 1000-5000, 1000-3000, or 1000-2000 mm.

In each case the tray and capsules are catalogued and are fully traceable with the contents of the capsules encrypted and encoded by a customer. For an on-premises solution the rack level system in some instances integrates both synthesis and sequencing modules in a single rack.

### Example 3: Writing Metadata to an RFID Tag

Digital information of about 18 GB is encoded as sequences of a plurality of polynucleotides and the plurality of polynucleotides are synthesized using the methods described herein. Here, a synthesis chip with about one billion cells produces 240-mer oligonucleotides. An ECC is applied to the sequences of the plurality of polynucleotides, which imposes a 40% overhead. The capsule then holds about 140×10⁹ bits of data, which is about 18 GB. The plurality of polynucleotides are then transferred to a structure for storage. The structure comprises a capsule with an interval storage volume of about 200 μL.

The capsule comprises an RFID tag embedded in the structure. The RFID tag is a passive tag with an ultra-high frequency range of about 860 to about 960 MHz. The RFID tag includes an integrated circuit or chip for storing and processing information, modulating and demodulating a RF signal, as well as a tag antenna. A reader (also referred to as a transceiver) including a reader antenna is used to transmit a modulated RF signal to the RFID tag. The tag antenna receives the RF signal and forms an electric and magnetic field from which the RFID tag draws power for the integrated circuit. The integrated circuit then causes the RFID tag to modulate a backscatter RF signal back to the tag reader, and the RF signal containing information is encoded in the memory of the RFID tag.

The information contained in the RFID signal is metadata relating to the plurality of polynucleotides stored on the RFID tag. The metadata comprises data type and data size of the digital information, date of synthesis, owner information, manufacturer information, storage mechanism, and storage location. The metadata is about 1 to about 2 kbits.

The capsule with an RFID tag is then stored on an identifiable layout, such as a rack. The storage location encoded on the RFID tag matches a location on the identifiable layout that the capsule is placed. The capsule is placed on a given shelf at a given row and lane on the rack, which is the storage location encoded in the metadata of the RFID tag. The rack can house about 1000 capsules, where each capsule has a unique RFID tag encoding metadata specific to that capsule.

### Example 4: Reading Metadata on an RFID Tag

A capsule storing a plurality of polynucleotide encoding digital information is stored on an identifiable layout, such as a rack. The rack hoses about 1000 capsules. Each capsule includes an RFID tag as previously described in Example 3. The digital information stored on the capsule can be retrieved by first accessing the RFID tag.

A reader (also referred to as a transceiver) including a reader antenna is used to transmit a modulated RF signal to the RFID tag. The modulated RF signal can contain an identifying number. The tag antenna receives the RF signal and forms an electric and magnetic field from which the RFID tag draws power for the integrated circuit. The integrated circuit or chip can verify the identifying number, and if correct, can transmit encoded information back to the tag reader by modulating a backscatter RF signal back to the tag reader.

Each RFID tag includes about 1 to 2 kbits of data. The reader can scan up to about 848 kbits per second, allowing for reading multiple RFIG tags from a capsule in parallel (e.g., up to about 800 capsules at once). The reader is about 18 inches from a shelf on the rack. The reader then identifies the capsule of interest based on the metadata stored on the RFID tag. For example, the reader identifies a given file number stored as metadata on the RFID tag.

The capsule is then retrieved from the rack. The plurality of polynucleotides stored in the capsule are amplified and sequenced using the methods described herein. An ECC is then applied to the sequences of the plurality of polynucleotides to retrieve the encoded digital information.

### Example 5: Determining Data Integrity Based on an RFID Tag

A plurality of capsules each storing a plurality of polynucleotides encoding digital information is stored on an identifiable layout as described in Example 4. Each capsule includes an RFID tag. Metadata related to the plurality of polynucleotides in each of the capsules is written to each of the RFID tags on the capsules, which is described in Example 3.

Data integrity of the information stored in the capsules is determined after a duration of time of about 5 years. The data integrity is determined by scanning each of the RFID tags on each of the capsule using a reader. The reader scans multiple RFID tags in parallel as described in Example 4, above.

An RFID tag is scanned and deemed valid or invalid. A valid RFID tag is an RFID tag on a capsule that is scanned with no errors and metadata stored in the RFID tag can be retrieved. An invalid RFID tag is an RFID tag on a capsule that has been damaged, such as structural damage to the RFID tag or the capsule. An RFID tag is also invalid if the capsule has been accessed or tampered with by an unauthorized user.

### Example 6: Removal of a Rack of Capsules from a Storage System

The plurality of capsules on a rack (or tray) from Example 3 are retrieved. Each of the capsules stores a plurality of polynucleotide encoding digital information. The rack comprising the plurality of capsules may be retrieved from the storage system for any number of reasons provided herein. For example, rack comprising the plurality of capsules may be retrieved to transfer it to a different storage system for long-term storage, for sequencing, for copying the digital information encoded in the plurality of polynucleotides to maintain fixity, or converting it to a secondary storage system.

Upon retrieval of the rack from the storage system, the RFID tags on each of the capsules is scanned as described in Example 4. When the RFID tags are scanned, a file system or a database is updated to reflect the retrieval and/or transfer of the capsule from one location to another. In addition, the RFID tags are updated with information pertaining to the removal process. Information pertaining to the removal process can comprise a date and time, environmental information, permission used for the extraction and identifying information for the removal requestor, any other pertinent detail to the retrieval and/or transfer, or any combination thereof.

By updating the system information, the file system or database keeps track of the available storage capacity and any relevant information pertaining to the storage system. For example, the file system or database keeps track of the empty slots or racks in the storage system, as well as the positioning of both trays and capsules, such as whether they are filled are empty.

### Example 7: Loading a Rack of Capsules into the Storage System

A file system or database in the data storage system keeps track of the storage capacity, as described in Example 6. In order for the data storage system to keep track of the available capacity, such as the number of available racks or slots (e.g., empty racks or slots), the data storage system updates the database (e.g., control applications database) when a new rack or new capsules are loaded into the system.

The RFID tags on the capsules that are loaded into the system are scanned and the metadata updated with relevant information. The information can include a time and date the rack was loaded, condition and manufacturing data of the capsules and/or rack, any environmental information, or any combination thereof. Once the new rack or capsules are loaded, control application(s) determine the disposition of the rack within the storage system and direct the robotic system to the correct position in the storage system.

### Example 8: Long Term DNA Data Storage Encapsulation

A plurality of polynucleotides encoding digital information as generally described in Example 3 are prepared and stored in a system for long term DNA data storage encapsulation as generally illustrated in FIGS. 15-18.

384 small borosilicate glass vials (or compartments) are arranged in a 24×16 array on common centers. The wells are positioned in shallow circular indentations in the base and/or cooling plate of an assembly, as shown in FIG. 18. This number of vials, and their positioning, are aligned with a standard (SBS) 384 well plate, thereby facilitating integration with conventional systems but, depending on the application, these numbers and dimensions may be modified. To avoid undue stress on the vials, as the cover plate is cooled (and, in some instances, commensurately the base and/or cooling plate is warmed), the cover plate encloses a 6×4 array of vials; and hence to encapsulate the full 384 vial array, sixteen cover plates is required.

Depending on the data contained in each vial, this division of data set can define a given unit of storage, such that for example, if each vial contains 64 GB of user data, then the 24 vials encapsulated by a single (6×4) cover plate would contain 1.536 TB and the sixteen cover plates would contain 24.576 TB. However, this compartmentalization can be adjusted depending on use case and data set size to optimize recall (sequencing) and data management.

To facilitate cooling of the vials (or compartments), and rapid quenching of the cover plate once positioned over the vials, the cooling plate is manufactured from a high specific heat capacity material, e.g. aluminum 0.921 J/k-° C., Beryllium 1.825 921 J/k-° C. Magnesium 1.047 921 J/k-° C., or other metal or alloy, consistent with the material not being deleterious to DNA in solid or solution.

The borosilicate of the vial is selected to meet three requirements: i) it is inert and will not contaminate or damage the stored DNA even over very extended periods; ii) its CTE is closely matched to the material of the cover plate; and iii) it has a relatively high thermal conductivity to mitigate cracking when the cover plate is rapidly cooled.

The material of the cover plate is also selected to be inert and not contaminate the desiccated DNA contained within the glass vials. A material with a high CTE is selected to maximize the strength (radial and tangential) of the compression seal formed when the cover plate cools. In addition, a material with a low emissivity is selected (e.g., the measure of an object's ability to emit infrared energy which can have a value from 0 (shiny mirror) to 1.0 (blackbody)). To maintain a low cost and consistency with existing proven long term storage methods, the cover plate is manufactured from stainless steel (e.g., 304 or 305, though other grades could be utilized). Emissivity factors for common stainless steels are provided in Table 1, below. The temperature coefficient of expansion for this material is 17.3 μm/m-° C. (0-100° C.), the thermal conductivity is 16.2 W/m-K (100° C.) and the specific heat capacity is 0.500 J/g-° C. (0.000-100° C.).

TABLE 1

| MATERIAL | TEMP ° F. (° C.) | e-EMISSIVITY |
|---|---|---|
| Type 301, Polished | 75 (24) | .27 |
| Type 301, Polished | 450 (232) | .57 |
| Type 316, Polished | 75 (24) | .28 |
| Type 316, Polished | 450 (232) | .57 |

### General Method of Encapsulation Workflow:

The general workflow using the system described herein is provided below:

(1) The glass vials are located on the base and/or cooling plate of an assembly and positioned by a robotic automated plate handling system to a location where they can be filled with a predetermined quantity of DNA in solution.

(2) The vials are filled (e.g., aliquoted) either singularly or in combination with a predetermined quantity of DNA in solution in the range 10 μL to 200 μL. Referring to FIG. 18, with the dimensions and tolerances shown, the DNA in solution has volume less than 36 μL.

(3) The DNA in solution is then dried using methods generally utilized in biotechnology research, development, or manufacturing, e.g., freeze drying, centrifuging or a combination thereof.

(4) Once dried to a predetermined level, the plate and vials are positioned on the cooling station where the base plate (and vials) is cooled using various methods, such as cryogenic cooling, Peltier cooling, mechanical refrigeration, or an alternative method known in the art.

(5) The cover plate is then positioned over the top of the base plate and vials with a low tolerance, but vertically separated from the top edge of the vial openings. This is done to reduce insofar as possible radiant heating of the desiccated DNA once the cover plate is heated.

(6) The cover plate is positioned with at least micron accuracy, registered to the position of the vials such that, once heated, when it is pushed onto the vials, the tapped holes (or recessed features) in the cover plate are accurately registered with the top opening of the vials. This can be achieved with various systems such as for example, piezo stages, vision systems, linear or rotary motors, or an alternative system known to those skilled in the art.

(7) The cover plate is then rapidly heated to a predetermined temperature, such that the plate expands by a known amount, and the temperature is monitored throughout this process. Heating can be achieved radiantly, inductively, and/or resistively, and the temperature is monitored via thermocouple, emission, or resistance, or al alternative method known in the art.

(8) Once at temperature, the heat source is turned off, and the cover plate is then pushed onto the vials to a set level or position where it cools and shrinks consistent with the temperature of the vials and base plate. As the cover plate shrinks, it forms a compressive seal around the outer surface of the vial, the stress of which can be determined from Lame's equations, and the known material properties and temperatures used for the cover plate and vials.

(9) Once the cover plate (and vials) have been cooled, the base plate (as well as vials and cover plate) is allowed to warm to ambient (room temperature) conditions, and the cover plate is moved by the automated robotic system to, as necessary, have a base (under) cover fitted to the glass vials for protection and transportation.

### Example 9: Long Term DNA Data Storage System

Polynucleotides that are stored using the structures and methods generally illustrated in Example 4 are stored in a storage system, such as a data center.

In some examples, the sealed device as generally provided in Example 4 is integrated onto a rack unit (e.g., FIGS. 3A-3C), which is conveniently inserted or removed from a server rack. The rack can house a number of devices or compartments, with mechanical structures commonly used for mounting conventional computing and data storage resources in rack units. For example, a rack may comprise openings adapted to support disk drives, processing blades, and/or other computer equipment.

Polynucleotides (and the information stored in them) contained in compartments can be accessed from the rack unit. Access can include removal of polynucleotides from the compartments, removal of compartments from the base plate, or removal of the device itself for analysis of polynucleotides in compartment to identify the information stored in the polynucleotides. Information in some instances is accessed using a robotic system. Information in some instances is accessed from a plurality of racks, a single rack, a device in a rack, a portion of the device or compartment.

While preferred embodiments of the present subject matter have been shown and described herein, it will be obvious to those skilled in the art that such embodiments are provided by way of example only. Numerous variations, changes, and substitutions will now occur to those skilled in the art without departing from the present subject matter. It should be understood that various alternatives to the embodiments of the present subject matter described herein may be employed in practicing the present subject matter.

1. A system for data storage comprising:
a computing system comprising at least one processor and instructions executable by the at least one processor to perform one or more operations comprising:
receiving digital information;
encoding digital information in one or more nucleic acid sequences; and
synthesizing a library of polynucleotides corresponding to the nucleic acid sequences; and
a modular rack-mount synthesis unit, wherein the modular rack-mount synthesis unit comprises:
a computer controller;
one or more reservoirs;
at least one flow cell block comprising one or more flow cells;
an extraction stage;
a post-processing unit;
a storage unit;
a robotic interface; and/or
a rack interface.

2. The system of claim 1, wherein the at least one flow cell block comprises at least 12 flow cells.

3. The system of claim 1, wherein the one or more flow cells comprise: one or more solid supports configured for synthesizing the library of polynucleotides; and one or more ports for exchanging gases, synthesis reagents, and/or extracted polynucleotides.

4. The system of claim 1, wherein the modular rack-mount synthesis unit comprises the extraction stage and/or the post-processing unit, and the extraction stage and/or the post-processing unit comprises:
an energy supply chain; and
one or more ports for exchanging synthesis reagents and/or extracted polynucleotides.

5. The system of claim 1, wherein the modular rack-mount synthesis unit comprises the storage unit, and the storage unit comprises:
a storage plate;
a robotic interface configured to move storage plates; and
an access port.

6. The system of claim 1, wherein the modular rack-mount synthesis unit comprises the rack interface, and the rack interface comprises:
a power supply;
a rack reagent bulkhead;
a synthesis unit reagent bulkhead;
one or more reservoirs;
an energy supply chain; and
a pilot valve bank.

7. The system of claim 1, wherein the storage unit provides the library of polynucleotides to a sequencing unit configured to sequence the library of polynucleotides.

8. The system of claim 1, further comprising one or more sensors configured to:
detect a status, wherein the status comprises a temperature, a pressure, a humidity, a change in voltage, a change in current, a capacitance, a conductivity, a storage plate position, a liquid volume, a flow rate, and/or a presence of a liquid; and
report the status to the computing system.

9. The system of claim 8, wherein the modular rack-mount synthesis unit comprises the at least one flow cell block, and the one or more sensors are further configured to pause synthesis at the one or more flow cells or the at least one flow cell block.

10. The system of claim 9, wherein the system is a first system for data storage, and wherein the computing system is configured to divert encoded data from (i) a paused flow cell or (ii) a paused flow cell block to an active flow cell, an active flow cell block, or to a second system for data storage.

11. The system of claim 1, wherein the computing system further comprises a cache, wherein digital information is retained in the cache until polynucleotides encoding the digital information are stored.

12. An assembly for storing information comprising:
a plurality of compartments comprising a first material, wherein the plurality of compartments is configured to receive a plurality of polynucleotides encoding information;
a base plate comprising a second material; and
a cover plate comprising a third material,
wherein a coefficient of thermal expansion (CTE) of the first material, the second material, or both, is less than the third material.

13. The assembly of claim 12, wherein the plurality of compartments are positioned in an array on the base plate.

14. The assembly of claim 13, wherein the base plate, the cover plate, or both, comprise a plurality of recessed features, and wherein each compartment is located at least partially within a recessed feature.

15. The assembly of claim 12, wherein the first material comprises borosilicate.

16. The assembly of claim 12, wherein the second material has a specific heat capacity of about 0.5 J/k-° C. to 2.5 J/k-° C.

17. The assembly of claim 12, wherein the third material includes:
a CTE of about 15 μm/m-° C. to 20 μm/m-° C.;
a thermal conductivity of about 15 W/m-K to about 20 W/m-K; and/or
a specific heat capacity of about 0.5 J/g-° C.

18. The assembly of claim 12, wherein the third material comprises stainless steel.

19. A system, comprising:
a rack defining a plurality of slots;
a synthesis unit configured to be removably inserted in at least one of the plurality of slots, the synthesis unit configured to synthesize a plurality of polynucleotides based on encoded digital information;
a storage unit configured to be removably inserted in at least one of the plurality of slots, the storage unit configured to be in fluidic communication with the synthesis unit to receive the synthesized plurality of nucleotides from the synthesis unit, dry, and store the synthesized plurality of polynucleotides; and
a sequencing unit configured to selectively receive at least a portion of the stored plurality of polynucleotides and sequence at least the portion of the stored plurality of polynucleotides, the sequence corresponding to a segment of the encoded digital information encoded at least the portion of the stored plurality of polynucleotides.

20. The system of claim 19, wherein the synthesis unit comprises:
a plurality of flow cells configured to allow reagents to at least one of incubate or flow therethrough, and to enable synthesis of the plurality of polynucleotides thereon, each of the plurality of flow cells including a solid support configured for polynucleotide synthesis, and one or more ports for fluid exchange.

21. The system of claim 20, wherein each of the plurality of flow cells is configured to:
receive a deprotection solution to deprotect a polynucleotide disposed thereon to allow addition of an additional nucleotide thereto;
receive a capping solution to cap the polynucleotide to inhibit attachment of an additional nucleotide to the polynucleotide; and
cleave a synthesized polynucleotide of the plurality of polynucleotides to decouple the synthesized polynucleotide from the solid support.

22. The system of claim 19, wherein the storage unit comprises:
at least one storage tray configured to receive the synthesized plurality of polynucleotides, store, the plurality of polynucleotides, and communicate the stored plurality of polynucleotides to the sequencing unit.

23. The system of claim 22, further comprising:
a robotic interface configured to at least one of insert or remove at least one of the synthesis unit, the storage unit from a corresponding slot in the rack.

24. The system of claim 23, wherein the robotic interface is configured or move the at least one storage tray between the synthesis unit, the storage unit, and the sequencing unit.

25. The system of claim 22, further comprising:
a tag coupled to the at least one storage tray, the tag configured to store metadata corresponding to plurality of polynucleotides stored in the at least one storage tray.

26. The system of claim 22, wherein the at least one storage tray comprises:
a plurality of compartments comprising a first material, wherein the plurality of compartments is configured to receive a plurality of polynucleotides encoding information;
a base plate comprising a second material; and
a cover plate comprising a third material,
wherein a coefficient of thermal expansion (CTE) of the first material, the second material, or both, is less than the third material.

27. The system of claim 19, further comprising:
a rack interface configured to interface with at least one of the synthesis unit, the storage unit, or the sequencing unit, or the sequencing unit, the rack interface including a power supply, one or more reservoirs, an energy supply chain, and a valve bank.

28. The system of claim 19, further comprising:
a controller configured to:
receive digital information;
encode the digital information in one or more nucleic acid sequences; and
communicate instructions to the synthesis unit to synthesize the plurality of polynucleotides based on the encoded digital information.

29. The system of claim 28, wherein the controller is further configured to:
detect a portion of the synthesis unit if paused; and
divert encoded digital information to an active portion of the synthesis unit.
